SEQUENCE LISTING (495 sequences)

SEQ ID NO: 1 - DNA, 156 bp, synthetic construct
  source 1..156: mol_type = other DNA
gacctcggctcacagcgcgccgttttagagctagaaatagcaagttaaaataaggctagtccgttatcaacttgaaaaagtggcaccgagtcggtgcccggatgatcctgacgacggagaccgccgtcgtcgacaagccggccgcgctgtgagccg

SEQ ID NO: 2 - DNA, 155 bp, synthetic construct
  source 1..155: mol_type = other DNA
gaagccggccttgcacatgcgttttagagctagaaatagcaagttaaaataaggctagtccgttatcaacttgaaaaagtggcaccgagtcggtgcggccggcttgtcgacgacggcggtctccgtcgtcaggatcatccggtgtgcaaggccgg

SEQ ID NO: 3 - DNA, 156 bp, synthetic construct; identical to SEQ ID NO: 1
  source 1..156: mol_type = other DNA

SEQ ID NO: 4 - DNA, 155 bp, synthetic construct
  source 1..155: mol_type = other DNA
ggcatcgtcgcccgcgaagcgttttagagctagaaatagcaagttaaaataaggctagtccgttatcaacttgaaaaagtggcaccgagtcggtgcggccggcttgtcgacgacggcggtctccgtcgtcaggatcatccggtcgcgggcgacga

SEQ ID NO: 5 - DNA, 156 bp, synthetic construct; identical to SEQ ID NO: 1
  source 1..156: mol_type = other DNA

SEQ ID NO: 6 - DNA, 155 bp, synthetic construct
  source 1..155: mol_type = other DNA
ggaggggaagacggcccggggttttagagctagaaatagcaagttaaaataaggctagtccgttatcaacttgaaaaagtggcaccgagtcggtgcggccggcttgtcgacgacggcggtctccgtcgtcaggatcatccgggggccgtcttccc

SEQ ID NO: 7 - DNA, 156 bp, synthetic construct; identical to SEQ ID NO: 1
  source 1..156: mol_type = other DNA

SEQ ID NO: 8 - DNA, 156 bp, synthetic construct
  source 1..156: mol_type = other DNA
gtcttcccctccatcgtgggggttttagagctagaaatagcaagttaaaataaggctagtccgttatcaacttgaaaaagtggcaccgagtcggtgcggccggcttgtcgacgacggcggtctccgtcgtcaggatcatccggcacgatggagggg

SEQ ID NO: 9 - DNA, 156 bp, synthetic construct; identical to SEQ ID NO: 1
  source 1..156: mol_type = other DNA

SEQ ID NO: 10 - DNA, 156 bp, synthetic construct
  source 1..156: mol_type = other DNA
gctggggcgccccacgatggagttttagagctagaaatagcaagttaaaataaggctagtccgttatcaacttgaaaaagtggcaccgagtcggtgcggccggcttgtcgacgacggcggtctccgtcgtcaggatcatccggatcgtggggcgcc

SEQ ID NO: 11 - DNA, 155 bp, synthetic construct
  source 1..155: mol_type = other DNA
gctattctcgcagctcaccagttttagagctagaaatagcaagttaaaataaggctagtccgttatcaacttgaaaaagtggcaccgagtcggtgcccggatgatcctgacgacggagaccgccgtcgtcgacaagccggcctgagctgcgagaa

SEQ ID NO: 12 - DNA, 155 bp, synthetic construct; identical to SEQ ID NO: 2
  source 1..155: mol_type = other DNA

SEQ ID NO: 13 - DNA, 155 bp, synthetic construct; identical to SEQ ID NO: 11
  source 1..155: mol_type = other DNA

SEQ ID NO: 14 - DNA, 155 bp, synthetic construct; identical to SEQ ID NO: 4
  source 1..155: mol_type = other DNA

SEQ ID NO: 15 - DNA, 155 bp, synthetic construct; identical to SEQ ID NO: 11
  source 1..155: mol_type = other DNA

SEQ ID NO: 16 - DNA, 155 bp, synthetic construct; identical to SEQ ID NO: 6
  source 1..155: mol_type = other DNA

SEQ ID NO: 17 - DNA, 155 bp, synthetic construct; identical to SEQ ID NO: 11
  source 1..155: mol_type = other DNA

SEQ ID NO: 18 - DNA, 156 bp, synthetic construct; identical to SEQ ID NO: 8
  source 1..156: mol_type = other DNA

SEQ ID NO: 19 - DNA, 155 bp, synthetic construct; identical to SEQ ID NO: 11
  source 1..155: mol_type = other DNA

SEQ ID NO: 20 - DNA, 156 bp, synthetic construct; identical to SEQ ID NO: 10
  source 1..156: mol_type = other DNA

SEQ ID NO: 21 - DNA, 155 bp, synthetic construct
  source 1..155: mol_type = other DNA
gccgcgctcgtcgtcgacaagttttagagctagaaatagcaagttaaaataaggctagtccgttatcaacttgaaaaagtggcaccgagtcggtgcccggatgatcctgacgacggagaccgccgtcgtcgacaagccggcctcgacgacgagcg

SEQ ID NO: 22 - DNA, 155 bp, synthetic construct; identical to SEQ ID NO: 2
  source 1..155: mol_type = other DNA

SEQ ID NO: 23 - DNA, 155 bp, synthetic construct; identical to SEQ ID NO: 21
  source 1..155: mol_type = other DNA

SEQ ID NO: 24 - DNA, 155 bp, synthetic construct; identical to SEQ ID NO: 4
  source 1..155: mol_type = other DNA

SEQ ID NO: 25 - DNA, 155 bp, synthetic construct; identical to SEQ ID NO: 21
  source 1..155: mol_type = other DNA

SEQ ID NO: 26 - DNA, 155 bp, synthetic construct; identical to SEQ ID NO: 6
  source 1..155: mol_type = other DNA

SEQ ID NO: 27 - DNA, 155 bp, synthetic construct; identical to SEQ ID NO: 21
  source 1..155: mol_type = other DNA

SEQ ID NO: 28 - DNA, 156 bp, synthetic construct; identical to SEQ ID NO: 8
  source 1..156: mol_type = other DNA

SEQ ID NO: 29 - DNA, 155 bp, synthetic construct; identical to SEQ ID NO: 21
  source 1..155: mol_type = other DNA

SEQ ID NO: 30 - DNA, 156 bp, synthetic construct; identical to SEQ ID NO: 10
  source 1..156: mol_type = other DNA

SEQ ID NO: 31 - DNA, 156 bp, synthetic construct
  source 1..156: mol_type = other DNA
gctcgtcgtcgacaacggctcgttttagagctagaaatagcaagttaaaataaggctagtccgttatcaacttgaaaaagtggcaccgagtcggtgcccggatgatcctgacgacggagaccgccgtcgtcgacaagccggccccgttgtcgacga

SEQ ID NO: 32 - DNA, 155 bp, synthetic construct; identical to SEQ ID NO: 2
  source 1..155: mol_type = other DNA

SEQ ID NO: 33 - DNA, 156 bp, synthetic construct; identical to SEQ ID NO: 31
  source 1..156: mol_type = other DNA

SEQ ID NO: 34 - DNA, 155 bp, synthetic construct; identical to SEQ ID NO: 4
  source 1..155: mol_type = other DNA

SEQ ID NO: 35 - DNA, 156 bp, synthetic construct; identical to SEQ ID NO: 31
  source 1..156: mol_type = other DNA

SEQ ID NO: 36 - DNA, 155 bp, synthetic construct; identical to SEQ ID NO: 6
  source 1..155: mol_type = other DNA

SEQ ID NO: 37 - DNA, 156 bp, synthetic construct; identical to SEQ ID NO: 31
  source 1..156: mol_type = other DNA

SEQ ID NO: 38 - DNA, 156 bp, synthetic construct; identical to SEQ ID NO: 8
  source 1..156: mol_type = other DNA

SEQ ID NO: 39 - DNA, 156 bp, synthetic construct; identical to SEQ ID NO: 31
  source 1..156: mol_type = other DNA

SEQ ID NO: 40 - DNA, 156 bp, synthetic construct; identical to SEQ ID NO: 10
  source 1..156: mol_type = other DNA

SEQ ID NO: 41 - DNA, 140 bp, synthetic construct
  source 1..140: mol_type = other DNA
gacctcggctcacagcgcgccgttttagagctagaaatagcaagttaaaataaggctagtccgttatcaacttgaaaaagtggcaccgagtcggtgcacggagaccgccgtcgtcgacaagccggccgcgctgtgagccg

SEQ ID NO: 42 - DNA, 139 bp, synthetic construct
  source 1..139: mol_type = other DNA
ggcatcgtcgcccgcgaagcgttttagagctagaaatagcaagttaaaataaggctagtccgttatcaacttgaaaaagtggcaccgagtcggtgcacggcggtctccgtcgtcaggatcatccggtcgcgggcgacga

SEQ ID NO: 43 - DNA, 140 bp, synthetic construct; identical to SEQ ID NO: 41
  source 1..140: mol_type = other DNA

SEQ ID NO: 44 - DNA, 139 bp, synthetic construct
  source 1..139: mol_type = other DNA
ggaggggaagacggcccggggttttagagctagaaatagcaagttaaaataaggctagtccgttatcaacttgaaaaagtggcaccgagtcggtgcacggcggtctccgtcgtcaggatcatccgggggccgtcttccc

SEQ ID NO: 45 - DNA, 140 bp, synthetic construct; identical to SEQ ID NO: 41
  source 1..140: mol_type = other DNA

SEQ ID NO: 46 - DNA, 140 bp, synthetic construct
  source 1..140: mol_type = other DNA
gtcttcccctccatcgtgggggttttagagctagaaatagcaagttaaaataaggctagtccgttatcaacttgaaaaagtggcaccgagtcggtgcacggcggtctccgtcgtcaggatcatccggcacgatggagggg

SEQ ID NO: 47 - DNA, 140 bp, synthetic construct; identical to SEQ ID NO: 41
  source 1..140: mol_type = other DNA

SEQ ID NO: 48 - DNA, 140 bp, synthetic construct
  source 1..140: mol_type = other DNA
gctggggcgccccacgatggagttttagagctagaaatagcaagttaaaataaggctagtccgttatcaacttgaaaaagtggcaccgagtcggtgcacggcggtctccgtcgtcaggatcatccggatcgtggggcgcc

SEQ ID NO: 49 - DNA, 139 bp, synthetic construct
  source 1..139: mol_type = other DNA
gctattctcgcagctcaccagttttagagctagaaatagcaagttaaaataaggctagtccgttatcaacttgaaaaagtggcaccgagtcggtgcacggagaccgccgtcgtcgacaagccggcctgagctgcgagaa

SEQ ID NO: 50 - DNA, 156 bp, synthetic construct
  source 1..156: mol_type = other DNA
gcggtagtgacgcgtattgccgttttagagctagaaatagcaagttaaaataaggctagtccgttatcaacttgaaaaagtggcaccgagtcggtgcccggatgatcctgacgacggagaccgccgtcgtcgacaagccggccaatacgcgtcact

SEQ ID NO: 51 - DNA, 139 bp, synthetic construct; identical to SEQ ID NO: 49
  source 1..139: mol_type = other DNA

SEQ ID NO: 52 - DNA, 139 bp, synthetic construct; identical to SEQ ID NO: 42
  source 1..139: mol_type = other DNA

SEQ ID NO: 53 - DNA, 139 bp, synthetic construct; identical to SEQ ID NO: 49
  source 1..139: mol_type = other DNA

SEQ ID NO: 54 - DNA, 139 bp, synthetic construct; identical to SEQ ID NO: 44
  source 1..139: mol_type = other DNA

SEQ ID NO: 55 - DNA, 139 bp, synthetic construct; identical to SEQ ID NO: 49
  source 1..139: mol_type = other DNA

SEQ ID NO: 56 - DNA, 140 bp, synthetic construct; identical to SEQ ID NO: 46
  source 1..140: mol_type = other DNA

SEQ ID NO: 57 - DNA, 139 bp, synthetic construct
  source 1..139: mol_type = other DNA
gccgcgctcgtcgtcgacaagttttagagctagaaatagcaagttaaaataaggctagtccgttatcaacttgaaaaagtggcaccgagtcggtgcacggagaccgccgtcgtcgacaagccggcctcgacgacgagcg

SEQ ID NO: 58 - DNA, 140 bp, synthetic construct; identical to SEQ ID NO: 48
  source 1..140: mol_type = other DNA

SEQ ID NO: 59 - DNA, 139 bp, synthetic construct; identical to SEQ ID NO: 57
  source 1..139: mol_type = other DNA

SEQ ID NO: 60 - DNA, 156 bp, synthetic construct; identical to SEQ ID NO: 50
  source 1..156: mol_type = other DNA

SEQ ID NO: 61 - DNA, 139 bp, synthetic construct; identical to SEQ ID NO: 57
  source 1..139: mol_type = other DNA

SEQ ID NO: 62 - DNA, 139 bp, synthetic construct; identical to SEQ ID NO: 42
  source 1..139: mol_type = other DNA

SEQ ID NO: 63 - DNA, 139 bp, synthetic construct; identical to SEQ ID NO: 57
  source 1..139: mol_type = other DNA

SEQ ID NO: 64 - DNA, 139 bp, synthetic construct; identical to SEQ ID NO: 44
  source 1..139: mol_type = other DNA

SEQ ID NO: 65 - DNA, 140 bp, synthetic construct
  source 1..140: mol_type = other DNA
gctcgtcgtcgacaacggctcgttttagagctagaaatagcaagttaaaataaggctagtccgttatcaacttgaaaaagtggcaccgagtcggtgcacggagaccgccgtcgtcgacaagccggccccgttgtcgacga

SEQ ID NO: 66 - DNA, 140 bp, synthetic construct; identical to SEQ ID NO: 46
  source 1..140: mol_type = other DNA

SEQ ID NO: 67 - DNA, 140 bp, synthetic construct; identical to SEQ ID NO: 65
  source 1..140: mol_type = other DNA

SEQ ID NO: 68 - DNA, 140 bp, synthetic construct; identical to SEQ ID NO: 48
  source 1..140: mol_type = other DNA

SEQ ID NO: 69 - DNA, 140 bp, synthetic construct; identical to SEQ ID NO: 65
  source 1..140: mol_type = other DNA

SEQ ID NO: 70 - DNA, 156 bp, synthetic construct; identical to SEQ ID NO: 50
  source 1..156: mol_type = other DNA

SEQ ID NO: 71 - DNA, 140 bp, synthetic construct; identical to SEQ ID NO: 65
  source 1..140: mol_type = other DNA

SEQ ID NO: 72 - DNA, 139 bp, synthetic construct; identical to SEQ ID NO: 42
  source 1..139: mol_type = other DNA

SEQ ID NO: 73 - DNA, 139 bp, synthetic construct
  source 1..139: mol_type = other DNA
gaagccggccttgcacatgcgttttagagctagaaatagcaagttaaaataaggctagtccgttatcaacttgaaaaagtggcaccgagtcggtgcacggcggtctccgtcgtcaggatcatccggtgtgcaaggccgg

SEQ ID NO: 74 - DNA, 139 bp, synthetic construct; identical to SEQ ID NO: 44
  source 1..139: mol_type = other DNA

SEQ ID NO: 75 - DNA, 139 bp, synthetic construct; identical to SEQ ID NO: 73
  source 1..139: mol_type = other DNA

SEQ ID NO: 76 - DNA, 140 bp, synthetic construct; identical to SEQ ID NO: 46
  source 1..140: mol_type = other DNA

SEQ ID NO: 77 - DNA, 139 bp, synthetic construct; identical to SEQ ID NO: 73
  source 1..139: mol_type = other DNA

SEQ ID NO: 78 - DNA, 140 bp, synthetic construct; identical to SEQ ID NO: 48
  source 1..140: mol_type = other DNA

SEQ ID NO: 79 - DNA, 139 bp, synthetic construct; identical to SEQ ID NO: 73
  source 1..139: mol_type = other DNA

SEQ ID NO: 80 - DNA, 156 bp, synthetic construct; identical to SEQ ID NO: 50
  source 1..156: mol_type = other DNA

SEQ ID NO: 81 - DNA, 156 bp, synthetic construct
  source 1..156: mol_type = other DNA
gcgggctggagctgttcgcgcgttttagagctagaaatagcaagttaaaataaggctagtccgttatcaacttgaaaaagtggcaccgagtcggtgcggccggcttgtcgacgacggcggtctccgtcgtcaggatcatccggcgaacagctccag

SEQ ID NO: 82 - DNA, 156 bp, synthetic construct
  source 1..156: mol_type = other DNA
gccgcgcgcgcgaaaaagccggttttagagctagaaatagcaagttaaaataaggctagtccgttatcaacttgaaaaagtggcaccgagtcggtgcccggatgatcctgacgacggagaccgccgtcgtcgacaagccggccctttttcgcgcgc

SEQ ID NO: 83 - DNA, 156 bp, synthetic construct; identical to SEQ ID NO: 81
  source 1..156: mol_type = other DNA

SEQ ID NO: 84 - DNA, 156 bp, synthetic construct
  source 1..156: mol_type = other DNA
gttccgcgcgcgcgaaaaagcgttttagagctagaaatagcaagttaaaataaggctagtccgttatcaacttgaaaaagtggcaccgagtcggtgcccggatgatcctgacgacggagaccgccgtcgtcgacaagccggccttttcgcgcgcgc

SEQ ID NO: 85 - DNA, 156 bp, synthetic construct; identical to SEQ ID NO: 81
  source 1..156: mol_type = other DNA

SEQ ID NO: 86 - DNA, 156 bp, synthetic construct
  source 1..156: mol_type = other DNA
gttgcgccgccccctcccaatgttttagagctagaaatagcaagttaaaataaggctagtccgttatcaacttgaaaaagtggcaccgagtcggtgcccggatgatcctgacgacggagaccgccgtcgtcgacaagccggccgggagggggcggc

SEQ ID NO: 87 - DNA, 156 bp, synthetic construct; identical to SEQ ID NO: 81
  source 1..156: mol_type = other DNA

SEQ ID NO: 88 - DNA, 156 bp, synthetic construct
  source 1..156: mol_type = other DNA
gccccactctcttgccctgtggttttagagctagaaatagcaagttaaaataaggctagtccgttatcaacttgaaaaagtggcaccgagtcggtgcccggatgatcctgacgacggagaccgccgtcgtcgacaagccggccagggcaagagagt

SEQ ID NO: 89 - DNA, 155 bp, synthetic construct
  source 1..155: mol_type = other DNA
gggaggcaagcgcaggcactgttttagagctagaaatagcaagttaaaataaggctagtccgttatcaacttgaaaaagtggcaccgagtcggtgcggccggcttgtcgacgacggcggtctccgtcgtcaggatcatccgggcctgcgcttgcc

SEQ ID NO: 90 - DNA, 156 bp, synthetic construct; identical to SEQ ID NO: 82
  source 1..156: mol_type = other DNA

SEQ ID NO: 91 - DNA, 155 bp, synthetic construct; identical to SEQ ID NO: 89
  source 1..155: mol_type = other DNA

SEQ ID NO: 92 - DNA, 156 bp, synthetic construct; identical to SEQ ID NO: 84
  source 1..156: mol_type = other DNA

SEQ ID NO: 93 - DNA, 155 bp, synthetic construct; identical to SEQ ID NO: 89
  source 1..155: mol_type = other DNA

SEQ ID NO: 94 - DNA, 156 bp, synthetic construct; identical to SEQ ID NO: 86
  source 1..156: mol_type = other DNA

SEQ ID NO: 95 - DNA, 155 bp, synthetic construct; identical to SEQ ID NO: 89
  source 1..155: mol_type = other DNA

SEQ ID NO: 96 - DNA, 156 bp, synthetic construct; identical to SEQ ID NO: 88
  source 1..156: mol_type = other DNA

SEQ ID NO: 97 - DNA, 155 bp, synthetic construct
  source 1..155: mol_type = other DNA
gtccgggagcgagcctgccggttttagagctagaaatagcaagttaaaataaggctagtccgttatcaacttgaaaaagtggcaccgagtcggtgcggccggcttgtcgacgacggcggtctccgtcgtcaggatcatccggcaggctcgctccc

SEQ ID NO: 98 - DNA, 156 bp, synthetic construct; identical to SEQ ID NO: 82
  source 1..156: mol_type = other DNA

SEQ ID NO: 99 - DNA, 155 bp, synthetic construct; identical to SEQ ID NO: 97
  source 1..155: mol_type = other DNA

SEQ ID NO: 100 - DNA, 156 bp, synthetic construct; identical to SEQ ID NO: 84
  source 1..156: mol_type = other DNA

SEQ ID NO: 101 - DNA, 155 bp, synthetic construct; identical to SEQ ID NO: 97
  source 1..155: mol_type = other DNA

SEQ ID NO: 102 - DNA, 156 bp, synthetic construct; identical to SEQ ID NO: 86
  source 1..156: mol_type = other DNA

SEQ ID NO: 103 - DNA, 155 bp, synthetic construct; identical to SEQ ID NO: 97
  source 1..155: mol_type = other DNA

SEQ ID NO: 104 - DNA, 156 bp, synthetic construct; identical to SEQ ID NO: 88
  source 1..156: mol_type = other DNA

SEQ ID NO: 105 - DNA, 156 bp, synthetic construct
  source 1..156: mol_type = other DNA
gtgttcgcgctggcatcttgcgttttagagctagaaatagcaagttaaaataaggctagtccgttatcaacttgaaaaagtggcaccgagtcggtgcggccggcttgtcgacgacggcggtctccgtcgtcaggatcatccggagatgccagcgcg

SEQ ID NO: 106 - DNA, 156 bp, synthetic construct; identical to SEQ ID NO: 82
  source 1..156: mol_type = other DNA

SEQ ID NO: 107 - DNA, 156 bp, synthetic construct; identical to SEQ ID NO: 105
  source 1..156: mol_type = other DNA

SEQ ID NO: 108 - DNA, 156 bp, synthetic construct; identical to SEQ ID NO: 84
  source 1..156: mol_type = other DNA

SEQ ID NO: 109 - DNA, 156 bp, synthetic construct; identical to SEQ ID NO: 105
  source 1..156: mol_type = other DNA

SEQ ID NO: 110 - DNA, 156 bp, synthetic construct; identical to SEQ ID NO: 86
  source 1..156: mol_type = other DNA

SEQ ID NO: 111 - DNA, 156 bp, synthetic construct; identical to SEQ ID NO: 105
  source 1..156: mol_type = other DNA

SEQ ID NO: 112 - DNA, 156 bp, synthetic construct; identical to SEQ ID NO: 88
  source 1..156: mol_type = other DNA

SEQ ID NO: 113 - DNA, 156 bp, synthetic construct
  source 1..156: mol_type = other DNA
gaacagctctgaacgagacccgttttagagctagaaatagcaagttaaaataaggctagtccgttatcaacttgaaaaagtggcaccgagtcggtgcggccggcttgtcgacgacggcggtctccgtcgtcaggatcatccggtctcgttcagagc

SEQ ID NO: 114 - DNA, 156 bp, synthetic construct; identical to SEQ ID NO: 82
  source 1..156: mol_type = other DNA

SEQ ID NO: 115 - DNA, 156 bp, synthetic construct; identical to SEQ ID NO: 113
  source 1..156: mol_type = other DNA

SEQ ID NO: 116 - DNA, 156 bp, synthetic construct; identical to SEQ ID NO: 84
  source 1..156: mol_type = other DNA

SEQ ID NO: 117 - DNA, 156 bp, synthetic construct; identical to SEQ ID NO: 113
  source 1..156: mol_type = other DNA

SEQ ID NO: 118 - DNA, 156 bp, synthetic construct; identical to SEQ ID NO: 86
  source 1..156: mol_type = other DNA

SEQ ID NO: 119 - DNA, 156 bp, synthetic construct; identical to SEQ ID NO: 113
  source 1..156: mol_type = other DNA

SEQ ID NO: 120 - DNA, 156 bp, synthetic construct; identical to SEQ ID NO: 88
  source 1..156: mol_type = other DNA

SEQ ID NO: 121 - DNA, 140 bp, synthetic construct
  source 1..140: mol_type = other DNA
gcgggctggagctgttcgcgcgttttagagctagaaatagcaagttaaaataaggctagtccgttatcaacttgaaaaagtggcaccgagtcggtgcacggcggtctccgtcgtcaggatcatccggcgaacagctccag

SEQ ID NO: 122 - DNA, 140 bp, synthetic construct
  source 1..140: mol_type = other DNA
gccgcgcgcgcgaaaaagccggttttagagctagaaatagcaagttaaaataaggctagtccgttatcaacttgaaaaagtggcaccgagtcggtgcacggagaccgccgtcgtcgacaagccggccctttttcgcgcgc

SEQ ID NO: 123 - DNA, 140 bp, synthetic construct; identical to SEQ ID NO: 121
  source 1..140: mol_type = other DNA

SEQ ID NO: 124 - DNA, 140 bp, synthetic construct
  source 1..140: mol_type = other DNA
gttccgcgcgcgcgaaaaagcgttttagagctagaaatagcaagttaaaataaggctagtccgttatcaacttgaaaaagtggcaccgagtcggtgcacggagaccgccgtcgtcgacaagccggccttttcgcgcgcgc

SEQ ID NO: 125 - DNA, 140 bp, synthetic construct; identical to SEQ ID NO: 121
  source 1..140: mol_type = other DNA

SEQ ID NO: 126 - DNA, 140 bp, synthetic construct
  source 1..140: mol_type = other DNA
gttgcgccgccccctcccaatgttttagagctagaaatagcaagttaaaataaggctagtccgttatcaacttgaaaaagtggcaccgagtcggtgcacggagaccgccgtcgtcgacaagccggccgggagggggcggc

SEQ ID NO: 127 - DNA, 140 bp, synthetic construct; identical to SEQ ID NO: 121
  source 1..140: mol_type = other DNA

SEQ ID NO: 128 - DNA, 140 bp, synthetic construct
  source 1..140: mol_type = other DNA
gccccactctcttgccctgtggttttagagctagaaatagcaagttaaaataaggctagtccgttatcaacttgaaaaagtggcaccgagtcggtgcacggagaccgccgtcgtcgacaagccggccagggcaagagagt

SEQ ID NO: 129 - DNA, 139 bp, synthetic construct
  source 1..139: mol_type = other DNA
gggaggcaagcgcaggcactgttttagagctagaaatagcaagttaaaataaggctagtccgttatcaacttgaaaaagtggcaccgagtcggtgcacggcggtctccgtcgtcaggatcatccgggcctgcgcttgcc

SEQ ID NO: 130 - DNA, 140 bp, synthetic construct; identical to SEQ ID NO: 122
  source 1..140: mol_type = other DNA

SEQ ID NO: 131 - DNA, 139 bp, synthetic construct; identical to SEQ ID NO: 129
  source 1..139: mol_type = other DNA

SEQ ID NO: 132 - DNA, 140 bp, synthetic construct; identical to SEQ ID NO: 124
  source 1..140: mol_type = other DNA

SEQ ID NO: 133 - DNA, 139 bp, synthetic construct; identical to SEQ ID NO: 129
  source 1..139: mol_type = other DNA

SEQ ID NO: 134 - DNA, 140 bp, synthetic construct; identical to SEQ ID NO: 126
  source 1..140: mol_type = other DNA

SEQ ID NO: 135 - DNA, 139 bp, synthetic construct; identical to SEQ ID NO: 129
  source 1..139: mol_type = other DNA

SEQ ID NO: 136 - DNA, 140 bp, synthetic construct; identical to SEQ ID NO: 128
  source 1..140: mol_type = other DNA

SEQ ID NO: 137 - DNA, 139 bp, synthetic construct
  source 1..139: mol_type = other DNA
gtccgggagcgagcctgccggttttagagctagaaatagcaagttaaaataaggctagtccgttatcaacttgaaaaagtggcaccgagtcggtgcacggcggtctccgtcgtcaggatcatccggcaggctcgctccc

SEQ ID NO: 138 - DNA, 140 bp, synthetic construct; identical to SEQ ID NO: 122
  source 1..140: mol_type = other DNA

SEQ ID NO: 139 - DNA, 139 bp, synthetic construct; identical to SEQ ID NO: 137
  source 1..139: mol_type = other DNA

SEQ ID NO: 140 - DNA, 140 bp, synthetic construct; identical to SEQ ID NO: 124
  source 1..140: mol_type = other DNA

SEQ ID NO: 141 - DNA, 139 bp, synthetic construct; identical to SEQ ID NO: 137
  source 1..139: mol_type = other DNA

SEQ ID NO: 142 - DNA, 140 bp, synthetic construct; identical to SEQ ID NO: 126
  source 1..140: mol_type = other DNA

SEQ ID NO: 143 - DNA, 139 bp, synthetic construct; identical to SEQ ID NO: 137
  source 1..139: mol_type = other DNA

SEQ ID NO: 144 - DNA, 140 bp, synthetic construct; identical to SEQ ID NO: 128
  source 1..140: mol_type = other DNA

SEQ ID NO: 145 - DNA, 140 bp, synthetic construct
  source 1..140: mol_type = other DNA
gtgttcgcgctggcatcttgcgttttagagctagaaatagcaagttaaaataaggctagtccgttatcaacttgaaaaagtggcaccgagtcggtgcacggcggtctccgtcgtcaggatcatccggagatgccagcgcg

SEQ ID NO: 146 - DNA, 140 bp, synthetic construct; identical to SEQ ID NO: 122
  source 1..140: mol_type = other DNA

SEQ ID NO: 147 - DNA, 140 bp, synthetic construct; identical to SEQ ID NO: 145
  source 1..140: mol_type = other DNA

SEQ ID NO: 148 - DNA, 140 bp, synthetic construct; identical to SEQ ID NO: 124
  source 1..140: mol_type = other DNA

SEQ ID NO: 149 - DNA, 140 bp, synthetic construct; identical to SEQ ID NO: 145
  source 1..140: mol_type = other DNA

SEQ ID NO: 150 - DNA, 140 bp, synthetic construct; identical to SEQ ID NO: 126
  source 1..140: mol_type = other DNA

SEQ ID NO: 151 - DNA, 140 bp, synthetic construct; identical to SEQ ID NO: 145
  source 1..140: mol_type = other DNA

SEQ ID NO: 152 - DNA, 140 bp, synthetic construct; identical to SEQ ID NO: 128
  source 1..140: mol_type = other DNA

SEQ ID NO: 153 - DNA, 140 bp, synthetic construct
  source 1..140: mol_type = other DNA
gaacagctctgaacgagacccgttttagagctagaaatagcaagttaaaataaggctagtccgttatcaacttgaaaaagtggcaccgagtcggtgcacggcggtctccgtcgtcaggatcatccggtctcgttcagagc

SEQ ID NO: 154 - DNA, 140 bp, synthetic construct; identical to SEQ ID NO: 122
  source 1..140: mol_type = other DNA

SEQ ID NO: 155 - DNA, 140 bp, synthetic construct; identical to SEQ ID NO: 153
  source 1..140: mol_type = other DNA

SEQ ID NO: 156 - DNA, 140 bp, synthetic construct; identical to SEQ ID NO: 124
  source 1..140: mol_type = other DNA

SEQ ID NO: 157 - DNA, 140 bp, synthetic construct; identical to SEQ ID NO: 153
  source 1..140: mol_type = other DNA

SEQ ID NO: 158 - DNA, 140 bp, synthetic construct; identical to SEQ ID NO: 126
  source 1..140: mol_type = other DNA

SEQ ID NO: 159 - DNA, 140 bp, synthetic construct; identical to SEQ ID NO: 153
  source 1..140: mol_type = other DNA

SEQ ID NO: 160 - DNA, 140 bp, synthetic construct; identical to SEQ ID NO: 128
  source 1..140: mol_type = other DNA

SEQ ID NO: 161 - DNA, 150 bp, synthetic construct
  source 1..150: mol_type = other DNA
gcttgtcggctttagaagttagttttagagctagaaatagcaagttaaaataaggctagtccgttatcaacttgaaaaagtggcaccgagtcggtgcccctcgcctccttaaatgatcctgacgacggagaccgccgtcgtcgacaagcc

SEQ ID NO: 162 - DNA, 97 bp, synthetic construct
  source 1..97: mol_type = other DNA
gcagagaagctgggcagacaagttttagagctagaaatagcaagttaaaataaggctagtccgttatcaacttgaaaaagtggcaccgagtcggtgc

SEQ ID NO: 163 - DNA, 149 bp, synthetic construct
  source 1..149: mol_type = other DNA
gtcggctttagaagttaagggttttagagctagaaatagcaagttaaaataaggctagtccgttatcaacttgaaaaagtggcaccgagtcggtgccagccctcgcctcctatgatcctgacgacggagaccgccgtcgtcgacaagcc

SEQ ID NO: 164 - DNA, 97 bp, synthetic construct; identical to SEQ ID NO: 162
  source 1..97: mol_type = other DNA

SEQ ID NO: 165 - DNA, 150 bp, synthetic construct
  source 1..150: mol_type = other DNA
gctttagaagttaaggaggcggttttagagctagaaatagcaagttaaaataaggctagtccgttatcaacttgaaaaagtggcaccgagtcggtgcgaccccagccctcgcatgatcctgacgacggagaccgccgtcgtcgacaagcc

SEQ ID NO: 166 - DNA, 97 bp, synthetic construct; identical to SEQ ID NO: 162
  source 1..97: mol_type = other DNA

SEQ ID NO: 167 - DNA, 150 bp, synthetic construct
  source 1..150: mol_type = other DNA
gtttagaagttaaggaggcgagttttagagctagaaatagcaagttaaaataaggctagtccgttatcaacttgaaaaagtggcaccgagtcggtgcagaccccagccctcgatgatcctgacgacggagaccgccgtcgtcgacaagcc

SEQ ID NO: 168 - DNA, 97 bp, synthetic construct; identical to SEQ ID NO: 162
  source 1..97: mol_type = other DNA

SEQ ID NO: 169 - DNA, 149 bp, synthetic construct
  source 1..149: mol_type = other DNA
gaagttaaggaggcgagggcgttttagagctagaaatagcaagttaaaataaggctagtccgttatcaacttgaaaaagtggcaccgagtcggtgctgacagaccccagccatgatcctgacgacggagaccgccgtcgtcgacaagcc

SEQ ID NO: 170 - DNA, 97 bp, synthetic construct; identical to SEQ ID NO: 162
  source 1..97: mol_type = other DNA

SEQ ID NO: 171 - DNA, 150 bp, synthetic construct
  source 1..150: mol_type = other DNA
gaagttaaggaggcgagggctgttttagagctagaaatagcaagttaaaataaggctagtccgttatcaacttgaaaaagtggcaccgagtcggtgcctgacagaccccagcatgatcctgacgacggagaccgccgtcgtcgacaagcc

SEQ ID NO: 172 - DNA, 97 bp, synthetic construct; identical to SEQ ID NO: 162
  source 1..97: mol_type = other DNA

SEQ ID NO: 173 - DNA, 150 bp, synthetic construct
  source 1..150: mol_type = other DNA
gagttaaggaggcgagggctggttttagagctagaaatagcaagttaaaataaggctagtccgttatcaacttgaaaaagtggcaccgagtcggtgcactgacagaccccagatgatcctgacgacggagaccgccgtcgtcgacaagcc

SEQ ID NO: 174 - DNA, 97 bp, synthetic construct; identical to SEQ ID NO: 162
  source 1..97: mol_type = other DNA

SEQ ID NO: 175 - DNA, 150 bp, synthetic construct; identical to SEQ ID NO: 161
  source 1..150: mol_type = other DNA

SEQ ID NO: 176 - DNA, 96 bp, synthetic construct
  source 1..96: mol_type = other DNA
ggaaggtccgcagagaagctgttttagagctagaaatagcaagttaaaataaggctagtccgttatcaacttgaaaaagtggcaccgagtcggtgc

SEQ ID NO: 177 - DNA, 149 bp, synthetic construct; identical to SEQ ID NO: 163
  source 1..149: mol_type = other DNA

SEQ ID NO: 178 - DNA, 96 bp, synthetic construct; identical to SEQ ID NO: 176
  source 1..96: mol_type = other DNA

SEQ ID NO: 179 - DNA, 150 bp, synthetic construct; identical to SEQ ID NO: 165
  source 1..150: mol_type = other DNA

SEQ ID NO: 180 - DNA, 96 bp, synthetic construct; identical to SEQ ID NO: 176
  source 1..96: mol_type = other DNA

SEQ ID NO: 181 - DNA, 150 bp, synthetic construct; identical to SEQ ID NO: 167
  source 1..150: mol_type = other DNA

SEQ ID NO: 182 - DNA, 96 bp, synthetic construct; identical to SEQ ID NO: 176
  source 1..96: mol_type = other DNA

SEQ ID NO: 183 - DNA, 149 bp, synthetic construct; identical to SEQ ID NO: 169
  source 1..149: mol_type = other DNA

SEQ ID NO: 184 - DNA, 96 bp, synthetic construct; identical to SEQ ID NO: 176
  source 1..96: mol_type = other DNA

SEQ ID NO: 185 - DNA, 150 bp, synthetic construct; identical to SEQ ID NO: 171
  source 1..150: mol_type = other DNA

SEQ ID NO: 186 - DNA, 96 bp, synthetic construct; identical to SEQ ID NO: 176
  source 1..96: mol_type = other DNA

SEQ ID NO: 187 - DNA, 150 bp, synthetic construct; identical to SEQ ID NO: 173
  source 1..150: mol_type = other DNA

SEQ ID NO: 188 - DNA, 96 bp, synthetic construct; identical to SEQ ID NO: 176
  source 1..96: mol_type = other DNA

SEQ ID NO: 189 - DNA, 150 bp, synthetic construct; identical to SEQ ID NO: 161
  source 1..150: mol_type = other DNA

SEQ ID NO: 190 - DNA, 97 bp, synthetic construct
  source 1..97: mol_type = other DNA
gaggaaggtccgcagagaagcgttttagagctagaaatagcaagttaaaataaggctagtccgttatcaacttgaaaaagtggcaccgagtcggtgc

SEQ ID NO: 191 - DNA, 149 bp, synthetic construct; identical to SEQ ID NO: 163
  source 1..149: mol_type = other DNA

SEQ ID NO: 192 - DNA, 97 bp, synthetic construct; identical to SEQ ID NO: 190
  source 1..97: mol_type = other DNA

SEQ ID NO: 193 - DNA, 150 bp, synthetic construct; identical to SEQ ID NO: 165
  source 1..150: mol_type = other DNA

SEQ ID NO: 194 - DNA, 97 bp, synthetic construct; identical to SEQ ID NO: 190
  source 1..97: mol_type = other DNA

SEQ ID NO: 195 - DNA, 150 bp, synthetic construct; identical to SEQ ID NO: 167
  source 1..150: mol_type = other DNA

SEQ ID NO: 196 - DNA, 97 bp, synthetic construct; identical to SEQ ID NO: 190
  source 1..97: mol_type = other DNA

SEQ ID NO: 197 - DNA, 149 bp, synthetic construct; identical to SEQ ID NO: 169
  source 1..149: mol_type = other DNA

SEQ ID NO: 198 - DNA, 97 bp, synthetic construct; identical to SEQ ID NO: 190
  source 1..97: mol_type = other DNA

SEQ ID NO: 199 - DNA, 150 bp, synthetic construct; identical to SEQ ID NO: 171
  source 1..150: mol_type = other DNA

SEQ ID NO: 200 - DNA, 97 bp, synthetic construct; identical to SEQ ID NO: 190
  source 1..97: mol_type = other DNA

SEQ ID NO: 201 - DNA, 150 bp, synthetic construct; identical to SEQ ID NO: 173
  source 1..150: mol_type = other DNA

SEQ ID NO: 202 - DNA, 97 bp, synthetic construct; identical to SEQ ID NO: 190
  source 1..97: mol_type = other DNA

SEQ ID NO: 203 - DNA, 150 bp, synthetic construct; identical to SEQ ID NO: 161
  source 1..150: mol_type = other DNA

SEQ ID NO: 204 - DNA, 97 bp, synthetic construct
  source 1..97: mol_type = other DNA
gcgagacctccagcctgaggagttttagagctagaaatagcaagttaaaataaggctagtccgttatcaacttgaaaaagtggcaccgagtcggtgc

SEQ ID NO: 205 - DNA, 149 bp, synthetic construct; identical to SEQ ID NO: 163
  source 1..149: mol_type = other DNA

SEQ ID NO: 206 - DNA, 97 bp, synthetic construct; identical to SEQ ID NO: 204
  source 1..97: mol_type = other DNA

SEQ ID NO: 207 - DNA, 150 bp, synthetic construct; identical to SEQ ID NO: 165
  source 1..150: mol_type = other DNA

SEQ ID NO: 208 - DNA, 97 bp, synthetic construct; identical to SEQ ID NO: 204
  source 1..97: mol_type = other DNA

SEQ ID NO: 209 - DNA, 150 bp, synthetic construct; identical to SEQ ID NO: 167
  source 1..150: mol_type = other DNA

SEQ ID NO: 210 - DNA, 97 bp, synthetic construct; identical to SEQ ID NO: 204
  source 1..97: mol_type = other DNA

SEQ ID NO: 211 - DNA, 149 bp, synthetic construct; identical to SEQ ID NO: 169
  source 1..149: mol_type = other DNA

SEQ ID NO: 212 - DNA, 97 bp, synthetic construct; identical to SEQ ID NO: 204
  source 1..97: mol_type = other DNA

SEQ ID NO: 213 - DNA, 150 bp, synthetic construct; identical to SEQ ID NO: 171
  source 1..150: mol_type = other DNA

SEQ ID NO: 214 - DNA, 97 bp, synthetic construct; identical to SEQ ID NO: 204
  source 1..97: mol_type = other DNA

SEQ ID NO: 215 - DNA, 150 bp, synthetic construct; identical to SEQ ID NO: 173
  source 1..150: mol_type = other DNA

SEQ ID NO: 216 - DNA, 97 bp, synthetic construct; identical to SEQ ID NO: 204
  source 1..97: mol_type = other DNA

SEQ ID NO: 217 - DNA, 150 bp, synthetic construct; identical to SEQ ID NO: 161
  source 1..150: mol_type = other DNA

SEQ ID NO: 218 - DNA, 97 bp, synthetic construct
  source 1..97: mol_type = other DNA
gacaccgagacctccagcctggttttagagctagaaatagcaagttaaaataaggctagtccgttatcaacttgaaaaagtggcaccgagtcggtgc

SEQ ID NO: 219 - DNA, 149 bp, synthetic construct; identical to SEQ ID NO: 163
  source 1..149: mol_type = other DNA

SEQ ID NO: 220 - DNA, 97 bp, synthetic construct; identical to SEQ ID NO: 218
  source 1..97: mol_type = other DNA

SEQ ID NO: 221 - DNA, 150 bp, synthetic construct; identical to SEQ ID NO: 165
  source 1..150: mol_type = other DNA

SEQ ID NO: 222 - DNA, 97 bp, synthetic construct; identical to SEQ ID NO: 218
  source 1..97: mol_type = other DNA

SEQ ID NO: 223 - DNA, 150 bp, synthetic construct; identical to SEQ ID NO: 167
  source 1..150: mol_type = other DNA

SEQ ID NO: 224 - DNA, 97 bp, synthetic construct; identical to SEQ ID NO: 218
  source 1..97: mol_type = other DNA

SEQ ID NO: 225 - DNA, 149 bp, synthetic construct; identical to SEQ ID NO: 169
  source 1..149: mol_type = other DNA

SEQ ID NO: 226 - DNA, 97 bp, synthetic construct; identical to SEQ ID NO: 218
  source 1..97: mol_type = other DNA

SEQ ID NO: 227 - DNA, 150 bp, synthetic construct; identical to SEQ ID NO: 171
  source 1..150: mol_type = other DNA

SEQ ID NO: 228 - DNA, 97 bp, synthetic construct; identical to SEQ ID NO: 218
  source 1..97: mol_type = other DNA

SEQ ID NO: 229 - DNA, 150 bp, synthetic construct; identical to SEQ ID NO: 173
  source 1..150: mol_type = other DNA

SEQ ID NO: 230 - DNA, 97 bp, synthetic construct; identical to SEQ ID NO: 218
  source 1..97: mol_type = other DNA

SEQ ID NO: 231 - DNA, 150 bp, synthetic construct; identical to SEQ ID NO: 161
  source 1..150: mol_type = other DNA

SEQ ID NO: 232 - DNA, 97 bp, synthetic construct
  source 1..97: mol_type = other DNA
gagctagtcagacatggtggagttttagagctagaaatagcaagttaaaataaggctagtccgttatcaacttgaaaaagtggcaccgagtcggtgc

SEQ ID NO: 233 - DNA, 149 bp, synthetic construct; identical to SEQ ID NO: 163
  source 1..149: mol_type = other DNA

SEQ ID NO: 234 - DNA, 97 bp, synthetic construct; identical to SEQ ID NO: 232
  source 1..97: mol_type = other DNA

SEQ ID NO: 235 - DNA, 150 bp, synthetic construct; identical to SEQ ID NO: 165
  source 1..150: mol_type = other DNA

SEQ ID NO: 236 - DNA, 97 bp, synthetic construct; identical to SEQ ID NO: 232
  source 1..97: mol_type = other DNA

SEQ ID NO: 237 - DNA, 150 bp, synthetic construct; identical to SEQ ID NO: 167
  source 1..150: mol_type = other DNA

SEQ ID NO: 238 - DNA, 97 bp, synthetic construct; identical to SEQ ID NO: 232
  source 1..97: mol_type = other DNA

SEQ ID NO: 239 - DNA, 149 bp, synthetic construct; identical to SEQ ID NO: 169
  source 1..149: mol_type = other DNA

SEQ ID NO: 240 - DNA, 97 bp, synthetic construct; identical to SEQ ID NO: 232
  source 1..97: mol_type = other DNA

SEQ ID NO: 241 - DNA, 150 bp, synthetic construct; identical to SEQ ID NO: 171
  source 1..150: mol_type = other DNA

SEQ ID NO: 242 - DNA, 97 bp, synthetic construct; identical to SEQ ID NO: 232
  source 1..97: mol_type = other DNA

SEQ ID NO: 243 - DNA, 150 bp, synthetic construct; identical to SEQ ID NO: 173
  source 1..150: mol_type = other DNA

SEQ ID NO: 244 - DNA, 97 bp, synthetic construct; identical to SEQ ID NO: 232
  source 1..97: mol_type = other DNA

SEQ ID NO: 245 - DNA, 150 bp, synthetic construct; identical to SEQ ID NO: 161
  source 1..150: mol_type = other DNA

SEQ ID NO: 246 - DNA, 97 bp, synthetic construct
  source 1..97: mol_type = other DNA
gagctagctagtcagacatgggttttagagctagaaatagcaagttaaaataaggctagtccgttatcaacttgaaaaagtggcaccgagtcggtgc

SEQ ID NO: 247 - DNA, 149 bp, synthetic construct; identical to SEQ ID NO: 163
  source 1..149: mol_type = other DNA

SEQ ID NO: 248 - DNA, 97 bp, synthetic construct; identical to SEQ ID NO: 246
  source 1..97: mol_type = other DNA

SEQ ID NO: 249 - DNA, 150 bp, synthetic construct; identical to SEQ ID NO: 165
  source 1..150: mol_type = other DNA

SEQ ID NO: 250 - DNA, 97 bp, synthetic construct; identical to SEQ ID NO: 246
  source 1..97: mol_type = other DNA

SEQ ID NO: 251 - DNA, 150 bp, synthetic construct; identical to SEQ ID NO: 167
  source 1..150: mol_type = other DNA

SEQ ID NO: 252 - DNA, 97 bp, synthetic construct; identical to SEQ ID NO: 246
  source 1..97: mol_type = other DNA

SEQ ID NO: 253 - DNA, 149 bp, synthetic construct; identical to SEQ ID NO: 169
  source 1..149: mol_type = other DNA

SEQ ID NO: 254 - DNA, 97 bp, synthetic construct; identical to SEQ ID NO: 246
  source 1..97: mol_type = other DNA

SEQ ID NO: 255 - DNA, 150 bp, synthetic construct; identical to SEQ ID NO: 171
  source 1..150: mol_type = other DNA

SEQ ID NO: 256 - DNA, 97 bp, synthetic construct; identical to SEQ ID NO: 246
  source 1..97: mol_type = other DNA

SEQ ID NO: 257 - DNA, 150 bp, synthetic construct; identical to SEQ ID NO: 173
  source 1..150: mol_type = other DNA

SEQ ID NO: 258 - DNA, 97 bp, synthetic construct; identical to SEQ ID NO: 246
  source 1..97: mol_type = other DNA

SEQ ID NO: 259 - DNA, 150 bp, synthetic construct
  source 1..150: mol_type = other DNA
gcttgtcggctttagaagttagttttagagctagaaatagcaagttaaaataaggctagtccgttatcaacttgaaaaagtggcaccgagtcggtgcccctcgcctccttaaggcttgtcgacgacggcggtctccgtcgtcaggatcat

SEQ ID NO: 260 - DNA, 97 bp, synthetic construct; identical to SEQ ID NO: 162
  source 1..97: mol_type = other DNA

SEQ ID NO: 261 - DNA, 149 bp, synthetic construct
  source 1..149: mol_type = other DNA
gtcggctttagaagttaagggttttagagctagaaatagcaagttaaaataaggctagtccgttatcaacttgaaaaagtggcaccgagtcggtgccagccctcgcctcctggcttgtcgacgacggcggtctccgtcgtcaggatcat

SEQ ID NO: 262 - DNA, 97 bp, synthetic construct; identical to SEQ ID NO: 162
  source 1..97: mol_type = other DNA

SEQ ID NO: 263 - DNA, 150 bp, synthetic construct
  source 1..150: mol_type = other DNA
gctttagaagttaaggaggcggttttagagctagaaatagcaagttaaaataaggctagtccgttatcaacttgaaaaagtggcaccgagtcggtgcgaccccagccctcgcggcttgtcgacgacggcggtctccgtcgtcaggatcat

SEQ ID NO: 264 - DNA, 97 bp, synthetic construct; identical to SEQ ID NO: 162
  source 1..97: mol_type = other DNA

SEQ ID NO: 265 - DNA, 150 bp, synthetic construct
  source 1..150: mol_type = other DNA
gtttagaagttaaggaggcgagttttagagctagaaatagcaagttaaaataaggctagtccgttatcaacttgaaaaagtggcaccgagtcggtgcagaccccagccctcgggcttgtcgacgacggcggtctccgtcgtcaggatcat

SEQ ID NO: 266 - DNA, 97 bp, synthetic construct; identical to SEQ ID NO: 162
  source 1..97: mol_type = other DNA

SEQ ID NO: 267 - DNA, 149 bp, synthetic construct
  source 1..149: mol_type = other DNA
gaagttaaggaggcgagggcgttttagagctagaaatagcaagttaaaataaggctagtccgttatcaacttgaaaaagtggcaccgagtcggtgctgacagaccccagccggcttgtcgacgacggcggtctccgtcgtcaggatcat

SEQ ID NO: 268 - DNA, 97 bp, synthetic construct; identical to SEQ ID NO: 162
  source 1..97: mol_type = other DNA

SEQ ID NO: 269 - DNA, 150 bp, synthetic construct
  source 1..150: mol_type = other DNA
gaagttaaggaggcgagggctgttttagagctagaaatagcaagttaaaataaggctagtccgttatcaacttgaaaaagtggcaccgagtcggtgcctgacagaccccagcggcttgtcgacgacggcggtctccgtcgtcaggatcat

SEQ ID NO: 270 - DNA, 97 bp, synthetic construct; identical to SEQ ID NO: 162
  source 1..97: mol_type = other DNA

SEQ ID NO: 271 - DNA, 150 bp, synthetic construct
  source 1..150: mol_type = other DNA
gagttaaggaggcgagggctggttttagagctagaaatagcaagttaaaataaggctagtccgttatcaacttgaaaaagtggcaccgagtcggtgcactgacagaccccagggcttgtcgacgacggcggtctccgtcgtcaggatcat

SEQ ID NO: 272 - DNA, 97 bp, synthetic construct; identical to SEQ ID NO: 162
  source 1..97: mol_type = other DNA

SEQ ID NO: 273 - DNA, 150 bp, synthetic construct; identical to SEQ ID NO: 259
  source 1..150: mol_type = other DNA

SEQ ID NO: 274 - DNA, 96 bp, synthetic construct; identical to SEQ ID NO: 176
  source 1..96: mol_type = other DNA

SEQ ID NO: 275 - DNA, 149 bp, synthetic construct; identical to SEQ ID NO: 261
  source 1..149: mol_type = other DNA

SEQ ID NO: 276 - DNA, 96 bp, synthetic construct; identical to SEQ ID NO: 176
  source 1..96: mol_type = other DNA

SEQ ID NO: 277 - DNA, 150 bp, synthetic construct; identical to SEQ ID NO: 263
  source 1..150: mol_type = other DNA

SEQ ID NO: 278 - DNA, 96 bp, synthetic construct; identical to SEQ ID NO: 176
  source 1..96: mol_type = other DNA

SEQ ID NO: 279 - DNA, 150 bp, synthetic construct; identical to SEQ ID NO: 265
  source 1..150: mol_type = other DNA

SEQ ID NO: 280 - DNA, 96 bp, synthetic construct; identical to SEQ ID NO: 176
  source 1..96: mol_type = other DNA

SEQ ID NO: 281 - DNA, 149 bp, synthetic construct; identical to SEQ ID NO: 267
  source 1..149: mol_type = other DNA

SEQ ID NO: 282 - DNA, 96 bp, synthetic construct; identical to SEQ ID NO: 176
  source 1..96: mol_type = other DNA

SEQ ID NO: 283 - DNA, 150 bp, synthetic construct; identical to SEQ ID NO: 269
  source 1..150: mol_type = other DNA

SEQ ID NO: 284 - DNA, 96 bp, synthetic construct; identical to SEQ ID NO: 176
  source 1..96: mol_type = other DNA

SEQ ID NO: 285 - DNA, 150 bp, synthetic construct; identical to SEQ ID NO: 271
  source 1..150: mol_type = other DNA

SEQ ID NO: 286 - DNA, 96 bp, synthetic construct; identical to SEQ ID NO: 176
  source 1..96: mol_type = other DNA

SEQ ID NO: 287 - DNA, 150 bp, synthetic construct; identical to SEQ ID NO: 259
  source 1..150: mol_type = other DNA

SEQ ID NO: 288 - DNA, 97 bp, synthetic construct; identical to SEQ ID NO: 190
  source 1..97: mol_type = other DNA

SEQ ID NO: 289 - DNA, 149 bp, synthetic construct; identical to SEQ ID NO: 261
  source 1..149: mol_type = other DNA

SEQ ID NO: 290 - DNA, 97 bp, synthetic construct; identical to SEQ ID NO: 190
  source 1..97: mol_type = other DNA

SEQ ID NO: 291 - DNA, 150 bp, synthetic construct; identical to SEQ ID NO: 263
  source 1..150: mol_type = other DNA

SEQ ID NO: 292 - DNA, 97 bp, synthetic construct; identical to SEQ ID NO: 190
  source 1..97: mol_type = other DNA

SEQ ID NO: 293 - DNA, 150 bp, synthetic construct; identical to SEQ ID NO: 265
  source 1..150: mol_type = other DNA

SEQ ID NO: 294 - DNA, 97 bp, synthetic construct; identical to SEQ ID NO: 190
  source 1..97: mol_type = other DNA

SEQ ID NO: 295 - DNA, 149 bp, synthetic construct; identical to SEQ ID NO: 267
  source 1..149: mol_type = other DNA

SEQ ID NO: 296 - DNA, 97 bp, synthetic construct; identical to SEQ ID NO: 190
  source 1..97: mol_type = other DNA

SEQ ID NO: 297 - DNA, 150 bp, synthetic construct; identical to SEQ ID NO: 269
  source 1..150: mol_type = other DNA

SEQ ID NO: 298 - DNA, 97 bp, synthetic construct; identical to SEQ ID NO: 190
  source 1..97: mol_type = other DNA

SEQ ID NO: 299 - DNA, 150 bp, synthetic construct; identical to SEQ ID NO: 271
  source 1..150: mol_type = other DNA

SEQ ID NO: 300 - DNA, 97 bp, synthetic construct; identical to SEQ ID NO: 190
  source 1..97: mol_type = other DNA

SEQ ID NO: 301 - DNA, 150 bp, synthetic construct; identical to SEQ ID NO: 259
  source 1..150: mol_type = other DNA

SEQ ID NO: 302 - DNA, 97 bp, synthetic construct; identical to SEQ ID NO: 204
  source 1..97: mol_type = other DNA

SEQ ID NO: 303 - DNA, 149 bp, synthetic construct; identical to SEQ ID NO: 261
  source 1..149: mol_type = other DNA

SEQ ID NO: 304 - DNA, 97 bp, synthetic construct; identical to SEQ ID NO: 204
  source 1..97: mol_type = other DNA

SEQ ID NO: 305 - DNA, 150 bp, synthetic construct; identical to SEQ ID NO: 263
  source 1..150: mol_type = other DNA

SEQ ID NO: 306 - DNA, 97 bp, synthetic construct; identical to SEQ ID NO: 204
  source 1..97: mol_type = other DNA

SEQ ID NO: 307 - DNA, 150 bp, synthetic construct; identical to SEQ ID NO: 265
  source 1..150: mol_type = other DNA

SEQ ID NO: 308 - DNA, 97 bp, synthetic construct; identical to SEQ ID NO: 204
  source 1..97: mol_type = other DNA

SEQ ID NO: 309 - DNA, 149 bp, synthetic construct; identical to SEQ ID NO: 267
  source 1..149: mol_type = other DNA

SEQ ID NO: 310 - DNA, 97 bp, synthetic construct; identical to SEQ ID NO: 204
  source 1..97: mol_type = other DNA

SEQ ID NO: 311 - DNA, 150 bp, synthetic construct; identical to SEQ ID NO: 269
  source 1..150: mol_type = other DNA

SEQ ID NO: 312 - DNA, 97 bp, synthetic construct; identical to SEQ ID NO: 204
  source 1..97: mol_type = other DNA

SEQ ID NO: 313 - DNA, 150 bp, synthetic construct; identical to SEQ ID NO: 271
  source 1..150: mol_type = other DNA

SEQ ID NO: 314 - DNA, 97 bp, synthetic construct; identical to SEQ ID NO: 204
  source 1..97: mol_type = other DNA

SEQ ID NO: 315 - DNA, 150 bp, synthetic construct; identical to SEQ ID NO: 259
  source 1..150: mol_type = other DNA

SEQ ID NO: 316 - DNA, 97 bp, synthetic construct; identical to SEQ ID NO: 218
  source 1..97: mol_type = other DNA

SEQ ID NO: 317 - DNA, 149 bp, synthetic construct; identical to SEQ ID NO: 261
  source 1..149: mol_type = other DNA

SEQ ID NO: 318 - DNA, 97 bp, synthetic construct; identical to SEQ ID NO: 218
  source 1..97: mol_type = other DNA

SEQ ID NO: 319 - DNA, 150 bp, synthetic construct; identical to SEQ ID NO: 263
  source 1..150: mol_type = other DNA

SEQ ID NO: 320 - DNA, 97 bp, synthetic construct; identical to SEQ ID NO: 218
  source 1..97: mol_type = other DNA

SEQ ID NO: 321 - DNA, 150 bp, synthetic construct; identical to SEQ ID NO: 265
  source 1..150: mol_type = other DNA

SEQ ID NO: 322 - DNA, 97 bp, synthetic construct; identical to SEQ ID NO: 218
  source 1..97: mol_type = other DNA

SEQ ID NO: 323 - DNA, 149 bp, synthetic construct; identical to SEQ ID NO: 267
  source 1..149: mol_type = other DNA

SEQ ID NO: 324 - DNA, 97 bp, synthetic construct; identical to SEQ ID NO: 218
  source 1..97: mol_type = other DNA

SEQ ID NO: 325 - DNA, 150 bp, synthetic construct; identical to SEQ ID NO: 269
  source 1..150: mol_type = other DNA

SEQ ID NO: 326 - DNA, 97 bp, synthetic construct; identical to SEQ ID NO: 218
  source 1..97: mol_type = other DNA

SEQ ID NO: 327 - DNA, 150 bp, synthetic construct; identical to SEQ ID NO: 271
  source 1..150: mol_type = other DNA

SEQ ID NO: 328 - DNA, 97 bp, synthetic construct; identical to SEQ ID NO: 218
  source 1..97: mol_type = other DNA

SEQ ID NO: 329 - DNA, 150 bp, synthetic construct; identical to SEQ ID NO: 259
  source 1..150: mol_type = other DNA

SEQ ID NO: 330 - DNA, 97 bp, synthetic construct; identical to SEQ ID NO: 232
  source 1..97: mol_type = other DNA

SEQ ID NO: 331 - DNA, 149 bp, synthetic construct; identical to SEQ ID NO: 261
  source 1..149: mol_type = other DNA

SEQ ID NO: 332 - DNA, 97 bp, synthetic construct; identical to SEQ ID NO: 232
  source 1..97: mol_type = other DNA

SEQ ID NO: 333 - DNA, 150 bp, synthetic construct; identical to SEQ ID NO: 263
  source 1..150: mol_type = other DNA

SEQ ID NO: 334 - DNA, 97 bp, synthetic construct; identical to SEQ ID NO: 232
  source 1..97: mol_type = other DNA

SEQ ID NO: 335 - DNA, 150 bp, synthetic construct; identical to SEQ ID NO: 265
  source 1..150: mol_type = other DNA

SEQ ID NO: 336 - DNA, 97 bp, synthetic construct; identical to SEQ ID NO: 232
  source 1..97: mol_type = other DNA

SEQ ID NO: 337 - DNA, 149 bp, synthetic construct; identical to SEQ ID NO: 267
  source 1..149: mol_type = other DNA

SEQ ID NO: 338 - DNA, 97 bp, synthetic construct; identical to SEQ ID NO: 232
  source 1..97: mol_type = other DNA

SEQ ID NO: 339 - DNA, 150 bp, synthetic construct; identical to SEQ ID NO: 269
  source 1..150: mol_type = other DNA

SEQ ID NO: 340 - DNA, 97 bp, synthetic construct; identical to SEQ ID NO: 232
  source 1..97: mol_type = other DNA

SEQ ID NO: 341 - DNA, 150 bp, synthetic construct; identical to SEQ ID NO: 271
  source 1..150: mol_type = other DNA

SEQ ID NO: 342 - DNA, 97 bp, synthetic construct; identical to SEQ ID NO: 232
  source 1..97: mol_type = other DNA

SEQ ID NO: 343 - DNA, 150 bp, synthetic construct; identical to SEQ ID NO: 259
  source 1..150: mol_type = other DNA

SEQ ID NO: 344 - DNA, 97 bp, synthetic construct; identical to SEQ ID NO: 246
  source 1..97: mol_type = other DNA

SEQ ID NO: 345 - DNA, 149 bp, synthetic construct; identical to SEQ ID NO: 261
  source 1..149: mol_type = other DNA

SEQ ID NO: 346 - DNA, 97 bp, synthetic construct; identical to SEQ ID NO: 246
  source 1..97: mol_type = other DNA

SEQ ID NO: 347 - DNA, 150 bp, synthetic construct; identical to SEQ ID NO: 263
  source 1..150: mol_type = other DNA

SEQ ID NO: 348 - DNA, 97 bp, synthetic construct; identical to SEQ ID NO: 246
  source 1..97: mol_type = other DNA

SEQ ID NO: 349 - DNA, 150 bp, synthetic construct; identical to SEQ ID NO: 265
  source 1..150: mol_type = other DNA

SEQ ID NO: 350 - DNA, 97 bp, synthetic construct; identical to SEQ ID NO: 246
  source 1..97: mol_type = other DNA

SEQ ID NO: 351 - DNA, 149 bp, synthetic construct; identical to SEQ ID NO: 267
  source 1..149: mol_type = other DNA

SEQ ID NO: 352 - DNA, 97 bp, synthetic construct; identical to SEQ ID NO: 246
  source 1..97: mol_type = other DNA

SEQ ID NO: 353 - DNA, 150 bp, synthetic construct; identical to SEQ ID NO: 269
  source 1..150: mol_type = other DNA

SEQ ID NO: 354 - DNA, 97 bp, synthetic construct; identical to SEQ ID NO: 246
  source 1..97: mol_type = other DNA

SEQ ID NO: 355 - DNA, 150 bp, synthetic construct; identical to SEQ ID NO: 271
  source 1..150: mol_type = other DNA

SEQ ID NO: 356 - DNA, 97 bp, synthetic construct; identical to SEQ ID NO: 246
  source 1..97: mol_type = other DNA

SEQ ID NO: 357 - DNA, 152 bp, synthetic construct
  source 1..152: mol_type = other DNA
agttaaggaggcgagggctggttttagagctagaaatagcaagttaaaataaggctagtccgttatcaacttgaaaaagtggcaccgagtcggtgcccggatgatcctgacgacggagaccgccgtcgtcgacaagccggccccctcgcctc

SEQ ID NO: 358 - DNA, 152 bp, synthetic construct
  source 1..152: mol_type = other DNA
ggaaggtccgcagagaagctgttttagagctagaaatagcaagttaaaataaggctagtccgttatcaacttgaaaaagtggcaccgagtcggtgcggccggcttgtcgacgacggcggtctccgtcgtcaggatcatccggttctctgcgg

SEQ ID NO: 359 - DNA, 144 bp, synthetic construct
  source 1..144: mol_type = other DNA
agttaaggaggcgagggctggttttagagctagaaatagcaagttaaaataaggctagtccgttatcaacttgaaaaagtggcaccgagtcggtgcatgatcctgacgacggagaccgccgtcgtcgacaagccccctcgcctc

SEQ ID NO: 360 - DNA, 144 bp, synthetic construct
  source 1..144: mol_type = other DNA
aggaaggtccgcagagaagcgttttagagctagaaatagcaagttaaaataaggctagtccgttatcaacttgaaaaagtggcaccgagtcggtgcggcttgtcgacgacggcggtctccgtcgtcaggatcattctctgcgga

SEQ ID NO: 361 - DNA, 144 bp, synthetic construct; identical to SEQ ID NO: 359
  source 1..144: mol_type = other DNA

SEQ ID NO: 362 - DNA, 144 bp, synthetic construct
  source 1..144: mol_type = other DNA
acaccgagacctccagcctggttttagagctagaaatagcaagttaaaataaggctagtccgttatcaacttgaaaaagtggcaccgagtcggtgcggcttgtcgacgacggcggtctccgtcgtcaggatcatgctggaggtc

SEQ ID NO: 363 - DNA, 151 bp, synthetic construct
  source 1..151: mol_type = other DNA
gcgtattgcctggaggatgggttttagagctagaaatagcaagttaaaataaggctagtccgttatcaacttgaaaaagtggcaccgagtcggtgcccggcttgtcgacgacggcggtctccgtcgtcaggatcatcctcctccaggcaat

SEQ ID NO: 364 - DNA, 152 bp, synthetic construct
  source 1..152: mol_type = other DNA
gtattggccacctctgagagtgttttagagctagaaatagcaagttaaaataaggctagtccgttatcaacttgaaaaagtggcaccgagtcggtgcggatgatcctgacgacggagaccgccgtcgtcgacaagccggctcagaggtggcc

SEQ ID NO: 365 - DNA, 147 bp, synthetic construct
  source 1..147: mol_type = other DNA
gcgtattgcctggaggatgggttttagagctagaaatagcaagttaaaataaggctagtccgttatcaacttgaaaaagtggcaccgagtcggtgcatgatcctgacgacggagaccgccgtcgtcgacaagcctcctccaggcaat

SEQ ID NO: 366 - DNA, 148 bp, synthetic construct
  source 1..148: mol_type = other DNA
gtattggccacctctgagagtgttttagagctagaaatagcaagttaaaataaggctagtccgttatcaacttgaaaaagtggcaccgagtcggtgcggcttgtcgacgacggcggtctccgtcgtcaggatcatctcagaggtggcc

SEQ ID NO: 367 - DNA, 151 bp, synthetic construct
  source 1..151: mol_type = other DNA
gcgtattgcctggaggatgggttttagagctagaaatagcaagttaaaataaggctagtccgttatcaacttgaaaaagtggcaccgagtcggtgcggccggcttgtcgacgacggcggtctccgtcgtcaggatcatccggtcctccagg

SEQ ID NO: 368 - DNA, 152 bp, synthetic construct
  source 1..152: mol_type = other DNA
gtattggccacctctgagagtgttttagagctagaaatagcaagttaaaataaggctagtccgttatcaacttgaaaaagtggcaccgagtcggtgcggccggcttgtcgacgacggcggtctccgtcgtcaggatcatccggctcagaggt

SEQ ID NO: 369 - DNA, 147 bp, synthetic construct
  source 1..147: mol_type = other DNA
gcgtattgcctggaggatgggttttagagctagaaatagcaagttaaaataaggctagtccgttatcaacttgaaaaagtggcaccgagtcggtgcggcttgtcgacgacggcggtctccgtcgtcaggatcattcctccaggcaat

SEQ ID NO: 370 - DNA, 148 bp, synthetic construct
  source 1..148: mol_type = other DNA
gtattggccacctctgagagtgttttagagctagaaatagcaagttaaaataaggctagtccgttatcaacttgaaaaagtggcaccgagtcggtgcatgatcctgacgacggagaccgccgtcgtcgacaagccctcagaggtggcc

SEQ ID NO: 371 - DNA, 189 bp, synthetic construct
  source 1..189: mol_type = other DNA
gcgtattgcctggaggatgggttttagagctagaaatagcaagttaaaataaggctagtccgttatcaacttgaaaaagtggcaccgagtcggtgcgaaccacgcggcgaatgccggcgtccgccccggatgatcctgacgacggagaccgccgtcgtcgacaagccggcctcctccaggcaatacgcg

SEQ ID NO: 372 - DNA, 97 bp, synthetic construct
  source 1..97: mol_type = other DNA
gagccgagcacgaggggatacgttttagagctagaaatagcaagttaaaataaggctagtccgttatcaacttgaaaaagtggcaccgagtcggtgc

SEQ ID NO: 373 - DNA, 152 bp, synthetic construct; identical to SEQ ID NO: 357
  source 1..152: mol_type = other DNA

SEQ ID NO: 374 - DNA, 152 bp, synthetic construct; identical to SEQ ID NO: 358
  source 1..152: mol_type = other DNA

SEQ ID NO: 375 - DNA, 144 bp, synthetic construct; identical to SEQ ID NO: 359
  source 1..144: mol_type = other DNA

SEQ ID NO: 376 - DNA, 144 bp, synthetic construct; identical to SEQ ID NO: 362
  source 1..144: mol_type = other DNA

SEQ ID NO: 377 - DNA, 144 bp, synthetic construct
  source 1..144: mol_type = other DNA
agttaaggaggcgagggctggttttagagctagaaatagcaagttaaaataaggctagtccgttatcaacttgaaaaagtggcaccgagtcggtgcggcttgtcgacgacggcggtctccgtcgtcaggatcatccctcgcctc

SEQ ID NO: 378 - DNA, 144 bp, synthetic construct
  source 1..144: mol_type = other DNA
acaccgagacctccagcctggttttagagctagaaatagcaagttaaaataaggctagtccgttatcaacttgaaaaagtggcaccgagtcggtgcatgatcctgacgacggagaccgccgtcgtcgacaagccgctggaggtc

SEQ ID NO: 379 - DNA, 144 bp, synthetic construct
  source 1..144: mol_type = other DNA
aagttaaggaggcgagggctgttttagagctagaaatagcaagttaaaataaggctagtccgttatcaacttgaaaaagtggcaccgagtcggtgcggcttgtcgacgacggcggtctccgtcgtcaggatcatcctcgcctcc

SEQ ID NO: 380 - DNA, 144 bp, synthetic construct
  source 1..144: mol_type = other DNA
ggaaggtccgcagagaagctgttttagagctagaaatagcaagttaaaataaggctagtccgttatcaacttgaaaaagtggcaccgagtcggtgcatgatcctgacgacggagaccgccgtcgtcgacaagccttctctgcgg

SEQ ID NO: 381 - DNA, 152 bp, synthetic construct; identical to SEQ ID NO: 357
  source 1..152: mol_type = other DNA

SEQ ID NO: 382 - DNA, 152 bp, synthetic construct
  source 1..152: mol_type = other DNA
agctagtcagacatggtggagttttagagctagaaatagcaagttaaaataaggctagtccgttatcaacttgaaaaagtggcaccgagtcggtgcggccggcttgtcgacgacggcggtctccgtcgtcaggatcatccggaccatgtctg

SEQ ID NO: 383 - DNA, 144 bp, synthetic construct
  source 1..144: mol_type = other DNA
gtcggctttagaagttaagggttttagagctagaaatagcaagttaaaataaggctagtccgttatcaacttgaaaaagtggcaccgagtcggtgcatgatcctgacgacggagaccgccgtcgtcgacaagcctaacttctaa

SEQ ID NO: 384 - DNA, 144 bp, synthetic construct
  source 1..144: mol_type = other DNA
ggaaggtccgcagagaagctgttttagagctagaaatagcaagttaaaataaggctagtccgttatcaacttgaaaaagtggcaccgagtcggtgcggcttgtcgacgacggcggtctccgtcgtcaggatcatttctctgcgg

SEQ ID NO: 385 - DNA, 144 bp, synthetic construct; identical to SEQ ID NO: 359
  source 1..144: mol_type = other DNA

SEQ ID NO: 386 - DNA, 144 bp, synthetic construct; identical to SEQ ID NO: 384
  source 1..144: mol_type = other DNA

SEQ ID NO: 387 - DNA, 144 bp, synthetic construct; identical to SEQ ID NO: 379
  source 1..144: mol_type = other DNA

SEQ ID NO: 388 - DNA, 144 bp, synthetic construct; identical to SEQ ID NO: 378
  source 1..144: mol_type = other DNA

SEQ ID NO: 389 - DNA, 144 bp, synthetic construct; identical to SEQ ID NO: 377
  source 1..144: mol_type = other DNA

SEQ ID NO: 390 - DNA, 144 bp, synthetic construct; identical to SEQ ID NO: 380
  source 1..144: mol_type = other DNA

SEQ ID NO: 391 - DNA, 144 bp, synthetic construct; identical to SEQ ID NO: 377
  source 1..144: mol_type = other DNA

SEQ ID NO: 392 - DNA, 144 bp, synthetic construct
  source 1..144: mol_type = other DNA
aggaaggtccgcagagaagcgttttagagctagaaatagcaagttaaaataaggctagtccgttatcaacttgaaaaagtggcaccgagtcggtgcatgatcctgacgacggagaccgccgtcgtcgacaagcctctctgcgga

SEQ ID NO: 393 - DNA, 155 bp, synthetic construct
  source 1..155: mol_type = other DNA
gcgttttacccggagcatgggttttagagctagaaatagcaagttaaaataaggctagtccgttatcaacttgaaaaagtggcaccgagtcggtgcccggatgatcctgacgacggagaccgccgtcgtcgacaagccggcctgctccgggtaaa

SEQ ID NO: 394 - DNA, 156 bp, synthetic construct
  source 1..156: mol_type = other DNA
gtactggccacctccgagagtgttttagagctagaaatagcaagttaaaataaggctagtccgttatcaacttgaaaaagtggcaccgagtcggtgcggccggcttgtcgacgacggcggtctccgtcgtcaggatcatccggctcggaggtggcc

SEQ ID NO: 395 - DNA, 52 bp, synthetic construct
  source 1..52: mol_type = other DNA
gtggtttgtctggtcaaccaccgcggtctcagtggtgtacggtacaaaccca

SEQ ID NO: 396 - DNA, 48 bp, synthetic construct
  source 1..48: mol_type = other DNA
ggtttgtctggtcaaccaccgcggtctcagtggtgtacggtacaaacc

SEQ ID NO: 397 - DNA, 76 bp, synthetic construct
  source 1..76: mol_type = other DNA
gttttagagctagaaatagcaagttaaaataaggctagtccgttatcaacttgaaaaagtggcaccgagtcggtgc

SEQ ID NO: 398 - protein, 1368 aa, Streptococcus pyogenes [sequence omitted]

SEQ ID NO: 399 - protein, 1368 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 400 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 401 - protein, 677 aa, unidentified
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSP

SEQ ID NO: 402 - protein, 497 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLD

SEQ ID NO: 403 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 404 - protein, 497 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLD

SEQ ID NO: 405 - protein, 500 aa, unidentified
SRALVVIRLSRVTDATTSPERQLESCQQLCAQRGWDVVGVAEDLDVSGAVDPFDRKRRPNLARWLAFEEQPFDVIVAYRVDRLTRSIRHLQQLVHWAEDHKKLVVSATEAHFDTTTPFAAVVIALMGTVAQMELEAIKERNRSAAHFNIRAGKYRGSLPPWGYLPTRVDGEWRLVPDPVQRERILEVYHRVVDNHEPLHLVAHDLNRRGVLSPKDYFAQLQGREPQGREWSATALKRSMISEAMLGYATLNGKTVRDDDGAPLVRAEPILTREQLEALRAELVKTSRAKPAVSTPSLLLRVLFCAVCGEPAYKFAGGGRKHPRYRCRSMGFPKHCGNGTVAMAEWDAFCEEQVLDLLGDAERLEKVWVAGSDSAVELAEVNAELVDLTSLIGSPAYRAGSPQREALDARIAALAARQEELEGLEARPSGWEWRETGQRFGDWWREQDTAAKNTWLRSMNVRLTFDVRGGLTRTIDFGDLQEYEQHLRLGSVVERLHTGMS

SEQ ID NO: 406 - protein, 2265 aa, synthetic construct
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSAGGGGSGGGGSGGGGSGMKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFEPKKKRKV

SEQ ID NO: 407 - DNA, 34 bp, synthetic construct
  source 1..34: mol_type = other DNA
ataacttcgtataatgtatgctatacgaacggta

SEQ ID NO: 408 - DNA, 34 bp, synthetic construct
  source 1..34: mol_type = other DNA
taccgttcgtataatgtatgctatacgaagttat

SEQ ID NO: 409 - DNA, 46 bp, synthetic construct
  source 1..46: mol_type = other DNA
ggccggcttgtcgacgacggcggtctccgtcgtcaggatcatccgg

SEQ ID NO: 410 - DNA, 46 bp, synthetic construct
  source 1..46: mol_type = other DNA
ccggatgatcctgacgacggagaccgccgtcgtcgacaagccggcc

SEQ ID NO: 411 - DNA, 38 bp, synthetic construct
  source 1..38: mol_type = other DNA
ggcttgtcgacgacggcgttctccgtcgtcaggatcat

SEQ ID NO: 412 - DNA, 52 bp, synthetic construct
  source 1..52: mol_type = other DNA
gtggtttgtctggtcaaccaccgcgttctcagtggtgtacggtacaaaccca

SEQ ID NO: 413 - DNA, 38 bp, synthetic construct
  source 1..38: mol_type = other DNA
ggcttgtcgacgacggcgaactccgtcgtcaggatcat

SEQ ID NO: 414 - DNA, 52 bp, synthetic construct
  source 1..52: mol_type = other DNA
gtggtttgtctggtcaaccaccgcgaactcagtggtgtacggtacaaaccca

SEQ ID NO: 415 - DNA, 38 bp, synthetic construct
  source 1..38: mol_type = other DNA
ggcttgtcgacgacggcgccctccgtcgtcaggatcat

SEQ ID NO: 416 - DNA, 52 bp, synthetic construct
  source 1..52: mol_type = other DNA
gtggtttgtctggtcaaccaccgcgccctcagtggtgtacggtacaaaccca

SEQ ID NO: 417 - DNA, 38 bp, synthetic construct
  source 1..38: mol_type = other DNA
ggcttgtcgacgacggcgggctccgtcgtcaggatcat

SEQ ID NO: 418 - DNA, 52 bp, synthetic construct
  source 1..52: mol_type = other DNA
gtggtttgtctggtcaaccaccgcgggctcagtggtgtacggtacaaaccca

SEQ ID NO: 419 - DNA, 38 bp, synthetic construct
  source 1..38: mol_type = other DNA
ggcttgtcgacgacggcgtgctccgtcgtcaggatcat

SEQ ID NO: 420 - DNA, 52 bp, synthetic construct
  source 1..52: mol_type = other DNA
gtggtttgtctggtcaaccaccgcgtgctcagtggtgtacggtacaaaccca

SEQ ID NO: 421 - DNA, 38 bp, synthetic construct
  source 1..38: mol_type = other DNA
ggcttgtcgacgacggcggtctccgtcgtcaggatcat

SEQ ID NO: 422 - DNA, 38 bp, synthetic construct
  source 1..38: mol_type = other DNA
ggcttgtcgacgacggcgctctccgtcgtcaggatcat

SEQ ID NO: 423 - DNA, 52 bp, synthetic construct
  source 1..52: mol_type = other DNA
gtggtttgtctggtcaaccaccgcgctctcagtggtgtacggtacaaaccca

SEQ ID NO: 424 - DNA, 38 bp, synthetic construct
  source 1..38: mol_type = other DNA
ggcttgtcgacgacggcgcactccgtcgtcaggatcat

SEQ ID NO: 425 - DNA, 52 bp, synthetic construct
  source 1..52: mol_type = other DNA
gtggtttgtctggtcaaccaccgcgcactcagtggtgtacggtacaaaccca

SEQ ID NO: 426 - DNA, 38 bp, synthetic construct
  source 1..38: mol_type = other DNA
ggcttgtcgacgacggcgtcctccgtcgtcaggatcat

SEQ ID NO: 427 - DNA, 52 bp, synthetic construct
  source 1..52: mol_type = other DNA
gtggtttgtctggtcaaccaccgcgtcctcagtggtgtacggtacaaaccca

SEQ ID NO: 428 - DNA, 38 bp, synthetic construct
  source 1..38: mol_type = other DNA
ggcttgtcgacgacggcggactccgtcgtcaggatcat

SEQ ID NO: 429 - DNA, 52 bp, synthetic construct
  source 1..52: mol_type = other DNA
gtggtttgtctggtcaaccaccgcggactcagtggtgtacggtacaaaccca

SEQ ID NO: 430 - DNA, 38 bp, synthetic construct
  source 1..38: mol_type = other DNA
ggcttgtcgacgacggcgagctccgtcgtcaggatcat

SEQ ID NO: 431 - DNA, 52 bp, synthetic construct
  source 1..52: mol_type = other DNA
gtggtttgtctggtcaaccaccgcgagctcagtggtgtacggtacaaaccca

SEQ ID NO: 432 - DNA, 38 bp, synthetic construct
  source 1..38: mol_type = other DNA
ggcttgtcgacgacggcgacctccgtcgtcaggatcat

SEQ ID NO: 433 - DNA, 52 bp, synthetic construct
  source 1..52: mol_type = other DNA
gtggtttgtctggtcaaccaccgcgacctcagtggtgtacggtacaaaccca

SEQ ID NO: 434 - DNA, 38 bp, synthetic construct
  source 1..38: mol_type = other DNA
ggcttgtcgacgacggcgatctccgtcgtcaggatcat

SEQ ID NO: 435 - DNA, 52 bp, synthetic construct
  source 1..52: mol_type = other DNA
gtggtttgtctggtcaaccaccgcgatctcagtggtgtacggtacaaaccca

SEQ ID NO: 436 - DNA, 38 bp, synthetic construct
  source 1..38: mol_type = other DNA
ggcttgtcgacgacggcggcctccgtcgtcaggatcat

SEQ ID NO: 437 - DNA, 52 bp, synthetic construct
  source 1..52: mol_type = other DNA
gtggtttgtctggtcaaccaccgcggcctcagtggtgtacggtacaaaccca

SEQ ID NO: 438 - DNA, 38 bp, synthetic construct
  source 1..38: mol_type = other DNA
ggcttgtcgacgacggcgcgctccgtcgtcaggatcat

SEQ ID NO: 439 - DNA, 52 bp, synthetic construct
  source 1..52: mol_type = other DNA
gtggtttgtctggtcaaccaccgcgcgctcagtggtgtacggtacaaaccca

SEQ ID NO: 440 - DNA, 38 bp, synthetic construct
  source 1..38: mol_type = other DNA
ggcttgtcgacgacggcgtactccgtcgtcaggatcat

SEQ ID NO: 441 - DNA, 52 bp, synthetic construct
  source 1..52: mol_type = other DNA
gtggtttgtctggtcaaccaccgcgtactcagtggtgtacggtacaaaccca

SEQ ID NO: 442 - DNA, 45 bp, synthetic construct
  source 1..45: mol_type = other DNA
tgcgggtgccagggcgtgcccttgggctccccgggcgcgtactcc

SEQ ID NO: 443 - DNA, 42 bp, synthetic construct
  source 1..42: mol_type = other DNA
gtgccccaactggggtaacctttgagttctctcagttggggg

SEQ ID NO: 444 - DNA, 57 bp, synthetic construct
  source 1..57: mol_type = other DNA
gcgcccaagttgcccatgaccatgccgaagcagtggtagaagggcaccggcagacac

SEQ ID NO: 445 - DNA, 70 bp, synthetic construct
  source 1..70: mol_type = other DNA
aggcatgttccccaaagcgataccacttgaagcagtggtactgcttgtgggtacactctgcgggtgatga

SEQ ID NO: 446 - DNA, 60 bp, synthetic construct
  source 1..60: mol_type = other DNA
gtccttgaccaggtttttgacgaaagtgatccagatgatccagctccacaccccgaacgc

SEQ ID NO: 447 - DNA, 63 bp, synthetic construct
  source 1..63: mol_type = other DNA
ggtgctgggttgttgtctctggacagtgatccatgggaaactactcagcaccaccaatgttcc

SEQ ID NO: 448 - DNA, 50 bp, synthetic construct
  source 1..50: mol_type = other DNA
tcggccggcttgtcgacgacggcggtctccgtcgtcaggatcatccgggc

SEQ ID NO: 449 - DNA, 58 bp, synthetic construct
  source 1..58: mol_type = other DNA
gtcgtggtttgtctggtcaaccaccgcggtctcagtggtgtacggtacaaaccccgac

SEQ ID NO: 450 - DNA, 46 bp, synthetic construct
  source 1..46: mol_type = other DNA
gatcagctccgcgggcaagaccttctccttcacggggtggaaggtc

SEQ ID NO: 451 - DNA, 67 bp, synthetic construct
  source 1..67: mol_type = other DNA
tcaaccccgttccagcccaacagtgttagtctttgctcttacccagttgggcgggatagcctgcccg

SEQ ID NO: 452 - DNA, 57 bp, synthetic construct
  source 1..57: mol_type = other DNA
aacgattttcaaaggatcactgaatcaaaagtattgctcatccacgcgaaatttttc

SEQ ID NO: 453 - DNA, 57 bp, synthetic construct
  source 1..57: mol_type = other DNA
aatattttaggtatatgattttgtttattagtgtaaataacactatgtacctaaaat

SEQ ID NO: 454 - DNA, 53 bp, synthetic construct
  source 1..53: mol_type = other DNA
tgtaaaggagactgataatggcatgtacaactatactcgtcggtaaaaaggca

SEQ ID NO: 455 - DNA, 52 bp, synthetic construct
  source 1..52: mol_type = other DNA
taaaaaaatacagcgtttttcatgtacaactatactagttgtagtgcctaaa

SEQ ID NO: 456 - DNA, 56 bp, synthetic construct
  source 1..56: mol_type = other DNA
gagcgccggatcagggagtggacggcctgggagcgctacacgctgtggctgcggtc

SEQ ID NO: 457 - DNA, 56 bp, synthetic construct
  source 1..56: mol_type = other DNA
ccctaatacgcaagtcgataactctcctgggagcgttgacaacttgcgcaccctga

SEQ ID NO: 458 - DNA, 68 bp, synthetic construct
  source 1..68: mol_type = other DNA
tctcgtggtggtggaaggtgttggtgcggggttggccgtggtcgaggtggggtggtggtagccattcg

SEQ ID NO: 459 - DNA, 69 bp, synthetic construct
  source 1..69: mol_type = other DNA
gcacaggtgtagtgtatctcacaggtccacggttggccgtggactgctgaagaacattccacgccagga

SEQ ID NO: 460 - DNA, 65 bp, synthetic construct
  source 1..65: mol_type = other DNA
agtgcagcatgtcattaatatcagtacagataaagctgtatctcctgtgaacacaatgggtgcca

SEQ ID NO: 461 - DNA, 55 bp, synthetic construct
  source 1..55: mol_type = other DNA
aaagtagtaagtatcttaaaaaacagataaagctgtatattaagatacttactac

SEQ ID NO: 462 - DNA, 54 bp, synthetic construct
  source 1..54: mol_type = other DNA
tgataattgccaacacaattaacatctcaatcaaggtaaatgctttttcgtttt

SEQ ID NO: 463 - DNA, 54 bp, synthetic construct
  source 1..54: mol_type = other DNA
aattgcgagtttttatttcgtttatttcaattaaggtaactaaaaaactccttt

SEQ ID NO: 464 - DNA, 68 bp, synthetic construct
  source 1..68: mol_type = other DNA
aaggtagcgtcaacgataggtgtaactgtcgtgtttgtaacggtacttccaacagctggcgtttcagt

SEQ ID NO: 465 - DNA, 68 bp, synthetic construct
  source 1..68: mol_type = other DNA
tagttttaaagttggttattagttactgtgatatttatcacggtacccaataaccaatgaatatttga

SEQ ID NO: 466 - DNA, 57 bp, synthetic construct
  source 1..57: mol_type = other DNA
tgtaactttttcggatcaagctatgaaggacgcaaagagggaactaaacacttaatt

SEQ ID NO: 467 - DNA, 57 bp, synthetic construct
  source 1..57: mol_type = other DNA
ttgtttagttcctcgttttctctcgttggaagaagaagaaacgagaaactaaaatta

SEQ ID NO: 468 - DNA, 63 bp, synthetic construct
  source 1..63: mol_type = other DNA
caacctgttgacatgtttccacagacaactcacgtggaggtagtcacggcttttacgttagtt

SEQ ID NO: 469 - DNA, 61 bp, synthetic construct
  source 1..61: mol_type = other DNA
gagaatactgttgaacaatgaaaaactaggcatgtagaagttgtttgtgcactaactttaa

SEQ ID NO: 470 - DNA, 120 bp, synthetic construct
  source 1..120: mol_type = other DNA
acaggtcaacacatcgcagttatcgaacaatcttcgaaaatgtatggaggcacttgtatcaatataggatgtataccttcgaagacacttgtacatgatggattagaaggcaaatccttt

SEQ ID NO: 471 - DNA, 120 bp, synthetic construct
  source 1..120: mol_type = other DNA
caaaataaaaaacattgatttttattaacttcttttgtgcggaactacgaacagttcattaatacgaagtgtacaaacttccatacaaaaataaccacgacaattaagacgtggtttcta

SEQ ID NO: 472 - DNA, 17 bp, synthetic construct
  source 1..17: mol_type = other DNA
attatttctcaccctga

SEQ ID NO: 473 - DNA, 17 bp, synthetic construct
  source 1..17: mol_type = other DNA
atcatctcccacccgga

SEQ ID NO: 474 - DNA, 34 bp, synthetic construct
  source 1..34: mol_type = other DNA
aataggtctgagaacgcccattctcagacgtatt

SEQ ID NO: 475 - DNA, 34 bp, synthetic construct
  source 1..34: mol_type = other DNA
gaagttcctatactttctagagaataggaacttc

SEQ ID NO: 476 - DNA, 46 bp, synthetic construct
  source 1..46: mol_type = other DNA
ggccggcttgtcgacgacggcgaactccgtcgtcaggatcatccgg

SEQ ID NO: 477 - DNA, 46 bp, synthetic construct
  source 1..46: mol_type = other DNA
ggccggcttgtcgacgacggcggactccgtcgtcaggatcatccgg

SEQ ID NO: 478 - DNA, 46 bp, synthetic construct
  source 1..46: mol_type = other DNA
ggccggcttgtcgacgacggcgcactccgtcgtcaggatcatccgg

SEQ ID NO: 479 - DNA, 46 bp, synthetic construct
  source 1..46: mol_type = other DNA
ggccggcttgtcgacgacggcgtactccgtcgtcaggatcatccgg

SEQ ID NO: 480 - DNA, 46 bp, synthetic construct
  source 1..46: mol_type = other DNA
ggccggcttgtcgacgacggcgagctccgtcgtcaggatcatccgg

SEQ ID NO: 481 - DNA, 46 bp, synthetic construct
  source 1..46: mol_type = other DNA
ggccggcttgtcgacgacggcgggctccgtcgtcaggatcatccgg

SEQ ID NO: 482 - DNA, 46 bp, synthetic construct
  source 1..46: mol_type = other DNA
ggccggcttgtcgacgacggcgcgctccgtcgtcaggatcatccgg

SEQ ID NO: 483 - DNA, 46 bp, synthetic construct
  source 1..46: mol_type = other DNA
ggccggcttgtcgacgacggcgtgctccgtcgtcaggatcatccgg

SEQ ID NO: 484 - DNA, 46 bp, synthetic construct
  source 1..46: mol_type = other DNA
ggccggcttgtcgacgacggcgacctccgtcgtcaggatcatccgg

SEQ ID NO: 485 - DNA, 46 bp, synthetic construct
  source 1..46: mol_type = other DNA
ggccggcttgtcgacgacggcggcctccgtcgtcaggatcatccgg

SEQ ID NO: 486 - DNA, 46 bp, synthetic construct
  source 1..46: mol_type = other DNA
ggccggcttgtcgacgacggcgccctccgtcgtcaggatcatccgg

SEQ ID NO: 487 - DNA, 46 bp, synthetic construct
  source 1..46: mol_type = other DNA
ggccggcttgtcgacgacggcgtcctccgtcgtcaggatcatccgg

SEQ ID NO: 488 - DNA, 46 bp, synthetic construct
  source 1..46: mol_type = other DNA
ggccggcttgtcgacgacggcgatctccgtcgtcaggatcatccgg

SEQ ID NO: 489 - DNA, 46 bp, synthetic construct
  source 1..46: mol_type = other DNA
ggccggcttgtcgacgacggcgctctccgtcgtcaggatcatccgg

SEQ ID NO: 490 - DNA, 46 bp, synthetic construct
  source 1..46: mol_type = other DNA
ggccggcttgtcgacgacggcgttctccgtcgtcaggatcatccgg

SEQ ID NO: 491 - DNA, 27 bp, synthetic construct
  source 1..27: mol_type = other DNA
tttaccttgattgagatgttaattgtg

SEQ ID NO: 492 - DNA, 50 bp, synthetic construct
  source 1..50: mol_type = other DNA
gcgagtttttatttcgtttatttcaattaaggtaactaaaaaactccttt

SEQ ID NO: 493 - DNA, 34 bp, synthetic construct
  source 1..34: mol_type = other DNA
ctggatcatctggatcactttcgtcaaaaacctg

SEQ ID NO: 494 - DNA, 57 bp, synthetic construct
  source 1..57: mol_type = other DNA
ttcgggtgctgggttgttgtctctggacagtgatccatgggaaactactcagcacca

SEQ ID NO: 495 - DNA, 38 bp, synthetic construct
  source 1..38: mol_type = other DNA
ccccaactggggtaacctttgagttctctcagttgggg

(19) United States
(12) Patent Application Publication
(10) Pub. No.: US 2023/0407280 A1
(43) Pub. Date: Dec. 21, 2023

(54) PROGRAMMABLE GENE EDITING USING GUIDE RNA PAIR

(71) Applicant: Massachusetts Institute of Technology, Cambridge, MA (US)

(72) Inventors: Omar Abudayyeh, Cambridge, MA (US); Jonathan Gootenberg, Cambridge, MA (US)

(21) Appl. No.: 18/303,527

(22) Filed: Apr. 19, 2023

Related U.S. Application Data
(60) Provisional application No. 63/363,310, filed on Apr. 20, 2022.

(51) Int. Cl. C12N 9/22; C12N 15/86; C12N 15/10

(52) U.S. Cl. CPC C12N 9/22; C12N 2750/14143; C12N 15/102; C12N 15/86

(57) ABSTRACT

Provided herein are compositions, methods, and systems comprising a DNA binding nickase, a reverse transcriptase, an integration enzyme, and a guide RNA pair. Also described herein are method of use of the guide RNA pair in methods of editing and integrating polynucleotide sequences.

### CROSS REFERENCE TO RELATED APPLICATION

This application claims the benefit of U.S. Provisional Patent Application Ser. No. 63/363,310, filed Apr. 20, 2022. The entire content of the above-referenced patent application is incorporated by reference in their entirety herein.

### STATEMENT AS TO FEDERALLY FUNDED RESEARCH

This invention was made with government support under EB031957 and AI49694 awarded by the National Institutes of Health. The government has certain rights in the invention.

### SEQUENCE LISTING

The instant application contains a Sequence Listing which has been submitted electronically in XML format and is hereby incorporated by reference in its entirety. Said XML copy, created on Apr. 11, 2023, is named 740487 083474-036 SL.xml and is 494,677 bytes in size.

### BACKGROUND

Editing genomes using the RNA-guided DNA targeting principle of CRISPR (Clustered Regularly Interspaced Short Palindromic Repeats-CRISPR associated proteins) has become a popular in a wide variety of applications. The main advantage of CRISPR system lies in the minimal requirement for programmable DNA interference: an endonuclease, such as a Cas9, Cas12, or any programmable nucleases, which is guided by a customizable RNA structure. Cas9 nuclease is a multi-domain enzyme that uses an HNH nuclease domain to cleave a target nucleic acid strand. The CRISPR/Cas9 protein-RNA complex is directed to and is localized on the target by a guide RNA, then it cleaves the target to generate a DNA double strand break (dsDNA break, DSB). After cleavage, DNA repair mechanisms are activated to repair the cleaved strand. Repair mechanisms are generally two types: non-homologous end joining (NHEJ) or homologous recombination (HR). Basically, NHEJ dominates repair, and, being error prone, generates random indels (insertions or deletions) causing frame shift mutations, among others. In contrast, HR has a more precise repairing capability and is potentially capable of incorporating the exact substitution or insertion. To enhance HR, several techniques have been tried, for example: combination of fusion proteins of Cas9 nuclease with homology-directed repair (HDR) effectors to enforce their localization at DSBs, introducing an overlapping homology arm, or suppression of NHEJ. Most of these techniques rely on the host DNA repair systems.

Recently, a new genetic editing system for site-specific genetic engineering using Programmable Addition via Site-Specific Targeting Elements (PASTE) has been developed (See, e.g., loannidi et al., “Drag-and-drop genome insertion without DNA cleavage with CRISPRdirected integrases,” bioRxiv preprint, 2021, doi: https://doi.org/10.1101/2021.1101 466786; and U.S. patent application Ser. No. 17/451,734, the entire contents of each are hereby incorporated by reference in their entirety). PASTE comprises the addition of an integration site into the target genome followed by the insertion of one or more genes of interest or one or more nucleic acid sequences of interest at the site. PASTE combines gene editing technologies and integrase technologies to achieve unidirectional incorporation of genes in a genome for the treatment of diseases and diagnosis of disease. Despite these developments, the insertion of long sequences into the target genome is still a challenge.

Therefore, there is a need for more effective tools for gene editing and delivery.

### SUMMARY

The present disclosure provides compositions and systems for programmable gene editing that utilize, comprising a DNA binding nickase, a reverse transcriptase, an integration enzyme, and a guide RNA pair comprising heterologous gRNAs each separately comprising a scaffold sequence, a primer binding sequence, an integration sequence, a spacer sequence, and optionally a reverse transcription template sequence. In one aspect, provided herein is a composition comprising: a DNA binding nickase or a functional fragment or variant thereof; a reverse transcriptase (RT) or a functional fragment or variant thereof; an integration enzyme or a functional fragment or variant thereof, wherein the integration enzyme is selected from the group consisting of an integrase, a recombinase, and a reverse transcriptase; and a guide RNA (gRNA) pair comprising: a first heterologous gRNA or functional fragments or variants thereof, comprising: a first spacer sequence, a first scaffold sequence, a first reverse transcription template sequence that comprises at least a first portion of an at least first integration recognition sequence; a first primer binding sequence, and a second heterologous gRNA or functional fragment or variant thereof, comprising: a second spacer sequence, a second scaffold sequence, a second reverse transcription template sequence that comprises at least a second portion of the first integration recognition sequence, a second primer binding sequence, wherein the first heterologous RNA and the second heterologous RNA collectively encode the entirety of the first integration recognition sequence.

In some embodiments, the first primer binding sequence, the second primer binding sequence, or both, are at least about 9 nucleotides in length or about 9-15 nucleotides in length.

In some embodiments, the at least first integration recognition sequence is at least about 38 nucleotides in length or about 38-46 nucleotides in length.

In some embodiments, the first heterologous gRNA does not comprise a reverse transcription template sequence or the first and second heterologous gRNAs do not comprise a reverse transcription template sequence.

In some embodiments, the first reverse transcription template sequence, the second reverse transcription template sequence, or both, are about 1-34 nucleotides in length.

In some embodiments, the first spacer sequence, the second spacer sequence, or both, are at least about 20 nucleotides in length or about 17-21 nucleotides in length.

In some embodiments, the first scaffold sequence, the second scaffold sequence, or both, are at least about 60 nucleotides in length or about 60-120 nucleotides in length.

In some embodiments, the first reverse transcription template sequence encodes a first extended sequence, and the second reverse transcription template sequence encodes a second extended sequence.

In some embodiments, the first and second extended sequences comprise at least about 5 complementary nucleotides with respect to each other, about 5-10 complementary nucleotides with respect to each other, about 11-20 complementary nucleotides with respect to each other, or about 21-30 complementary nucleotides with respect to each other, about 31-40 complementary nucleotides with respect to each other, about 41-50 complementary nucleotides with respect to each other, or about 51-60 complementary nucleotides with respect to each other.

In some embodiments, annealing of the complementary nucleotides forms a duplex which results in an insertion of the at least first integration recognition sequence into a target location.

In some embodiments, the first and second heterologous gRNAs form a double stranded nucleic acid.

In some embodiments, the first spacer sequences and the second space sequence are separated by at least about 0-1000 nucleotides in the genome.

In some embodiments, the first and second heterologous gRNAs comprise from 5′-3′ in this order the spacer sequence, the scaffold sequence, the integration sequence, and the primer binding sequence.

In some embodiments, the DNA binding nickase is a Cas9-D10A, a Cas9-H840A, a Cas12a nickase, or a Cas12b nickase, or a functional fragment or variant thereof

In some embodiments, the reverse transcriptase is derived from Moloney Murine Leukemia Virus (M-MLV) reverse transcriptase, transcription xenopolymerase (RTX), avian myeloblastosis virus reverse transcriptase (AMV-RT), or Eubacterium rectale maturase RT (MarathonRT).

In some embodiments, the reverse transcriptase comprises a mutation relative to the wild-type sequence. In some embodiments, the reverse transcriptase is a M-MLV reverse transcriptase, an AMV-RT, MarathonRT, or a RTX, optionally the reverse transcriptase is a modified M-MLV reverse transcriptase relative to the wildtype M-MLV reverse transcriptase, and optionally the M-MLV reverse transcriptase domain comprises one or more of the mutations selected from the group consisting of D200N, T306K, W313F, T330P, and L603W.

In some embodiments, the first scaffold sequence, the second scaffold sequence, or both, comprises at least 80% sequence identity to any of the nucleic acid sequences set forth in Table A.

In some embodiments, the integration recognition sequence comprises at least 80% sequence identity to any one of the nucleic acid sequences set forth in Table B.

In some embodiments, the first and second heterologous gRNAs comprise the nucleic acid sequence of SEQ ID NO: 1-80, SEQ ID NO: 81-160, SEQ ID NO: 161-362, SEQ ID NO: 363-372, or SEQ ID NO: 373-394.

In some embodiments, the integration enzyme is Dre, Vika, Bxb1, φC31, RDF, FLP, φBT1, R1, R2, R3, R4, R5, TP901-1, A118, φFC1, φC1, MR11, TG1, φ370.1, WO, BL3, SPBc, K38, Peaches, Veracruz, Rebeuca, Theia, Benedict, KSSJEB, PattyP, Doom, Scowl, Lockley, Switzer, Bob3, Troube, Abrogate, Anglerfish, Sarfire, SkiPole, ConceptII, Museum, Severus, Airmid, Benedict, Hinder, ICleared, Sheen, Mundrea, BxZ2, (pRV, retrotransposases encoded by R2, L1, Tol2 Tc1, Tc3, Mariner (Himar 1), Mariner (mos 1), or Minos, or any functional fragments or variants thereof

In some embodiments, the integration enzyme is Bxb1 or any functional fragments or variants thereof.

In some embodiments, the integration sequence is an attB sequence, an attP sequence, an attL sequence, an attR sequence, a Vox sequence, a FRT sequence, or a functional fragment or variant thereof

In some embodiments, the integration sequence is an attB sequence, optionally the attB sequence comprises about 38-46 base pairs.

In some embodiments, the integration sequence is an attp sequence, optionally the attp sequence comprises about 48-52 base pairs.

In some embodiments, the DNA binding nickase is a Cas9-D10A, a Cas9-H840A, a Cas12a/b/c/d/e/f/h/i/j, or a functional fragment or variant thereof

In another aspect, provided herein is a method of site-specifically integrating an exogenous nucleic acid into a cell genome, the method comprising: (a) incorporating an integration sequence at a target location in the cell genome by introducing into a cell: (i) a DNA binding nickase or a functional fragment or variant thereof; (ii) a reverse transcriptase (RT) or a functional fragment or variant thereof; and (iii) a guide RNA (gRNA) pair comprising a first heterologous gRNA or functional fragments or variants thereof, comprising: a first spacer sequence, a first scaffold sequence, a first reverse transcription template sequence that comprises at least a first portion of an at least first integration recognition sequence; a first primer binding sequence and a second heterologous gRNA or functional fragments or variants thereof, comprising: a second spacer sequence, a second scaffold sequence, a second reverse transcription template sequence that comprises at least a second portion of the first integration recognition sequence, a second primer binding sequence , wherein: the first and second heterologous gRNAs interact with the DNA binding nickase and target the target location in the cell genome, the DNA binding nickase nicks a strand of the cell genome, and the reverse transcriptase reverse transcribes (i) the first reverse transcription template sequence into a first extended sequence that encodes the at least first portion of the first integration recognition sequence and (ii) the second reverse transcription template sequence into a second extended sequence that encodes the at least second portion of the first integration recognition sequence, the first and second extended sequences comprise at least about 5 complementary nucleotides with respect to each other, wherein annealing of the complementary nucleotides forms a duplex which results in an insertion of the at least first integration recognition sequence into the target location. The method further comprises: (b) integrating the nucleic acid into the cell genome by introducing into the cell: (i) a DNA or RNA strand comprising the nucleic acid linked to a sequence that is complementary or associated to the integration sequence; and (ii) an integration enzyme or a functional fragment or variant thereof, wherein the integration enzyme is selected from the group consisting of an integrase, a recombinase, and a reverse transcriptase, wherein the integration enzyme incorporates the nucleic acid into the cell genome at the at least first integration recognition sequence by integration, recombination, or reverse transcription of the sequence that is complementary or associated to the integration sequence, thereby introducing the nucleic acid into the target location of the cell genome of the cell.

In some embodiments, the first and second heterologous gRNAs hybridize to a complementary strand of the cell genome to the genomic strand that is nicked by the DNA binding nickase, optionally the integration enzyme is introduced as a peptide or a nucleic acid encoding the integration enzyme, optionally DNA binding nickase is introduced as a peptide or a nucleic acid encoding the DNA binding nickase, optionally the DNA or RNA strand comprising the nucleic acid is introduced into the cell as a minicircle, a plasmid, mRNA or a linear DNA, optionally the DNA or RNA strand comprising the nucleic acid is between 1000 bp and 36,000 bp, optionally the DNA or RNA strand comprising the nucleic acid is more than 36,000 bp, optionally the DNA or RNA strand comprising the nucleic acid is less than 1000 bp, and optionally the DNA comprising the nucleic acid is introduced into the cell as a minicircle.

In some embodiments, the minicircle does not comprise a sequence of a bacterial origin.

In some embodiments, the DNA binding nickase is linked to the reverse transcriptase, and the DNA binding nickase linked to the reverse transcriptase domain and the integration enzyme are linked via a linker.

In some embodiments, the linker is cleavable,

In some embodiments, the linker is non-cleavable.

In some embodiments, the linker can be replaced by two associating binding domains of the DNA binding nickase linked to the reverse transcriptase.

In some embodiments, the DNA binding nickase, the reverse transcriptase, the gRNA pair, the DNA or RNA comprising nucleic acid linked to a complementary or associated integration sequence, and the integration enzyme are introduced into a cell in a single reaction.

In some embodiments, the nucleic acid is introduced into the cell as an adeno-associated virus (AAV) or an adenovirus (AdV).

In some embodiments, the DNA binding nickase, the reverse transcriptase, the gRNA pair, the DNA or RNA comprising nucleic acid linked to a complementary or associated integration sequence, and the integration enzyme are introduced using a virus, a RNP, an mRNA, a lipid, or a polymeric nanoparticle.

In some embodiments, the nucleic acid is a reporter gene, and optionally the reporter gene is a fluorescent protein.

In some embodiments, the cell is a dividing cell.

In some embodiments, the cell is a non-dividing cell.

In some embodiments, the target location in the cell genome is the locus of a mutated gene.

In some embodiments, the nucleic acid is a degradation tag for programmable knockdown of proteins in the presence of small molecules.

In some embodiments, the cell is a mammalian cell, a bacterial cell, or a plant cell.

In some embodiments, the nucleic acid is a T-cell receptor (TCR), a chimeric antigen receptor (CAR), an interleukin, a cytokine, or an immune checkpoint gene for integration into a T-cell or natural killer (NK) cell, and optionally the TCR, the CAR, the interleukin, the cytokine, or the immune checkpoint gene is incorporated into the target site of the T-cell or NK cell genome using a minicircle DNA.

In some embodiments, the nucleic acid is a beta hemoglobin (HBB) gene and the cell is a hematopoietic stem cell (HSC), optionally the HBB gene is incorporated into the target site in the HSC genome using a minicircle DNA, and optionally the nucleic acid is a gene responsible for beta thalassemia or sickle cell anemia.

In some embodiments, the nucleic acid is a metabolic gene, optionally metabolic gene is involved in alpha-1 antitrypsin deficiency or ornithine transcarbamylase (OTC) deficiency, and optionally the metabolic gene is a gene involved in an inherited disease.

In some embodiments, the nucleic acid is a gene involved in an inherited disease or an inherited syndrome, and optionally the inherited disease is cystic fibrosis, familial hypercholesterolemia, adenosine deaminase (ADA) deficiency, X-linked SCID (X-SCID), Wiskott-Aldrich syndrome (WAS), hemochromatosis, Tay-Sachs, fragile X syndrome, Huntington's disease, Marfan syndrome, phenylketonuria, or muscular dystrophy.

In another aspect, provided herein is a nucleic acid molecule encoding the DNA binding nickase, the reverse transcriptase, the integration enzyme, and the gRNA pair. In another aspect, provided herein is a vector comprising the nucleic acid molecule.

In another aspect, provided herein is a cell comprising the composition, the nucleic acid molecule, or the vector.

In some embodiments, the cell is a prokaryotic cell.

In some embodiments, the cell is a eukaryotic cell.

In some embodiments, the eukaryotic cell is a mammalian cell, and optinally the mammalian cell is a human cell.

In another aspect, provided herein is a gRNA pair that specifically binds to a DNA binding nickase, wherein the gRNA pair comprises a first heterologous gRNA or functional fragments or variants thereof, and a second heterologous gRNA or functional fragments or variants thereof, and wherein the first and second heterologous gRNAs separately comprise a scaffold sequence, a primer binding sequence, an integration sequence, a spacer sequence, and optionally a reverse transcription template sequence.

In another aspect, provided herein is a polypeptide comprising a DNA binding nuclease comprising a nickase activity C-terminally linked to a reverse transcriptase linked to an integration enzyme via a linker.

In some embodiment: the linker is cleavable or non-cleavable; the integration enzyme is fused to an estrogen receptor; the DNA binding nuclease comprising a nickase activity is selected from the group consisting of Cas9-D10A, Cas9-H840A, and Cas12a/b/c/d/e/f/g/h/i/j; the reverse transcriptase is a M-MLV reverse transcriptase, a AMV-RT, a MarathonRT, or a XRT, optionally wherein the reverse transcriptase is a modified M-MLV relative to a wild-type M-MLV reverse transcriptase, optionally wherein the M-MLV reverse transcriptase domain comprises one or more of mutations selected from the group consisting of D200N, T306K, W313F, T330P, and L603W; the integration enzyme is selected from group consisting of Cre, Dre, Vika, Bxb1, φC31, RDF, FLP, φBT1, R1, R2, R3, R4, R5, TP901-1, A118, φFC1, φC1, MR11, TG1, φ370.1, WO, BL3, SPBc, K38, Peaches, Veracruz, Rebeuca, Theia, KSSJEB, PattyP, Doom, Scowl, Lockley, Switzer, Bob3, Troube, Abrogate, Anglerfish, Sarfire, SkiPole, Conceptll, Museum, Severus, Airmid, Benedict, Hinder, ICleared, Sheen, Mundrea, BxZ2, φRV, retrotransposases encoded by R2, L1, To12 Tc1, Tc3, Mariner (Himar 1), Mariner (mos 1), Minos, and any mutants thereof.

### BRIEF DESCRIPTION OF THE DRAWINGS

FIG. 1A is a schematic diagram showing PASTE elements such as a Cas9-RT, a pegRNA containing the integrase attachment site (i.e., atgRNA), a nicking guide, and an integrase. The Cas9-RT combined with the nicking guide and pegRNA containing the atgRNA inserts an integration sequence which serves as a “beacon” for a cognate integrase.

FIG. 1B is a schematic diagram showing the recombination of attP and attB sites when in presence of a serine integrase. For integration of DNA, attP and attB sites must be in the same orientation.

FIG. 1C is a schematic diagram showing atgRNA parameters such as a Cas9 spacer sequence which targets a relevant locus, a primer binding site (PBS) which binds a single stranded DNA R-Loop generated by Cas9 and allows for priming of a reverse transcriptase, an integrase insertion site sequence containing the attB landing site, an overlap region with a genome (reverse transciption template, RT), and relative locations and efficacy of the atgRNA spacer and nicking guide.

FIG. 2 is a schematic diagram showing the cleavage of a double stranded nucleotide using two heterologous atgRNAs (i.e., paired guides). Sequences (shown in red lines) are growing attachment sites with the aid of paired guides. The paired guides are partially complementary to each other and allow a double stranded intermediate promoting higher integration rates of the integrase attachment site versus a competing DNA repair to correct the “genome flaps” wild-type sequence.

FIG. 3 is a bar graph showing the attB percent integration at the ACTB locus in a HEK293FT cell line using a panel of 40 different paired guides corresponding to SEQ ID NOs: 1-80 (labels: “paired combo 1-40”) relative to controls (labels: “pDY0207” is a single atgRNA, “pDY0209” is a nicking guide, and “pDY077” is an empty control vector).

FIG. 4 is a bar diagram showing the attB percent integration at the DNMT1 mouse locus in a Hepal-6 cell line using a panel of 40 paired guides corresponding to SEQ ID NOs: 81-160 (labels: “paired combo 1-40”) relative to controls (labels: “pDY1055 DMNT1 guide 2” is a single atgRNA plus a nicking guide).

FIG. 5 is a bar graphs showing the attB percent integration at the mouse NOLC1 locus in a Hepa 1-6 cell line using a panel of 6 paired guides corresponding to SEQ ID NOs: X-Z (labels: “paired aRY1039 B6”, “paired aRY1039 B7”, “paired aRY1039 B6”, “paired aRY1039 paired A5”, “paired aRY1039 B7”, and “paired pDY1192”) relative to controls encompassing 49 distinct combinations of single atgRNA guide plus a nicking guide (partial labels: “original combo”).

FIG. 6 is a bar graphs showing the eGFP percent integration at the human NOLC1 locus in a HEK293FT cell line after using 4 distinct paired guides for the attB site corresponding to SEQ ID NOs: 363-370 (labels: “PASTE replace pair 1-4” relative to controls which include a single atgRNA guide plus a nicking guide labeled “PASTEv3” corresponding to SEQ ID NOs: 371-372 and a no PRIME control.

FIG. 7 is a bar graphs showing the eGFP percent integration at the mouse NOLC1 locus in a Hepa-1-6 cell line after using 11 distinct combinations of paired guides for the attB site corresponding to SEQ ID NOs: 373-394 (labels: “aRY1039 B6+aRY1039 A1”, “aRY1039 B7+aRY1039 A9”, “aRY1039 B1+aRY1039 B4”, “aRY1039Al2+aRY1039 B2”, “aRY1039 B6+aRY1039 A2”, “aRY1039 A4+aRY1039 A6”, “aRY1039 B7+aRY1039 A6”, “aRY1039 A12+aRY1039 B4”, “aRY1039 B1+aRY1039 B2”, “aRY1039 B1+aRY1039B3”) relative to controls.

FIG. 8 is a bar graphs showing the eGFP percent integration into the attB site using SpCas9-RT-P2A-Blast Bxb1 and paired guides at the mouse NOLC locus in a Hepa 1-6 cell line using a paired guide (labels: “mouse NOLC1 region forward pair with rev 38 bp AttB guide 7+2” or “mouse NOLC1 region forward pair with rev 38bp AttB guide 5”). SpCas9-RT-P2A-Blast Bxb1, paired guides, and eGFP were transfected. Cargo containing eGFP delivered to a Hepa-1-6- cell line via two distinct AdV delivery vector cocktails labeled, “viraquest” and “vector biolabs,” respectively in a limited dilution series.

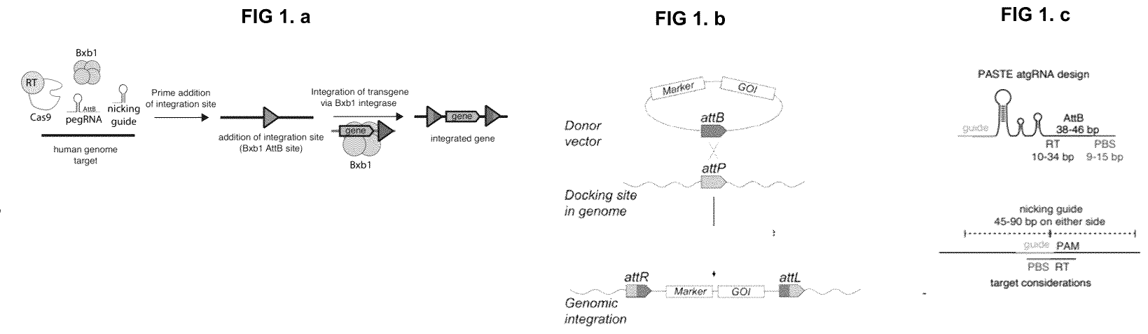

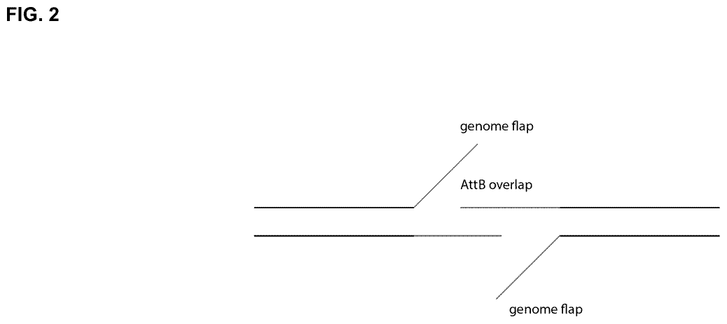

### DETAILED DESCRIPTION

PASTE editing utilizes a modified PRIME gene editing technique to site-specifically insert an integration site within a target polynucleotide (e.g., genome) and subsequently utilizing the site to integrate a polynucleotide of interest (See, e.g., US20220145293, the entire contents of which are incorporated by reference herein for all purposes). PASTE-REPLACE editing utilizes PASTE but with a paired set of gRNAs that enable the simultaneous deletion of a polynucleotide sequence (e.g., a gene) and replacement of the polynucleotide with an exogenous polynucleotide of interest (e.g., a variant gene). The first step in PASTE and PASTE-REPLACE editing generally comprises the use of a nickase (e.g., a Cas9 nickase) fused to a reverse transcriptase and an extended gRNA (pegRNA). The pegRNA comprises at least three functional polynucleotides (i) a targeting sequence (targeting the nickase to the target polynucleotide site), (ii) a primer binding site (PBS), and (iii) a reverse transcriptase template sequence containing the integration site. However, providing all three of these functionalities in a single RNA molecule means the pegRNAs are relatively long (typically 150-200 nucleotides) making the pegRNA difficult and expensive to manufacture at a large scale, as would be required for therapeutic or diagnostic uses. Additionally, the long length of the pegRNAs may impact editing efficiency; for example, biochemical measurements show that the complex design of the pegRNA reduces its affinity to Cas9, and likely decreases the efficiency of the process. As such, the current disclosure provides improved PASTE editing systems that allow for efficient editing and enhanced manufacturability. Providing a gRNA pair was found to be particularly advantageous in technologies like PASTE because it allows the insertion of long (38-46 bp) integration sites (versus PRIME editing which in many instances requires only short reverse transcriptase template sequences encoding a single nucleotide change).

### 7.1. Definitions

The section headings used herein are for organizational purposes only and are not to be construed as limiting the subject matter described.

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed.

The use of the singular forms herein includes the plural unless specifically stated otherwise. As used herein, the singular forms “a,” “an,” and “the” include plural referents unless the context clearly dictates otherwise. Furthermore, use of the term “including” as well as other forms, such as “include,” “includes,” and “included,” is not limiting.

It is understood that wherever aspects are described herein with the language “comprising,” otherwise analogous aspects described in terms of “consisting of” and/or “consisting essentially of” are also provided.

Units, prefixes, and symbols are denoted in their Systeme International de Unites (SI) accepted form. Numeric ranges are inclusive of the numbers defining the range.

As described herein, any concentration range, percentage range, ratio range or integer range is to be understood to include the value of any integer within the recited range and, when appropriate, fractions thereof (such as one tenth and one hundredth of an integer), unless otherwise indicated.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. When particular values or compositions are provided in the application and claims, unless otherwise stated, the meaning of “about” or “comprising essentially of” should be assumed to be within an acceptable error range for that particular value or composition.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

When proteins are contemplated herein, it should be understood that polynucleotides encoding the proteins are also provided, as are vectors comprising the polynucleotides encoding the proteins.

As used herein, the term “Cas9” refers to an RNA-guided nuclease comprising a Cas9 domain, or a functional fragment or variant thereof (e.g., a protein comprising an active or inactive DNA cleavage domain of Cas9, and/or the gRNA binding domain of Cas9).

As used herein, the term “DNA binding nickase” such as a Cas9 or Cas12 nickase refers to a variant of DNA binding nuclease which is capable of cleaving only one strand of a target double stranded polynucleotide, thereby introducing a single-strand break in the target double strand polynucleotide. Similar terminology is used herein in reference to other Cas nucleases that exhibit nickase activity. For example, a “Cas12e nickase” would be used similarly herein to refer to a Cas12e which is capable of cleaving only one strand of a target double stranded polynucleotide, thereby introducing a single-strand break in the target double strand polynucleotide

As used herein, the term “derived from,” with reference to a polynucleotide sequence refers to a polynucleotide sequence that has at least 85%, 90%, 95%, 96%, 97%, 98%, or 99% sequence identity to a reference naturally occurring nucleic acid sequence from which it is derived. The term “derived from,” with reference to an amino acid sequence refers to an amino acid sequence that has at least 85%, 90%, 95%, 96%, 97%, 98%, or 99% sequence identity to a reference naturally occurring amino acid sequence from which it is derived. The term “derived from” as used herein does not denote any specific process or method for obtaining the polynucleotide or amino acid sequence. For example, the polynucleotide or amino acid sequence can be chemically synthesized.

As used herein, the term “DNA” or “DNA polynucleotides” refers to macromolecules that include multiple deoxyribonucleotides that are polymerized via phosphodiester bonds. Deoxyribonucleotides are nucleotides in which the sugar is deoxyribose.

As used herein, the term “functional fragment” in reference to a nucleic acid sequence, an amino acid sequence, or the like refers to a fragment of a reference nucleic acid sequence, an amino acid sequence, or the like that retains at least one particular function. For example, a functional fragment of an aptamer binding protein can refer to a fragment of the protein that retains the ability to bind the cognate aptamer. Not all functions of the reference protein need be retained by a functional fragment of the protein. In some instances, one or more functions are selectively reduced or eliminated.

As used herein, the term “functional variant” in reference to a nucleic acid sequence, an amino acid sequence, or the like refers to a nucleic acid sequence, an amino acid sequence, or the like that comprises at least one nucleic acid or amino acid modification (e.g., a substitution, deletion, addition) compared to the nucleic acid or amino acid sequence of a reference nucleic acid sequence, an amino acid sequence, or the like, that retains at least one particular function. For example, a functional variant of an aptamer binding protein refers to a protein that binds an aptamer comprising an amino acid substitution as compared to a wild type reference protein that retains the ability to bind the cognate aptamer. Not all functions of the reference wild type protein need be retained by the functional variant of the protein. In some instances, one or more functions are selectively reduced or eliminated.

As used herein, the term “fusion protein” and grammatical equivalents thereof refer to a protein that comprises an amino acid sequence derived from at least two separate proteins. The amino acid sequence of the at least two separate proteins can be directly connected through a peptide bond; or can be operably connected through an amino acid linker. Therefore, the term fusion protein encompasses embodiments, wherein the amino acid sequence of e.g., Protein A is directly connected to the amino acid sequence of Protein B through a peptide bond (Protein A-Protein B), and embodiments, wherein the amino acid sequence of e.g., Protein A is operably connected to the amino acid sequence of Protein B through an amino acid linker (Protein A-linker-Protein B).

A used herein, the term “fuse” and grammatical equivalents thereof refer to the operable connection of an amino acid sequence derived from one protein to the amino acid sequence derived from different protein. The term fuse encompasses both a direct connection of the two amino acid sequences through a peptide bond, and the indirect connection through an amino acid linker.

As used herein, the term “guide RNA” or “gRNA” refers to an RNA polynucleotide that guides the insertion or deletion of one or more polynucleotides of interest (e.g., a gene of interest) into a target polynucleotide (e.g., genome) via a nuclease, nickase, or functional fraction or variant thereof (e.g., a Cas protein, e.g., Cas9).

As used herein, the term “integrase” refers to a protein capable of integrating a polynucleotide of interest (e.g., a gene) into a desired location or target site (e.g., at an integration site) in a target polynucleotide (e.g., the genome of a cell). The integration can occur in a single reaction or multiple reactions.

As used herein, the term “integration sequence” refers to a polynucleotide sequence that encodes an integration site.

As used herein, the term “integration site” refers to a polynucleotide sequence capable of being recognized by an integrase.

As used herein, the term “modification,” with reference to a polynucleotide sequence, refers to a polynucleotide sequence that comprises at least one substitution, alteration, inversion, addition, or deletion of nucleotide compared to a reference polynucleotide sequence. Modifications can include the inclusion of non-naturally occurring nucleotide residues. As used herein, the term “modification,” with reference to an amino acid sequence refers to an amino acid sequence that comprises at least one substitution, alteration, inversion, addition, or deletion of an amino acid residue compared to a reference amino acid sequence. Modifications can include the inclusion of non-naturally occurring amino acid residues. Naturally occurring amino acid derivatives are not considered modified amino acids for purposes of determining percent identity of two amino acid sequences. For example, a naturally occurring modification of a glutamate amino acid residue to a pyroglutamate amino acid residue would not be considered an amino acid modification for purposes of determining percent identity of two amino acid sequences. Further, for example, a naturally occurring modification of a glutamate amino acid residue to a pyroglutamate amino acid residue would not be considered an amino acid “modification” as defined herein.

As used herein, the term “nickase” refers to a protein (e.g., a nuclease) that has the ability to cleave only one strand of a target double stranded polynucleotide, thereby introducing a single-strand break in the target double strand polynucleotide. In some embodiments, for example, an editing polypeptide described herein comprises a Cas9 nuclease with one of the two nuclease domains inactivated, e.g., by amino acid substitution of H840A, wherein the Cas9 has nickase activity but is not able to make a double strand break in a target double stranded polynucleotide.

As used herein, the terms “operably connected” and “operably linked” are used interchangeably and refer to a linkage of polynucleotide sequence elements or polypeptide sequence elements in a functional relationship. For example, a polynucleotide sequence is operably connected when it is placed into a functional relationship with another polynucleotide sequence. In some embodiments, a transcription regulatory polynucleotide sequence e.g., a promoter, enhancer, or other expression control element is operably-linked to a polynucleotide sequence that encodes a protein if it affects the transcription of the polynucleotide sequence that encodes the protein.

As used herein, the term “orthogonal integration sites” refers to integrations sites that do not significantly recognize the recognition site or nucleotide sequence of the integrase (e.g., recombinase) recognized by the other.

The determination of “percent identity” between two sequences (e.g., polypeptide or polynucleotides) can be accomplished using a mathematical algorithm. A specific, non-limiting example of a mathematical algorithm utilized for the comparison of two sequences is the algorithm of Karlin S & Altschul S F (1990) PNAS 87: 2264-2268, modified as in Karlin S & Altschul SF (1993) PNAS 90: 5873-5877, each of which is herein incorporated by reference in its entirety. Such an algorithm is incorporated into the NBLAST and XBLAST programs of Altschul SF et al., (1990) J Mol Biol 215: 403, which is herein incorporated by reference in its entirety. BLAST nucleotide searches can be performed with the NBLAST nucleotide program parameters set, e.g., for score=100, wordlength=12 to obtain nucleotide sequences homologous to a nucleic acid molecule described herein. BLAST protein searches can be performed with the XBLAST program parameters set, e.g., to score 50, wordlength=3 to obtain amino acid sequences homologous to a protein molecule described herein. To obtain gapped alignments for comparison purposes, Gapped BLAST can be utilized as described in Altschul SF et al., (1997) Nuc Acids Res 25: 3389-3402, which is herein incorporated by reference in its entirety. Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (See, e.g., National Center for Biotechnology Information (NCBI) on the worldwide web, ncbi.nlm.nih.gov). Another specific, non-limiting example of a mathematical algorithm utilized for the comparison of sequences is the algorithm of Myers and Miller, 1988, CABIOS 4:11-17, which is herein incorporated by reference in its entirety. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

As used herein the term “pharmaceutical composition” means a composition that is suitable for administration to an animal, e.g., a human subject, and comprises a therapeutic agent and a pharmaceutically acceptable carrier or diluent. A “pharmaceutically acceptable carrier or diluent” means a substance for use in contact with the tissues of human beings and/or non-human animals without excessive toxicity, irritation, allergic response, or other problem or complication, commensurate with a reasonable therapeutic benefit/risk ratio.

The terms “polynucleotide,” “nucleic acid,” and “nucleic acid molecule” are used interchangeably herein and refer to a polymer of DNA or RNA. The nucleic acid molecule can be single-stranded or double-stranded; contain natural, non-natural, or altered nucleotides; and contain a natural, non-natural, or altered internucleotide linkage, such as a phosphoroamidate linkage or a phosphorothioate linkage, instead of the phosphodiester found between the nucleotides of an unmodified nucleic acid molecule. Nucleic acid molecules include, but are not limited to, all nucleic acid molecules which are obtained by any means available in the art, including, without limitation, recombinant means, e.g., the cloning of nucleic acid molecules from a recombinant library or a cell genome, using ordinary cloning technology and polymerase chain reaction, and the like, and by synthetic means. The skilled artisan will appreciate that, except where otherwise noted, nucleic acid sequences set forth in the instant application will recite thymidine (T) in a representative DNA sequence but where the sequence represents RNA (e.g., mRNA), the thymidines (Ts) would be substituted for uracils (Us). Thus, any of the RNA polynucleotides encoded by a DNA identified by a particular sequence identification number may also comprise the corresponding RNA (e.g., mRNA) sequence encoded by the DNA, where each thymidine (T) of the DNA sequence is substituted with uracil (U).

As used herein, the term “polynucleotide of interest” refers to a polynucleotide intended or desired to be integrated into a target polynucleotide using any suitable method (e.g., a method described herein).

As used herein, the term “primer binding site” or “PBS” refers to the portion of a gRNA that binds to the polynucleotides sequence at the 3′ end of the flap that is formed after the DNA binding nickase nicks the target polynucleotide sequence.

The terms “protein” and “polypeptide” are used interchangeably herein and refer to a polymer of at least two amino acids linked by a peptide bond.

As used herein, the term “protospacer” refers to the DNA sequence that has the same (or similar) nucleotide sequence as the spacer sequence of a gRNA. The gRNA anneals to the complement of the protospacer sequence on the opposite strand of the DNA.

As used herein, the term “protospacer adjacent motif” or “PAM” refers to a short DNA sequence, typically 2-6 base pairs, that functions to aid a Cas nickase in recognizing the target DNA.

As used herein, the term “recognition site” refers to a polynucleotide sequence that pairs with an integration site to mediate integration by an integrase (e.g., a recombinase).

As used herein, the term “RNA” or “RNA polynucleotide” refers to macromolecules that include multiple ribonucleotides that are polymerized via phosphodiester bonds. Ribonucleotides are nucleotides in which the sugar is ribose. RNA may contain modified nucleotides; and contain natural, non-natural, or altered internucleotide linkages, such as a phosphoroamidate linkage or a phosphorothioate linkage, instead of the phosphodiester found between the nucleotides of an unmodified nucleic acid molecule.

As used herein, the term “hairpin loop” in reference to an RNA polynucleotide (e.g., an aptamer) refers to an RNA sequence that under physiological conditions is able to base-pair to form a double helix that ends in an unpaired loop.

As used herein, the term “reverse transcriptase” refers to a protein (e.g., a polymerase) that is capable of RNA-dependent DNA synthesis. All known reverse transcriptases require a primer to synthesize a DNA transcript from an RNA template. An exemplary reverse transcriptase commonly used in the art is derived from the moloney murine leukemia virus (M-MLV). See, e.g., Gerard, G. R., DNA 5:271-279 (1986) and Kotewicz, M. L., et al., Gene 35:249-258 (1985).

As used herein, the term “reverse transcriptase template sequence” refers to the portion of a gRNA that encodes the polynucleotide desired to be integrated into the target polynucleotide (e.g., genome) that is synthesized by the reverse transcriptase. The reverse transcriptase template sequence is used as a template during DNA synthesis by the reverse transcriptase.

As used herein, the term “scaffold” in reference to a gRNA refers to a polynucleotide in a gRNA that mediates binding to a nuclease (e.g., nickase) or a functional fragment or variant thereof (e.g., Cas9 (e.g., Cas9 nickases)).

As used herein, the term “spacer” in reference to a gRNA refers to a polynucleotide in a gRNA that mediates binding to a polynucleotide comprising a sequence complementary to the protospacer.

As used herein, the term “therapeutic nucleotide modification” refers to a polynucleotide of interest that encodes at least one nucleotide modification (e.g., substitution, deletion, or insertion) relative to the endogenous target polynucleotide (e.g., gene) sequence that is intended to have or does have a therapeutic effect in a subject.

A “therapeutically effective amount” of a therapeutic agent (e.g., a composition or system described herein) refers to any amount of the therapeutic agent that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

As used herein, the terms “treat,” treating,” “treatment,” and the like refer to reducing or ameliorating a disease and/or symptom(s) associated therewith or obtaining a desired pharmacologic and/or physiologic effect. It will be appreciated that, although not precluded, treating a disease does not require that the disease, or symptom(s) associated therewith be completely eliminated. In some embodiments, the effect is therapeutic, i.e., without limitation, the effect partially or completely reduces, diminishes, abrogates, abates, alleviates, decreases the intensity of, or cures a disease and/or adverse symptom attributable to the disease. In some embodiments, the effect is preventative, i.e., the effect protects or prevents an occurrence or reoccurrence of a disease. To this end, the presently disclosed methods comprise administering a therapeutically effective amount of a compositions as described herein.

### 7.2. PRIME and PASTE

PRIME editing generally involves the use of Cas9 nickase fused to a reverse-transcriptase and an extended gRNA (pegRNA). The pegRNA comprises a standard guide sequence (e.g., a spacer and a scaffold to target the Cas9 to the target site), a PBS) and a reverse transcriptase template sequence containing the desired nucleotide edit (see, e.g., Scholefield, J., Harrison, P. T. Prime editing — an update on the field. Gene Ther 28, 396-401 (2021). https://doi.org/10.1038/s41434-021-00263-9).

In some embodiments, the compositions and systems described herein are useful in the method of PASTE editing. PASTE editing utilizes a modified PRIME technique to site-specifically insert an integration site within a target polynucleotide and subsequently utilizing the site to integrate a polynucleotide sequence of interest (see, e.g., U.S. Ser. No. 17/451,734, the entire contents of which are incorporated by reference herein for all purposes).

### 7.3. DNA Binding Nickases

In some embodiments, the compositions, systems, and methods described herein utilize a DNA binding nickase (or a functional fragment or variant thereof). In some embodiments, a functional fragment or functional variants of a DNA binding nickase is used, wherein the fragment or variant maintains nickase activity.

In some embodiments, the DNA binding nickase is a naturally occurring nickase (or functional fragment or variant thereof). In some embodiments, the DNA binding nickase (or a functional fragment or variant thereof) is a nickase that has been modified (e.g., incorporates one or more amino acid modifications compared to a reference sequence) to impart nickase activity. For example, the DNA binding nickase (or a functional fragment or variant thereof) may be a Cas9 nuclease (or functional fragment or variant thereof) with one of the two nuclease domains inactivated, e.g., by amino acid substitution of H840A, wherein the Cas9 has nickase activity but is not able to make a double strand break in a target double stranded polynucleotide.

In some embodiments, the DNA binding nickase comprises a Cas9 nickase, Cas12e (CasX) nickase, Cas12d (CasY) nickase, Cas12a (Cpf1) nickase, Cas12b1 (C2c1) nickase, Cas13a (C2c2) nickase, Cas12c (C2c3) nickase (or a functional fragment or variant of any of the foregoing).

In some embodiments, the DNA binding nickase is a Cas9 nickase (or a functional fragment or variant thereof). The wild type Cas9 comprises two separate nuclease domains, the RuvC domain (which cleaves the non-protospacer DNA strand) and HNH domain (which cleaves the protospacer DNA strand). In some embodiments, the Cas9 nickase comprises only a single functioning nuclease domain.

In some embodiments, the Cas9 nickase comprises a mutation in the RuvC domain which inactivates the RuvC nuclease activity. Suitable mutations include, but are not limited to, e.g., in aspartate (D) 10, histidine (H) 983, aspartate (D) 986, or glutamate (E) 762, (See, e.g., Nishimasu et al., “Crystal structure of Cas9 in complex with guide RNA and target DNA,” Cell/ 156(5), 935-949, which is incorporated herein by reference). In some embodiments, the Cas9 nickase (or a functional fragment or variant thereof) comprises at least one of the following amino acid substitutions D10X, H983X, D986X, or E762X, wherein X is any amino acid other than the wild-type amino acid. In some embodiments, the Cas9 nickase (or a functional fragment or variant thereof) comprises at least one of the following amino acid substitutions D10A, H983A, D986A, or E762A, or a combination thereof. A Cas9 nickase (or a functional fragment or variant thereof) comprising a D10A amino acid substitution is also referred to herein as Cas9-D10A. Likewise, a Cas9 nickase (or a functional fragment or variant thereof) comprising a H983A amino acid substitution is also referred to herein as Cas9-H983A. A Cas9 nickase (or a functional fragment or variant thereof) comprising a D986A amino acid substitution is also referred to herein as Cas9-D986A. A Cas9 nickase (or a functional fragment or variant thereof) comprising a E762A amino acid substitution is also referred to herein as Cas9-E762A.

In some embodiments, the Cas9 nickase (or a functional fragment or variant thereof) comprises a mutation in the HNH domain which inactivates the HNH nuclease activity. Suitable mutations include, but are not limited to, a mutation in histidine (H) 840 or asparagine (R) 863 (amino acid numbering relative to SEQ ID NO: 1) (See supra). In some embodiments, the Cas9 nickase (or a functional fragment or variant thereof) comprises at least one of the following amino acid substitutions H840X or R863X, wherein X is any amino acid other than the wild-type amino acid. In some embodiments, the Cas9 nickase (or a functional fragment or variant thereof) comprises at least one of the following amino acid substitutions H840A or R863A, or a combination thereof. A Cas9 nickase (or a functional fragment or variant thereof) comprising an H840A amino acid substitution is also referred to herein as Cas9-H840A. Likewise, a Cas9 nickase (or a functional fragment or variant thereof) comprising an R863A amino acid substitution is also referred to herein as a Cas9-R863A.

In some embodiments, the DNA binding nickase (or a functional fragment or variant thereof) comprises Cas9-D10A, Cas9-H983A, Cas9-D986A, Cas9-E762A, Ca9s-H840A, or Cas9-R863A (or a functional fragment or variant of any of the foregoing). In some embodiments, the DNA binding nickase (or a functional fragment or variant thereof) comprises Cas9-D10A, Cas9-H983A, Cas9-D986A, or Cas9-E762A (or a functional fragment or variant of any of the foregoing). In some embodiments, the DNA binding nickase comprises Cas9-H840A or Cas9-R863A (or a functional fragment or variant of any of the foregoing). In some embodiments, the DNA binding nickase (or a functional fragment or variant thereof) comprises Cas9-H840A (or a functional fragment or variant of any of the foregoing).

Reverse Transcriptases

In some embodiments, the compositions, systems, and methods described herein utilize a reverse transcriptase (or a functional fragment or variant thereof). In some embodiments, a functional fragment or functional variants of a reverse transcriptase is used, wherein the fragment or variant maintains reverse transcriptase activity.

In some embodiments, the reverse transcriptase is a naturally occurring reverse transcriptase (or functional fragment or variant thereof). In some embodiments, the reverse transcriptase is derived from a naturally occurring reverse transcriptase (or functional fragment or variant thereof). In some embodiments, the reverse transcriptase (or a functional fragment or variant thereof) is a reverse transcriptase that has been modified (e.g., incorporates one or more amino acid modifications compared to a reference sequence). In some embodiments, the modified reverse transcriptase comprises one or more improved properties as compared to the corresponding reference sequence (e.g., thermostability, fidelity, reverse transcriptase activity).

Exemplary reverse transcriptases include, but are not limited to, moloney murine leukemia virus (M-MLV) reverse transcriptase; human immunodeficiency virus (HIV) reverse transcriptase and avian sarcoma-leukosis virus (ASLV) reverse transcriptase, which includes but is not limited to rous sarcoma virus (RSV) reverse transcriptase, avian myeloblastosis virus (AMY) reverse transcriptase, avian erythroblastosis virus (AEV) helper virus MCAV reverse transcriptase, avian myelocytomatosis virus MC29 helper virus MCAV reverse transcriptase, avian reticuloendotheliosis virus (REV-T) helper virus REV-A reverse transcriptase, avian sarcoma virus UR2 helper virus UR2AV reverse transcriptase, avian sarcoma virus Y73 helper virus YAV reverse transcriptase, rous associated virus (RAV) reverse transcriptase, and myeloblastosis associated virus (MAV) reverse transcriptase.

Any of the forementioned exemplary reverse transcriptases can be modified, e.g., comprises at least one amino acid substitution, deletion, or addition.

In some embodiments, the reverse transcriptase is derived from the M-MLV reverse transcriptase. In some embodiments, the M-MLV reverse transcriptase is naturally occurring. In some embodiments, the M-MLV reverse transcriptase is non-naturally occurring.

### 7.4. Integrases

In some embodiments, the compositions, systems, and methods described herein utilize an integrase (or a functional fragment or variant thereof) and a cognate integration sequence. Integrases, integration sequences, and integration sites are particularly useful in methods of PASTE editing (e.g., as described herein). It is understood by the person of ordinary skill in the art that integration sites and integrases for use in the compositions, systems, and methods described herein will be selected in pairs, wherein the selected integrase will specifically recognize the selected integration site.

The integrase (or functional fragment or variant thereof) can be provided as part of the editing polypeptide (e.g., as described herein, e.g., as a fusion protein) or as a separate polypeptide. In some embodiments, the integrase (or functional fragment or variant thereof) is part of the editing polypeptide (e.g., a fusion protein). In some embodiments, the integrase (or functional fragment or variant thereof) is polypeptide separate from the editing polypeptide.

Exemplary integrases include recombinases, reverse transcriptases, and retrotransposases. Exemplary integrases include, but are not limited to, Cre, Dre, Vika, Bxb1, φC31, RDF, FLP, φBT1, R1, R2, R3, R4, R5, TP901-1, A118, φFC1, φC1, MR11, TG1, φ370.1, WO, BL3, SPBc, K38, Peaches, Veracruz, Rebeuca, Theia, Benedict, KSSJEB, PattyP, Doom, Scowl, Lockley, Switzer, Bob3, Troube, Abrogate, Anglerfish, Sarfire, SkiPole, Conceptll, Museum, Severus, Airmid, Benedict, Hinder, ICleared, Sheen, Mundrea, BxZ2, φRV, and retrotransposases encoded by R2, L1, To12 Tc1, Tc3, Mariner (Himar 1), Mariner (mos 1), and Minos. In some embodiments, the integrase is Bxb1.

The integrases (e.g., recombinases) explicitly provided herein are not meant to be exclusive examples of integrases (e.g., recombinases) that can be used in embodiments of the disclosure. The methods and compositions of the disclosure can be expanded by mining databases for new orthogonal integrases (e.g., recombinases) or designing synthetic integrases (e.g., recombinases) with defined DNA specificities (See, e.g., Groth et al., “Phage integrases: biology and applications.” J. Mol. Biol. 2004; 335, 667-678; Gordley et al., “Synthesis of programmable integrases.” Proc. Natl. Acad. Sci. USA. 2009; 106, 5053-5058; the entire contents of each of which is hereby incorporated by reference in their entirety for all purposes).

In some embodiments, the integrase (or functional fragment or variant thereof) is a recombinase that incorporates the polynucleotide of interest into the target polynucleotide (e.g., a genome of a cell) at an integration site by recombination. Exemplary recombinases include serine recombinases and tyrosine recombinases. In some embodiments, the integrase is a serine recombinase. In some embodiments, the integrase is a tyrosine recombinase. Exemplary serine recombinases include, but are not limited to, Hin, Gin, Tn3, β-six, CinH, ParA, γδ, Bxb 1, φC31, TP901, TG1, φBT1, R1, R2, R3, R4, R5, φRV1, φFC1, MR11, A118, U153, gp29. Examples of serine recombinases also include, without limitation, recombinases Peaches, Veracruz, Rebeuca, Theia, Benedict, KSSJEB, PattyP, Doom, Scowl, Lockley, Switzer, Bob3, Troube, Abrogate, Anglerfish, Sarfire, SkiPole, Conceptll, Museum, Severus, Airmid, Benedict, Hinder, ICleared, Sheen, Mundrea, and BxZ2 from Mycobacterial phages. In some embodiments, the integrase is Hin, Gin, Tn3, β-six, CinH, ParA, γδ, Bxb1, φC31, TP901, TG1, φBT1, R1, R2, R3, R4, R5, φRV1, φFC1, MR11, A118, U153, or gp29. In some embodiments, the integrase is a tyrosine recombinase. Exemplary, tyrosine recombinases include, but are not limited to, Cre, FLP, R, Lambda, HK101, HK022, and pSAM2.

In some embodiments, the integrase is a reverse transcriptase that incorporates the polynucleotide of interest into the target polynucleotide (e.g., a genome of a cell) at an integration site by reverse transcription.

In some embodiments, the integrase (or functional fragment or variant thereof) is a retrotransposase that incorporates the polynucleotide of interest into the target polynucleotide (e.g., a genome of a cell) at an integration site by retrotransposition. Exemplary retrotransposases include, but are not limited to, retrotransposases encoded by elements such as R2, L1, To12 Tc1, Tc3, Mariner (Himar 1), Mariner (mos 1), Minos, and any functional variants thereof.

### 7.5. Linkers

In some embodiments, the compositions, systems, and methods described herein utilize a linker (e.g., a peptide linker) (e.g., one or more different linkers). Common linkers (e.g., glycine and glycine/serine linkers) are known in the art. Any suitable linker(s) can be utilized as long as each component can mediate the desired function.

In some embodiments, at least two components of an editing polypeptide (e.g., described herein) are operably connected via a linker. In some embodiments, each component of an editing polypeptide (e.g., described herein) is operably connected to the preceding and/or subsequent component of the editing polypeptide via a linker. In some embodiments, each component of an editing polypeptide (e.g., described herein) is operably connected to the preceding and/or subsequent component of the editing polypeptide via a different linker.

In some embodiments, the linker is from about 2-100, 2-50, 2-25, 2-10, 4-100, 4-4-25, 4-10, 5-100, 5-50, 5-25, 5-10, 10-100, 10-50, or 10-25 amino acids in length. In some embodiments, the linker is about 2, 3, 4, 5, 6, 7, 8, 9, 10, 11, 12, 13, 14, 15, 16, 17, 18, 19, 20, 30, 35, 40, 45, 50, 55, 60, 65, 70, 75, 80, 85, 90, 95, or 100 amino acids in length.

### 7.6. Reverse Transcriptase Template Sequence

In some embodiments, the compositions, systems, and methods described herein utilize a reverse transcriptase template sequence. The reverse transcriptase template sequence serves as a template (i.e., encodes) the polynucleotide of interest (e.g., polynucleotide comprising, e.g., therapeutic nucleotide modification, diagnostic nucleotide modification; or e.g., a polynucleotide comprising an integration sequence encoding an integration site) for incorporation into a target polynucleotide (e.g., a gene or genome of a cell). In some embodiments, the reverse transcriptase template sequence comprises a therapeutic or diagnostic target nucleotide modification (e.g., in some embodiments a single nucleotide substitution, e.g., for use in PRIME editing methods). In some embodiments, the reverse transcriptase template sequence comprises an integration sequence comprising an integration site.

### 7.7. Integration Sequences and Integration Sites

In some embodiments, the compositions, systems, and methods described herein utilize an integration sequence (e.g., comprising an integration site) and a cognate integrase (e.g., as described herein). Integration sequences, integration sites, and integrases are particularly useful in methods of PASTE editing (e.g., as described herein). In some embodiments, the gRNA comprises an integration sequence encoding an integration site. Inclusion of the integration sequence encoding an integration site in the gRNA allows for the incorporation of the integration site into a desired (site-specific) location in the polynucleotide (e.g., gene or genome) being edited.

It is understood by the person of ordinary skill in the art that integration sites and integrases for use in the compositions, systems, and methods described herein will be selected in pairs, wherein the selected integrase will specifically recognize the selected integration site. Exemplary integration sites include, but are not limited to, lox71 sites, attB sites, attP sites, attL sites, attR sites, Vox sites, FRT sites, or pseudo attP sites.

It is common knowledge to the person of ordinary skill in the art, that integration typically requires (e.g., as with serine integrases) an integration site (encoded by the gRNA) and a recognition site (e.g., linked to a polynucleotide of interest for insertion) both of which are recognized by the integrase. The integration site can be inserted into the target polynucleotide (e.g., of a cell) using a nuclease (e.g., a nickase), a gRNA, and/or an integrase. A single or a plurality of integration sites can be added to a target polynucleotide (e.g., a genome). In some embodiments, one integration site is added to a target polynucleotide (e.g., a genome). In some embodiments, more than one integration site is added to a target polynucleotide (e.g., a genome). The recognition site may be operably linked to a target polynucleotide (e.g., gene of interest) in an exogenous DNA or RNA (e.g., as described herein).

To insert more than one unique polynucleotide (e.g., gene) of interest, each at a specific site, multiple orthogonal integrations sites can be added to the specific desired locations or target sites within the polynucleotide (e.g., genome) to mediate site-specific integration of the multiple polynucleotides. A first integration site is “orthogonal” to a second integration site when it does not significantly recognize the recognition site or the integrase (e.g., recombinase) recognized by the second integration site. Thus, for example, one attB site of an integrase (e.g., a recombinase) can be orthogonal to an attB site of a different recombinase (e.g., integrase). In addition, one pair of attB and attP sites of an integrase (e.g., a recombinase) can be orthogonal to another pair of attB and attP sites recognized by the same integrase (e.g., recombinase). A pair of recombinases are considered orthogonal to each other, as defined herein, when there is recognition of each other's attB or attP site sequences. In some embodiments, the same integrase (e.g., recombinase) or two different recombinases (e.g., integrases) recognize the same integration site less than 30%, 28%, 26%, 24%, 22%, 20%, 18%, 16%, 14%, 12%, 10%, 8%, 6%, 4%, 2%, or 1%, or any range that is formed from any two of those values as endpoints of the time.

A single or a plurality of integration sites can be added to a target polynucleotide (e.g., a genome). In some embodiments, one integration site is added to a target polynucleotide (e.g., a genome). In some embodiments, more than one integration site is added to a target polynucleotide (e.g., a genome).

The central dinucleotide of some integrases is involved in the association of the two paired integration sites. For example, the central dinucleotide of BxbINT is involved in the association of the AttB integration site with the AttP recognition site. Therefore, changing the matched central dinucleotide can modify the integrase activity and provide orthogonality for the insertion of multiple genes. Therefore, expanding the set of AttB/AttP dinucleotides can enable multiplex gene insertion using gRNAs.

In some embodiments, the attB and/or attP site sequences comprise a central dinucleotide sequence. It has been shown that, for example, the central dinucleotide can be changed to GA from GT and that only GA containing attB/attP sites interact and will not cross react with GT containing sequences. In some embodiments, the central dinucleotide is selected from the group consisting of AG, AC, TG, TC, CA, CT, GA, AA, TT, CC, GG, AT, TA, GC, CG and GT. In some embodiments, the central dinucleotide is nonpalindromic. In some embodiments, the central dinucleotide is palindromic. In some embodiments, the integration site and the recognition site of a pair share the same central dinucleotide and can mediate recombination in the presence of the cognate integrase.

7.8. gRNAs

In some embodiments, the compositions, systems, and methods described herein comprise or utilize a gRNA. A gRNA typically functions to guide the insertion or deletion of one or more polynucleotides of interest (e.g., a gene of interest) into a target polynucleotide (e.g., genome). In some embodiments, the gRNA molecule is naturally occurring. In some embodiments, a gRNA molecule is non-naturally occurring. In some embodiments, a gRNA molecule is a synthetic gRNA molecule. In some embodiments, the gRNA comprises one or nucleotide modifications (e.g., to improve stability and/or half-life after being introduced into a cell).

7.9. Paired gRNAs

In some embodiments, the compositions, systems, and methods described herein comprise or utilize one or more set of paired guides that allow for the simultaneous deletion of an endogenous polynucleotide (e.g., gene) and insertion of a polynucleotide of interest (e.g., modified gene). The target dsDNA comprises two protospacers each on opposite strands of the target dsDNA. One gRNA (e.g., targeting gRNA) is targeted to one strand, while the other gRNA (e.g., targeting gRNA) of the pairs is targeted to the opposite strand. The targeting gRNA: editing polypeptide complex generates a single strand nick at each target site.

7.10. Modification of gRNAs

In some embodiments, the gRNA comprises one or nucleotide modifications (e.g., to improve stability and/or half-life after being introduced into a cell). In some embodiments, chemical modifications on the ribose rings and phosphate backbone of gRNAs are incorporated. Ribose modifications are typically placed at the 2′OH as it is readily available for manipulation. Simple modifications at the 2′OH include 2′-O-methyl, 2′-fluoro, and 2′-deoxy-2′-fluoro-beta-D-arabinonucleic acid (2′fluoro-ANA). More extensive ribose modifications such as 2′F-4′-Cα-OMe and 2′,4′-di-Cα-OMe combine modification at both the 2′ and 4′ carbons. Exemplary phosphodiester modifications include sulfide-based phosphorothioate (PS) or acetate-based phosphonoacetate alterations. Combinations of the ribose and phosphodiester modifications can also be utilized such as 2′-O-methyl 3′phosphorothioate (MS), or 2′-O-methyl-3′-thioPACE (MSP), and 2′-O-methyl-3′-phosphonoacetate (MP) RNAs. Locked and unlocked nucleotides such as locked nucleic acid (LNA), bridged nucleic acids (BNA), S-constrained ethyl (cEt), and unlocked nucleic acid (UNA) are examples of sterically hindered nucleotide modifications that can also be utilized.

7.11. Delivery of gRNAs

The gRNAs described herein (e.g., targeting gRNAs, ngRNAs) can be delivered to a cell or a population of cells by any suitable method known in the art. For example, via an RNA polynucleotide; via a vector (e.g., a plasmid or viral vector) comprising an RNA polynucleotide; via a particle (e.g., a viral particle, lipid particle, nanoparticle (e.g., a lipid nanoparticle)) encapsulating the polynucleotide or vector. Methods of delivering each of the aforementioned are known to the person of ordinary skill in the art. Also provided herein are pharmaceutical compositions comprising a gRNA described herein (e.g., targeting gRNA, ngRNA) polynucleotide; a vector (e.g., a plasmid or viral vector) comprising the polynucleotide; a particle (e.g., a viral particle, lipid particle, nanoparticle (e.g., a lipid nanoparticle)) encapsulating the polynucleotide; and a pharmaceutically acceptable excipient.

Exemplary viral vectors include, but are not limited to, adenovirus vectors, adeno-associated virus vectors, lentivirus vectors, retrovirus vectors, poxvirus vectors, parapoxivirus vectors, vaccinia virus vectors, fowlpox virus vectors, herpes virus vectors, adeno-associated virus vectors, alphavirus vectors, lentivirus vectors, rhabdovirus vectors, measles virus, Newcastle disease virus vectors, picornaviruses vectors, or lymphocytic choriomeningitis virus vectors.

### 7.12. Compositions, Pharmaceutical Compositions, Systems, and Kits

Provided herein are compositions (including pharmaceutical compositions), systems, and kits comprising any one or more (e.g., all) of the components described herein (e.g., an editing polypeptide, one of more gRNAs, polynucleotide inserts). In one aspect, provided herein is a system comprising at least two components of an editing system described herein (e.g., a DNA binding nickase, a reverse transcriptase, a integration enzyme, a gRNA pair). In one aspect, provided herein are compositions comprising at least one components of an editing system described herein (e.g., a DNA binding nickase, a reverse transcriptase, a integration enzyme, a gRNA pair).

### 7.13. Pharmaceutical Compositions

Pharmaceutical compositions descried herein comprise at least one component of an editing system described herein (e.g., a DNA binding nickase) and a pharmaceutically acceptable excipient (see, e.g., Remington's Pharmaceutical Sciences (1990) Mack Publishing Co., Easton, PA, the entire contents of which is incorporated by reference herein for all purposes).

In one aspect, also provided herein are methods of making pharmaceutical compositions described herein comprising providing at least one component of an editing system described herein (e.g., a DNA binding nickase) and formulating it into a pharmaceutically acceptable composition by the addition of one or more pharmaceutically acceptable excipient. In some embodiments, the pharmaceutical composition comprises a single component described herein (e.g., a DNA binding nickase). In some embodiments, the pharmaceutical composition comprises a plurality of the components described herein (e.g., a DNA binding nickase, a reverse transcriptase, a integration enzyme, a gRNA pair, etc.).

Acceptable excipients (e.g., carriers and stabilizers) are preferably nontoxic to recipients at the dosages and concentrations employed, and include buffers such as phosphate, citrate, or other organic acids; antioxidants including ascorbic acid or methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol;or m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, or other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

A pharmaceutical composition may be formulated for any route of administration to a subject. The skilled person knows the various possibilities to administer a pharmaceutical composition described herein a in order to deliver the editing system or composition to a target cell. Non-limiting embodiments include parenteral administration, such as intramuscular, intradermal, subcutaneous, transcutaneous, or mucosal administration. In one embodiment, the pharmaceutical composition is formulated for intravenous administration. In one embodiment, the pharmaceutical composition is formulated for administration by intramuscular, intradermal, or subcutaneous injection. Injectables can be prepared in conventional forms, either as liquid solutions or suspensions. The injectables can contain one or more excipients. Exemplary excipients include, for example, water, saline, dextrose, glycerol or ethanol. In addition, if desired, the pharmaceutical compositions to be administered can also contain minor amounts of non-toxic auxiliary substances such as wetting or emulsifying agents, pH buffering agents, stabilizers, solubility enhancers, or other such agents, such as for example, sodium acetate, sorbitan monolaurate, triethanolamine oleate or cyclodextrins. In some embodiments, the pharmaceutical composition is formulated in a single dose. In some embodiments, the pharmaceutical compositions if formulated as a multi-dose.

Pharmaceutically acceptable excipients (e.g., carriers) used in the parenteral preparations described herein include for example, aqueous vehicles, nonaqueous vehicles, antimicrobial agents, isotonic agents, buffers, antioxidants, local anesthetics, suspending and dispersing agents, emulsifying agents, sequestering or chelating agents or other pharmaceutically acceptable substances. Examples of aqueous vehicles, which can be incorporated in one or more of the formulations described herein, include sodium chloride injection, Ringer's injection, isotonic dextrose injection, sterile water injection, dextrose or lactated Ringer's injection. Nonaqueous parenteral vehicles, which can be incorporated in one or more of the formulations described herein, include fixed oils of vegetable origin, cottonseed oil, corn oil, sesame oil or peanut oil. Antimicrobial agents in bacteriostatic or fungistatic concentrations can be added to the parenteral preparations described herein and packaged in multiple-dose containers, which include phenols or cresols, mercurials, benzyl alcohol, chlorobutanol, methyl and propyl p-hydroxybenzoic acid esters, thimerosal, benzalkonium chloride or benzethonium chloride. Isotonic agents, which can be incorporated in one or more of the formulations described herein, include sodium chloride or dextrose. Buffers, which can be incorporated in one or more of the formulations described herein, include phosphate or citrate. Antioxidants, which can be incorporated in one or more of the formulations described herein, include sodium bisulfate. Local anesthetics, which can be incorporated in one or more of the formulations described herein, include procaine hydrochloride. Suspending and dispersing agents, which can be incorporated in one or more of the formulations described herein, include sodium carboxymethylcelluose, hydroxypropyl methylcellulose or polyvinylpyrrolidone. Emulsifying agents, which can be incorporated in one or more of the formulations described herein, include Polysorbate 80 (TWEEN® 80). A sequestering or chelating agent of metal ions, which can be incorporated in one or more of the formulations described herein, is EDTA. Pharmaceutical carriers, which can be incorporated in one or more of the formulations described herein, also include ethyl alcohol, polyethylene glycol or propylene glycol for water miscible vehicles; orsodium hydroxide, hydrochloric acid, citric acid or lactic acid for pH adjustment.

The precise dose to be employed in a pharmaceutical composition will also depend on the route of administration, and the seriousness of the condition caused by it, and should be decided according to the judgment of the practitioner and each subject's circumstances. For example, effective doses may also vary depending upon means of administration, target site, physiological state of the subject (including age, body weight, and health), other medications administered, or whether therapy is prophylactic or therapeutic. Therapeutic dosages are preferably titrated to optimize safety and efficacy.

### 7.14. Kits

Also provided herein are kits comprising at least one pharmaceutical composition described herein. In addition, the kit may comprise a liquid vehicle for solubilizing or diluting, and/or technical instructions. The technical instructions of the kit may contain information about administration and dosage and subject groups. In some embodiments, the kit contains a single container comprising a single pharmaceutical composition described herein. In some embodiments, the kit at least two separate containers, each comprising a different pharmaceutical composition described herein (e.g., a first container comprising a pharmaceutical composition comprising one component of an editing system described herein, e.g., an editing polypeptide described herein, and a second container comprising a second pharmaceutical composition comprising a second component of an editing system described herein, e.g., a gRNA).

### EXAMPLES

### Example 1

### Design and Construction of Paired Guides

Guide RNA (gRNA) pairs comprising two heterologous atgRNAs for gene editing were assessed.

The gRNA pairs were used to replace the pegRNA and nicking guide generally found in PASTE system to more efficiently introduce long PASTE sequence edits (38-46 bp). The two heterologous atgRNAs comprise three design considerations which are tested in Example 2 below: (1) the spacing between both atgRNA relative to each other, (2) the different combinations of guides, and (3) the amount of overlap between the attB insertion site of the two guides.

Although complete overlap via complementary sequence of the two atgRNA results in gene insertion, incomplete overlap (for example, 14 bp to about 46 bp of site overlap) can enhance insertion efficiency. For example, incomplete overlap of the attB integration sequence with respect to the first and second heterologous gRNAs may prevent off-target integration into guide plasmids. Furthermore, no nicking guide is needed when gRNA pairs are used. The nicking guide is replaced by engineered spacer sequences in of both atgRNAs. Moreover, the reverse transcriptase (RT) is optional and according to the examples presented below removing the RT can yield better performing paired guides.

Table 1 below lists exemplary sequences for some of the PASTE system elements (integration site sequence and scaffold).

TABLE A
Nucleic acid encoding PASTE system
elements-integration site

| Description | Nucleic acid sequence |
|---|---|
| AttP | GTGGTTTGTCTGGTCAACCACCGCGG |
| integration | TCTCAGTGGTGTACGGTACAAACCCA |
| site 1 | (SEQ ID NO: 395) |
| AttP | GGTTTGTCTGGTCAACCACCGCGGTC |
| integration | TCAGTGGTGTACGGTACAAACC |
| site 2- | (SEQ ID NO: 396) |
| Twin PE | |

TABLE B
Nucleic acid encoding PASTE system
elements-Scaffold

| Description | Nucleic acid sequence |
|---|---|
| Standard | Gttttagagctagaaatagcaagtt |
| scaffold | aaaataaggctagtccgttatcaac |
| | ttgaaaaagtggcaccgagtcggtg |
| | c |
| | (SEQ ID NO: 397) |
| Optimized | Gttttagagctagaaatagcaagtt |
| scaffold | aaaataaggctagtccgttatcaac |
| | ttgaaaaagtggcaccgagtcggtg |
| | c |
| | (SEQ ID NO: 397) |

### 8.2. Example 2

### Screen of Paired Guides Functioning With PASTE

Different gRNA pair designs based on the design considerations presented in Example 1 were assessed, by analyzing the attb attachment site integration efficiency was assessed as well.

Panels of paired guides were designed with specificity for the ACTB, mouse DNMT1, and mouse NOLC1 locus, corresponding to paired guide sequences shown below in Table 1, 2, and 3 respectively.

### Material and Methods—ACTB Locus

Cell culture. HEK293FT cells (American Type Culture Collection (ATCC)-CRL32156) were cultured in Dulbecco's Modified Eagle Medium with high glucose, sodium pyruvate, and GlutaMAX (Thermo Fisher Scientific), additionally supplemented with 10% (v/v) fetal bovine serum (FBS) and 1× penicillin-streptomycin (Thermo Fisher Scientific).

Transfection. Cells were plated at 5-15K the day prior to transfection in a 96-well plate coated with poly-D-lysine (BD Biocoat). HEK293FT were transfected with Lipofectamine 3000 (Thermo Fisher Scientific), according to manufacturer's specifications. For AttB insertion, 35.5ng of each dual guide plasmid and 100 ng SpCas9-RT plasmid were delivered to each well.

Genomic DNA extraction, purification, and quantitation. DNA was harvested from transfected cells by removal of media, resuspension in 50 μL of QuickExtract (Lucigen), and incubation at 65° C. for 15 min, 68° C. for 15 min, and 98° C. for 10 min. Target regions were PCR amplified with NEBNext High-Fidelity 2× PCR Master Mix (NEB) based on the manufacturer's protocol. Barcodes and adapters for Illumina sequencing were added in a subsequent PCR amplification. Amplicons were pooled and prepared for sequencing on a MiSeq (Illumina). Reads were demultiplexed and analyzed with appropriate pipelines.

### Results—ACTB Locus

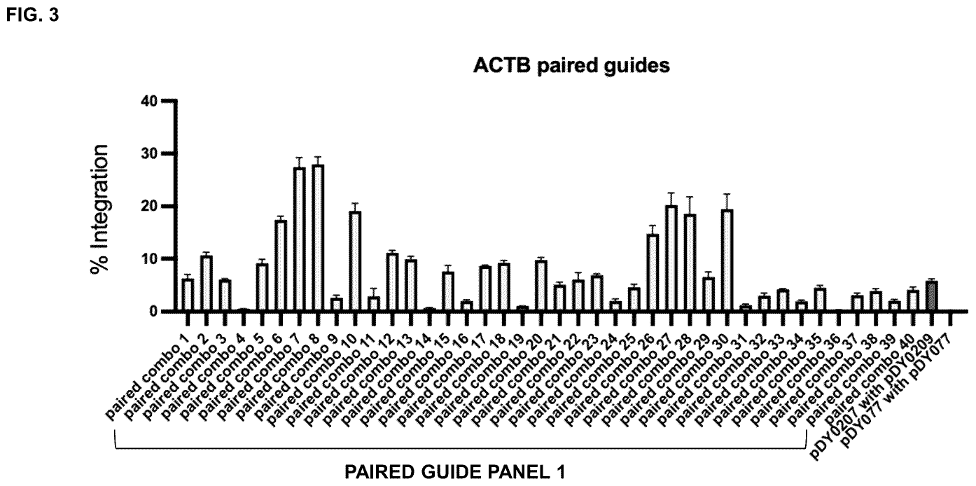

Specific ACTB specific paired guides matched or exceeded the percent attB integration efficiency relative to functioned at a significant yield with multiple pairs matching or exceeding single guide performance (FIG. 3). Accordingly, paired guides can enable more rapid screening techniques of much larger design spaces.

TABLE 1
Nucleic acid encoding Paired Guides for AttB insertion at the ACTB locus

| | | SEQ | | SEQ |
|---|---|---|---|---|
| Pairing | Nucleic Acid Guide | ID | Nucleic Acid Guide | ID |
| Combo | Sequence 1 | NO | Sequence 2 | NO |
| 1 | gACCTCGGCTCACAGCG | 1 | GAAGCCGGCCTTGCACAT | 2 |
| | CGCCgttttagagctagaaatagca | | GCgttttagagctagaaatagcaagttaa | |
| | agttaaaataaggctagtccgttatcaa | | aataaggctagtccgttatcaacttgaaaa | |
| | cttgaaaaagtggcaccGAGTCG | | agtggcaccGAGTCGGTGCGG | |
| | GTGCccggatgatcctgacgacg | | CCGGCTTGTCGACGACGG | |
| | gagaccgccgtcgtcgacaagccgg | | CGGTCTCCGTCGTCAGGA | |
| | ccgcgctgtgagccg | | TCATCCGGtgtgcaaggccgg | |
| 2 | gACCTCGGCTCACAGCG | 3 | GGCATCGTCGCCCGCGAA | 4 |
| | CGCCgttttagagctagaaatagca | | GCgttttagagctagaaatagcaagttaa | |
| | agttaaaataaggctagtccgttatcaa | | aataaggctagtccgttatcaacttgaaaa | |
| | cttgaaaaagtggcaccGAGTCG | | agtggcaccGAGTCGGTGCGG | |
| | GTGCccggatgatcctgacgacg | | CCGGCTTGTCGACGACGG | |
| | gagaccgccgtcgtcgacaagccgg | | CGGTCTCCGTCGTCAGGA | |
| | ccgcgctgtgagccg | | TCATCCGGtcgcgggcgacga | |
| 3 | gACCTCGGCTCACAGCG | 5 | GGAGGGGAAGACGGCCC | 6 |
| | CGCCgttttagagctagaaatagca | | GGGgttttagagctagaaatagcaagtt | |
| | agttaaaataaggctagtccgttatcaa | | aaaataaggctagtccgttatcaacttgaa | |
| | cttgaaaaagtggcaccGAGTCG | | aaagtggcaccGAGTCGGTGCG | |
| | GTGCccggatgatcctgacgacg | | GCCGGCTTGTCGACGACG | |
| | gagaccgccgtcgtcgacaagccgg | | GCGGTCTCCGTCGTCAGG | |
| | ccgcgctgtgagccg | | ATCATCCGGgggccgtcttccc | |
| 4 | gACCTCGGCTCACAGCG | 7 | gTCTTCCCCTCCATCGTGG | 8 |
| | CGCCgttttagagctagaaatagca | | GGgttttagagctagaaatagcaagttaa | |
| | agttaaaataaggctagtccgttatcaa | | aataaggctagtccgttatcaacttgaaaa | |
| | cttgaaaaagtggcaccGAGTCG | | agtggcaccGAGTCGGTGCGG | |
| | GTGCccggatgatcctgacgacg | | CCGGCTTGTCGACGACGG | |
| | gagaccgccgtcgtcgacaagccgg | | CGGTCTCCGTCGTCAGGA | |
| | ccgcgctgtgagccg | | TCATCCGGcacgatggagggg | |
| 5 | gACCTCGGCTCACAGCG | 9 | gCTGGGGCGCCCCACGAT | 10 |
| | CGCCgttttagagctagaaatagca | | GGAgttttagagctagaaatagcaagtt | |
| | agttaaaataaggctagtccgttatcaa | | aaaataaggctagtccgttatcaacttgaa | |
| | cttgaaaaagtggcaccGAGTCG | | aaagtggcaccGAGTCGGTGCG | |
| | GTGCccggatgatcctgacgacg | | GCCGGCTTGTCGACGACG | |
| | gagaccgccgtcgtcgacaagccgg | | GCGGTCTCCGTCGTCAGG | |
| | ccgcgctgtgagccg | | ATCATCCGGatcgtggggcgcc | |
| 6 | GCTATTCTCGCAGCTCA | 11 | GAAGCCGGCCTTGCACAT | 12 |
| | CCAgttttagagctagaaatagcaa | | GCgttttagagctagaaatagcaagttaa | |
| | gttaaaataaggctagtccgttatcaac | | aataaggctagtccgttatcaacttgaaaa | |
| | ttgaaaaagtggcaccGAGTCG | | agtggcaccGAGTCGGTGCGG | |
| | GTGCccggatgatcctgacgacg | | CCGGCTTGTCGACGACGG | |
| | gagaccgccgtcgtcgacaagccgg | | CGGTCTCCGTCGTCAGGA | |
| | cctgagctgcgagaa | | TCATCCGGtgtgcaaggccgg | |
| 7 | GCTATTCTCGCAGCTCA | 13 | GGCATCGTCGCCCGCGAA | 14 |
| | CCAgttttagagctagaaatagcaa | | GCgttttagagctagaaatagcaagttaa | |
| | gttaaaataaggctagtccgttatcaac | | aataaggctagtccgttatcaacttgaaaa | |
| | ttgaaaaagtggcaccGAGTCG | | agtggcaccGAGTCGGTGCGG | |
| | GTGCccggatgatcctgacgacg | | CCGGCTTGTCGACGACGG | |
| | gagaccgccgtcgtcgacaagccgg | | CGGTCTCCGTCGTCAGGA | |
| | cctgagctgcgagaa | | TCATCCGGtcgcgggcgacga | |
| 8 | GCTATTCTCGCAGCTCA | 15 | GGAGGGGAAGACGGCCC | 16 |
| | CCAgttttagagctagaaatagcaa | | GGGgttttagagctagaaatagcaagtt | |
| | gttaaaataaggctagtccgttatcaac | | aaaataaggctagtccgttatcaacttgaa | |
| | ttgaaaaagtggcaccGAGTCG | | aaagtggcaccGAGTCGGTGCG | |
| | GTGCccggatgatcctgacgacg | | GCCGGCTTGTCGACGACG | |
| | gagaccgccgtcgtcgacaagccgg | | GCGGTCTCCGTCGTCAGG | |
| | cctgagctgcgagaa | | ATCATCCGGgggccgtcttccc | |
| 9 | GCTATTCTCGCAGCTCA | 17 | gTCTTCCCCTCCATCGTGG | 18 |
| | CCAgttttagagctagaaatagcaa | | GGgttttagagctagaaatagcaagttaa | |
| | gttaaaataaggctagtccgttatcaac | | aataaggctagtccgttatcaacttgaaaa | |
| | ttgaaaaagtggcaccGAGTCG | | agtggcaccGAGTCGGTGCGG | |
| | GTGCccggatgatcctgacgacg | | CCGGCTTGTCGACGACGG | |
| | gagaccgccgtcgtcgacaagccgg | | CGGTCTCCGTCGTCAGGA | |
| | cctgagctgcgagaa | | TCATCCGGcacgatggagggg | |
| 10 | GCTATTCTCGCAGCTCA | 19 | gCTGGGGCGCCCCACGAT | 20 |
| | CCAgttttagagctagaaatagcaa | | GGAgttttagagctagaaatagcaagtt | |
| | gttaaaataaggctagtccgttatcaac | | aaaataaggctagtccgttatcaacttgaa | |
| | ttgaaaaagtggcaccGAGTCG | | aaagtggcaccGAGTCGGTGCG | |
| | GTGCccggatgatcctgacgacg | | GCCGGCTTGTCGACGACG | |
| | gagaccgccgtcgtcgacaagccgg | | GCGGTCTCCGTCGTCAGG | |
| | cctgagctgcgagaa | | ATCATCCGGatcgtggggcgcc | |
| 11 | GCCGCGCTCGTCGTCG | 21 | GAAGCCGGCCTTGCACAT | 22 |
| | ACAAgttttagagctagaaatagca | | GCgttttagagctagaaatagcaagttaa | |
| | agttaaaataaggctagtccgttatcaa | | aataaggctagtccgttatcaacttgaaaa | |
| | cttgaaaaagtggcaccGAGTCG | | agtggcaccGAGTCGGTGCGG | |
| | GTGCccggatgatcctgacgacg | | CCGGCTTGTCGACGACGG | |
| | gagaccgccgtcgtcgacaagccgg | | CGGTCTCCGTCGTCAGGA | |
| | cctcgacgacgagcg | | TCATCCGGtgtgcaaggccgg | |
| 12 | GCCGCGCTCGTCGTCG | 23 | GGCATCGTCGCCCGCGAA | 24 |
| | ACAAgttttagagctagaaatagca | | GCgttttagagctagaaatagcaagttaa | |
| | agttaaaataaggctagtccgttatcaa | | aataaggctagtccgttatcaacttgaaaa | |
| | cttgaaaaagtggcaccGAGTCG | | agtggcaccGAGTCGGTGCGG | |
| | GTGCccggatgatcctgacgacg | | CCGGCTTGTCGACGACGG | |
| | gagaccgccgtcgtcgacaagccgg | | CGGTCTCCGTCGTCAGGA | |
| | cctcgacgacgagcg | | TCATCCGGtcgcgggcgacga | |
| 13 | GCCGCGCTCGTCGTCG | 25 | GGAGGGGAAGACGGCCC | 26 |
| | ACAAgttttagagctagaaatagca | | GGGgttttagagctagaaatagcaagtt | |
| | agttaaaataaggctagtccgttatcaa | | aaaataaggctagtccgttatcaacttgaa | |
| | cttgaaaaagtggcaccGAGTCG | | aaagtggcaccGAGTCGGTGCG | |
| | GTGCccggatgatcctgacgacg | | GCCGGCTTGTCGACGACG | |
| | gagaccgccgtcgtcgacaagccgg | | GCGGTCTCCGTCGTCAGG | |
| | cctcgacgacgagcg | | ATCATCCGGgggccgtcttccc | |
| 14 | GCCGCGCTCGTCGTCG | 27 | gTCTTCCCCTCCATCGTGG | 28 |
| | ACAAgttttagagctagaaatagca | | GGgttttagagctagaaatagcaagttaa | |
| | agttaaaataaggctagtccgttatcaa | | aataaggctagtccgttatcaacttgaaaa | |
| | cttgaaaaagtggcaccGAGTCG | | agtggcaccGAGTCGGTGCGG | |
| | GTGCccggatgatcctgacgacg | | CCGGCTTGTCGACGACGG | |
| | gagaccgccgtcgtcgacaagccgg | | CGGTCTCCGTCGTCAGGA | |
| | cctcgacgacgagcg | | TCATCCGGcacgatggagggg | |
| 15 | GCCGCGCTCGTCGTCG | 29 | gCTGGGGCGCCCCACGAT | 30 |
| | ACAAgttttagagctagaaatagca | | GGAgttttagagctagaaatagcaagtt | |
| | agttaaaataaggctagtccgttatcaa | | aaaataaggctagtccgttatcaacttgaa | |
| | cttgaaaaagtggcaccGAGTCG | | aaagtggcaccGAGTCGGTGCG | |
| | GTGCccggatgatcctgacgacg | | GCCGGCTTGTCGACGACG | |
| | gagaccgccgtcgtcgacaagccgg | | GCGGTCTCCGTCGTCAGG | |
| | cctcgacgacgagcg | | ATCATCCGGatcgtggggcgcc | |
| 16 | gCTCGTCGTCGACAACG | 31 | GAAGCCGGCCTTGCACAT | 32 |
| | GCTCgttttagagctagaaatagca | | GCgttttagagctagaaatagcaagttaa | |
| | agttaaaataaggctagtccgttatcaa | | aataaggctagtccgttatcaacttgaaaa | |
| | cttgaaaaagtggcaccGAGTCG | | agtggcaccGAGTCGGTGCGG | |
| | GTGCccggatgatcctgacgacg | | CCGGCTTGTCGACGACGG | |
| | gagaccgccgtcgtcgacaagccgg | | CGGTCTCCGTCGTCAGGA | |
| | ccccgttgtcgacga | | TCATCCGGtgtgcaaggccgg | |
| 17 | gCTCGTCGTCGACAACG | 33 | GGCATCGTCGCCCGCGAA | 34 |
| | GCTCgttttagagctagaaatagca | | GCgttttagagctagaaatagcaagttaa | |
| | agttaaaataaggctagtccgttatcaa | | aataaggctagtccgttatcaacttgaaaa | |
| | cttgaaaaagtggcaccGAGTCG | | agtggcaccGAGTCGGTGCGG | |
| | GTGCccggatgatcctgacgacg | | CCGGCTTGTCGACGACGG | |
| | gagaccgccgtcgtcgacaagccgg | | CGGTCTCCGTCGTCAGGA | |
| | ccccgttgtcgacga | | TCATCCGGtcgcgggcgacga | |
| 18 | gCTCGTCGTCGACAACG | 35 | GGAGGGGAAGACGGCCC | 36 |
| | GCTCgttttagagctagaaatagca | | GGGgttttagagctagaaatagcaagtt | |
| | agttaaaataaggctagtccgttatcaa | | aaaataaggctagtccgttatcaacttgaa | |
| | cttgaaaaagtggcaccGAGTCG | | aaagtggcaccGAGTCGGTGCG | |
| | GTGCccggatgatcctgacgacg | | GCCGGCTTGTCGACGACG | |
| | gagaccgccgtcgtcgacaagccgg | | GCGGTCTCCGTCGTCAGG | |
| | ccccgttgtcgacga | | ATCATCCGGgggccgtcttccc | |
| 19 | gCTCGTCGTCGACAACG | 37 | gTCTTCCCCTCCATCGTGG | 38 |
| | GCTCgttttagagctagaaatagca | | GGgttttagagctagaaatagcaagttaa | |
| | agttaaaataaggctagtccgttatcaa | | aataaggctagtccgttatcaacttgaaaa | |
| | cttgaaaaagtggcaccGAGTCG | | agtggcaccGAGTCGGTGCGG | |
| | GTGCccggatgatcctgacgacg | | CCGGCTTGTCGACGACGG | |
| | gagaccgccgtcgtcgacaagccgg | | CGGTCTCCGTCGTCAGGA | |
| | ccccgttgtcgacga | | TCATCCGGcacgatggagggg | |
| 20 | gCTCGTCGTCGACAACG | 39 | gCTGGGGCGCCCCACGAT | 40 |
| | GCTCgttttagagctagaaatagca | | GGAgttttagagctagaaatagcaagtt | |
| | agttaaaataaggctagtccgttatcaa | | aaaataaggctagtccgttatcaacttgaa | |
| | cttgaaaaagtggcaccGAGTCG | | aaagtggcaccGAGTCGGTGCG | |
| | GTGCccggatgatcctgacgacg | | GCCGGCTTGTCGACGACG | |
| | gagaccgccgtcgtcgacaagccgg | | GCGGTCTCCGTCGTCAGG | |
| | ccccgttgtcgacga | | ATCATCCGGatcgtggggcgcc | |
| 21 | gACCTCGGCTCACAGCG | 41 | GGCATCGTCGCCCGCGAA | 42 |
| | CGCCgttttagagctagaaatagca | | GCgttttagagctagaaatagcaagttaa | |
| | agttaaaataaggctagtccgttatcaa | | aataaggctagtccgttatcaacttgaaaa | |
| | cttgaaaaagtggcaccGAGTCG | | agtggcaccGAGTCGGTGCAC | |
| | GTGCacggagaccgccgtcgtcg | | GGCGGTCTCCGTCGTCAG | |
| | acaagccggccgcgctgtgagccg | | GATCATCCGGtcgcgggcgacg | |
| | | | a | |
| 22 | gACCTCGGCTCACAGCG | 43 | GGAGGGGAAGACGGCCC | 44 |
| | CGCCgttttagagctagaaatagca | | GGGgttttagagctagaaatagcaagtt | |
| | agttaaaataaggctagtccgttatcaa | | aaaataaggctagtccgttatcaacttgaa | |
| | cttgaaaaagtggcaccGAGTCG | | aaagtggcaccGAGTCGGTGCA | |
| | GTGCacggagaccgccgtcgtcg | | CGGCGGTCTCCGTCGTCA | |
| | acaagccggccgcgctgtgagccg | | GGATCATCCGGgggccgtcttc | |
| | | | cc | |
| 23 | gACCTCGGCTCACAGCG | 45 | gTCTTCCCCTCCATCGTGG | 46 |
| | CGCCgttttagagctagaaatagca | | GGgttttagagctagaaatagcaagttaa | |
| | agttaaaataaggctagtccgttatcaa | | aataaggctagtccgttatcaacttgaaaa | |
| | cttgaaaaagtggcaccGAGTCG | | agtggcaccGAGTCGGTGCAC | |
| | GTGCacggagaccgccgtcgtcg | | GGCGGTCTCCGTCGTCAG | |
| | acaagccggccgcgctgtgagccg | | GATCATCCGGcacgatggaggg | |
| | | | g | |
| 24 | gACCTCGGCTCACAGCG | 47 | gCTGGGGCGCCCCACGAT | 48 |
| | CGCCgttttagagctagaaatagca | | GGAgttttagagctagaaatagcaagtt | |
| | agttaaaataaggctagtccgttatcaa | | aaaataaggctagtccgttatcaacttgaa | |
| | cttgaaaaagtggcaccGAGTCG | | aaagtggcaccGAGTCGGTGCA | |
| | GTGCacggagaccgccgtcgtcg | | CGGCGGTCTCCGTCGTCA | |
| | acaagccggccgcgctgtgagccg | | GGATCATCCGGatcgtggggcg | |
| | | | cc | |
| 25 | GCTATTCTCGCAGCTCA | 49 | gCGGTAGTGACGCGTATT | 50 |
| | CCAgttttagagctagaaatagcaa | | GCCgttttagagctagaaatagcaagtt | |
| | gttaaaataaggctagtccgttatcaac | | aaaataaggctagtccgttatcaacttgaa | |
| | ttgaaaaagtggcaccGAGTCG | | aaagtggcaccGAGTCGGTGCc | |
| | GTGCacggagaccgccgtcgtcg | | cggatgatcctgacgacggagaccgccg | |
| | acaagccggcctgagctgcgagaa | | tcgtcgacaagccggccaatacgcgtca | |
| | | | ct | |
| 26 | GCTATTCTCGCAGCTCA | 51 | GGCATCGTCGCCCGCGAA | 52 |
| | CCAgttttagagctagaaatagcaa | | GCgttttagagctagaaatagcaagttaa | |
| | gttaaaataaggctagtccgttatcaac | | aataaggctagtccgttatcaacttgaaaa | |
| | ttgaaaaagtggcaccGAGTCG | | agtggcaccGAGTCGGTGCAC | |
| | GTGCacggagaccgccgtcgtcg | | GGCGGTCTCCGTCGTCAG | |
| | acaagccggcctgagctgcgagaa | | GATCATCCGGtcgcgggcgacg | |
| | | | a | |
| 27 | GCTATTCTCGCAGCTCA | 53 | GGAGGGGAAGACGGCCC | 54 |
| | CCAgttttagagctagaaatagcaa | | GGGgttttagagctagaaatagcaagtt | |
| | gttaaaataaggctagtccgttatcaac | | aaaataaggctagtccgttatcaacttgaa | |
| | ttgaaaaagtggcaccGAGTCG | | aaagtggcaccGAGTCGGTGCA | |
| | GTGCacggagaccgccgtcgtcg | | CGGCGGTCTCCGTCGTCA | |
| | acaagccggcctgagctgcgagaa | | GGATCATCCGGgggccgtcttc | |
| | | | cc | |
| 28 | GCTATTCTCGCAGCTCA | 55 | gTCTTCCCCTCCATCGTGG | 56 |
| | CCAgttttagagctagaaatagcaa | | GGgttttagagctagaaatagcaagttaa | |
| | gttaaaataaggctagtccgttatcaac | | aataaggctagtccgttatcaacttgaaaa | |
| | ttgaaaaagtggcaccGAGTCG | | agtggcaccGAGTCGGTGCAC | |
| | GTGCacggagaccgccgtcgtcg | | GGCGGTCTCCGTCGTCAG | |
| | acaagccggcctgagctgcgagaa | | GATCATCCGGcacgatggaggg | |
| | | | g | |
| 29 | GCCGCGCTCGTCGTCG | 57 | gCTGGGGCGCCCCACGAT | 58 |
| | ACAAgttttagagctagaaatagca | | GGAgttttagagctagaaatagcaagtt | |
| | agttaaaataaggctagtccgttatcaa | | aaaataaggctagtccgttatcaacttgaa | |
| | cttgaaaaagtggcaccGAGTCG | | aaagtggcaccGAGTCGGTGCA | |
| | GTGCacggagaccgccgtcgtcg | | CGGCGGTCTCCGTCGTCA | |
| | acaagccggcctcgacgacgagcg | | GGATCATCCGGatcgtggggcg | |
| | | | cc | |
| 30 | GCCGCGCTCGTCGTCG | 59 | gCGGTAGTGACGCGTATT | 60 |
| | ACAAgttttagagctagaaatagca | | GCCgttttagagctagaaatagcaagtt | |
| | agttaaaataaggctagtccgttatcaa | | aaaataaggctagtccgttatcaacttgaa | |
| | cttgaaaaagtggcaccGAGTCG | | aaagtggcaccGAGTCGGTGCc | |
| | GTGCacggagaccgccgtcgtcg | | cggatgatcctgacgacggagaccgccg | |
| | acaagccggcctcgacgacgagcg | | tcgtcgacaagccggccaatacgcgtca | |
| | | | ct | |
| 31 | GCCGCGCTCGTCGTCG | 61 | GGCATCGTCGCCCGCGAA | 62 |
| | ACAAgttttagagctagaaatagca | | GCgttttagagctagaaatagcaagttaa | |
| | agttaaaataaggctagtccgttatcaa | | aataaggctagtccgttatcaacttgaaaa | |
| | cttgaaaaagtggcaccGAGTCG | | agtggcaccGAGTCGGTGCAC | |
| | GTGCacggagaccgccgtcgtcg | | GGCGGTCTCCGTCGTCAG | |
| | acaagccggcctcgacgacgagcg | | GATCATCCGGtcgcgggcgacg | |
| | | | a | |
| 32 | GCCGCGCTCGTCGTCG | 63 | GGAGGGGAAGACGGCCC | 64 |
| | ACAAgttttagagctagaaatagca | | GGGgttttagagctagaaatagcaagtt | |
| | agttaaaataaggctagtccgttatcaa | | aaaataaggctagtccgttatcaacttgaa | |
| | cttgaaaaagtggcaccGAGTCG | | aaagtggcaccGAGTCGGTGCA | |
| | GTGCacggagaccgccgtcgtcg | | CGGCGGTCTCCGTCGTCA | |
| | acaagccggcctcgacgacgagcg | | GGATCATCCGGgggccgtcttc | |
| | | | cc | |
| 33 | gCTCGTCGTCGACAACG | 65 | gTCTTCCCCTCCATCGTGG | 66 |
| | GCTCgttttagagctagaaatagca | | GGgttttagagctagaaatagcaagttaa | |
| | agttaaaataaggctagtccgttatcaa | | aataaggctagtccgttatcaacttgaaaa | |
| | cttgaaaaagtggcaccGAGTCG | | agtggcaccGAGTCGGTGCAC | |
| | GTGCacggagaccgccgtcgtcg | | GGCGGTCTCCGTCGTCAG | |
| | acaagccggccccgttgtcgacga | | GATCATCCGGcacgatggaggg | |
| | | | g | |
| 34 | gCTCGTCGTCGACAACG | 67 | gCTGGGGCGCCCCACGAT | 68 |
| | GCTCgttttagagctagaaatagca | | GGAgttttagagctagaaatagcaagtt | |
| | agttaaaataaggctagtccgttatcaa | | aaaataaggctagtccgttatcaacttgaa | |
| | cttgaaaaagtggcaccGAGTCG | | aaagtggcaccGAGTCGGTGCA | |
| | GTGCacggagaccgccgtcgtcg | | CGGCGGTCTCCGTCGTCA | |
| | acaagccggccccgttgtcgacga | | GGATCATCCGGatcgtggggcg | |
| | | | cc | |
| 35 | gCTCGTCGTCGACAACG | 69 | gCGGTAGTGACGCGTATT | 70 |
| | GCTCgttttagagctagaaatagca | | GCCgttttagagctagaaatagcaagtt | |
| | agttaaaataaggctagtccgttatcaa | | aaaataaggctagtccgttatcaacttgaa | |
| | cttgaaaaagtggcaccGAGTCG | | aaagtggcaccGAGTCGGTGCc | |
| | GTGCacggagaccgccgtcgtcg | | cggatgatcctgacgacggagaccgccg | |
| | acaagccggccccgttgtcgacga | | tcgtcgacaagccggccaatacgcgtca | |
| | | | ct | |
| 36 | gCTCGTCGTCGACAACG | 71 | GGCATCGTCGCCCGCGAA | 72 |
| | GCTCgttttagagctagaaatagca | | GCgttttagagctagaaatagcaagttaa | |
| | agttaaaataaggctagtccgttatcaa | | aataaggctagtccgttatcaacttgaaaa | |
| | cttgaaaaagtggcaccGAGTCG | | agtggcaccGAGTCGGTGCAC | |
| | GTGCacggagaccgccgtcgtcg | | GGCGGTCTCCGTCGTCAG | |
| | acaagccggccccgttgtcgacga | | GATCATCCGGtcgcgggcgacg | |
| | | | a | |
| 37 | GAAGCCGGCCTTGCAC | 73 | GGAGGGGAAGACGGCCC | 74 |
| | ATGCgttttagagctagaaatagca | | GGGgttttagagctagaaatagcaagtt | |
| | agttaaaataaggctagtccgttatcaa | | aaaataaggctagtccgttatcaacttgaa | |
| | cttgaaaaagtggcaccGAGTCG | | aaagtggcaccGAGTCGGTGCA | |
| | GTGCACGGCGGTCTCC | | CGGCGGTCTCCGTCGTCA | |
| | GTCGTCAGGATCATCC | | GGATCATCCGGgggccgtcttc | |
| | GGtgtgcaaggccgg | | cc | |
| 38 | GAAGCCGGCCTTGCAC | 75 | gTCTTCCCCTCCATCGTGG | 76 |
| | ATGCgttttagagctagaaatagca | | GGgttttagagctagaaatagcaagttaa | |
| | agttaaaataaggctagtccgttatcaa | | aataaggctagtccgttatcaacttgaaaa | |
| | cttgaaaaagtggcaccGAGTCG | | agtggcaccGAGTCGGTGCAC | |
| | GTGCACGGCGGTCTCC | | GGCGGTCTCCGTCGTCAG | |
| | GTCGTCAGGATCATCC | | GATCATCCGGcacgatggaggg | |
| | GGtgtgcaaggccgg | | g | |
| 39 | GAAGCCGGCCTTGCAC | 77 | gCTGGGGCGCCCCACGAT | 78 |
| | ATGCgttttagagctagaaatagca | | GGAgttttagagctagaaatagcaagtt | |
| | agttaaaataaggctagtccgttatcaa | | aaaataaggctagtccgttatcaacttgaa | |
| | cttgaaaaagtggcaccGAGTCG | | aaagtggcaccGAGTCGGTGCA | |
| | GTGCACGGCGGTCTCC | | CGGCGGTCTCCGTCGTCA | |
| | GTCGTCAGGATCATCC | | GGATCATCCGGatcgtggggcg | |
| | GGtgtgcaaggccgg | | cc | |
| 40 | GAAGCCGGCCTTGCAC | 79 | gCGGTAGTGACGCGTATT | 80 |
| | ATGCgttttagagctagaaatagca | | GCCgttttagagctagaaatagcaagtt | |
| | agttaaaataaggctagtccgttatcaa | | aaaataaggctagtccgttatcaacttgaa | |
| | cttgaaaaagtggcaccGAGTCG | | aaagtggcaccGAGTCGGTGCc | |
| | GTGCACGGCGGTCTCC | | cggatgatcctgacgacggagaccgccg | |
| | GTCGTCAGGATCATCC | | tcgtcgacaagccggccaatacgcgtca | |
| | GGtgtgcaaggccgg | | ct | |

### Material and Methods—DNMT1 Mouse Locus

Cell culture Hepal-6 cells (American Type Culture Collection (ATCC)-CRL32156) were cultured in Dulbecco's Modified Eagle Medium with high glucose, sodium pyruvate, and GlutaMAX (Thermo Fisher Scientific), additionally supplemented with 10% (v/v) fetal bovine serum (FBS) and 1× penicillin-streptomycin (Thermo Fisher Scientific).

Transfection. Cells were plated at 5-15K the day prior to transfection in a 96-well plate coated with poly-D-lysine (BD Biocoat). Hepal-6 cells were transfected with Lipofectamine 3000 (Thermo Fisher Scientific), according to manufacturer's specifications. For AttB insertion, 35.5 ng of each dual guide plasmid and 100 ng SpCas9-RT plasmid were delivered to each well.

Genomic DNA extraction and purification and quantitation. DNA was harvested from transfected cells by removal of media, resuspension in 50 μL of QuickExtract (Lucigen), and incubation at 65° C. for 15 min, 68° C. for 15 min, and 98° C. for 10 min. Target regions were PCR amplified with NEBNext High-Fidelity 2× PCR Master Mix (NEB) based on the manufacturer's protocol. Barcodes and adapters for Illumina sequencing were added in a subsequent PCR amplification. Amplicons were pooled and prepared for sequencing on a MiSeq (Illumina). Reads were demultiplexed and analyzed with appropriate pipelines.

### Results—DNMT1 Locus

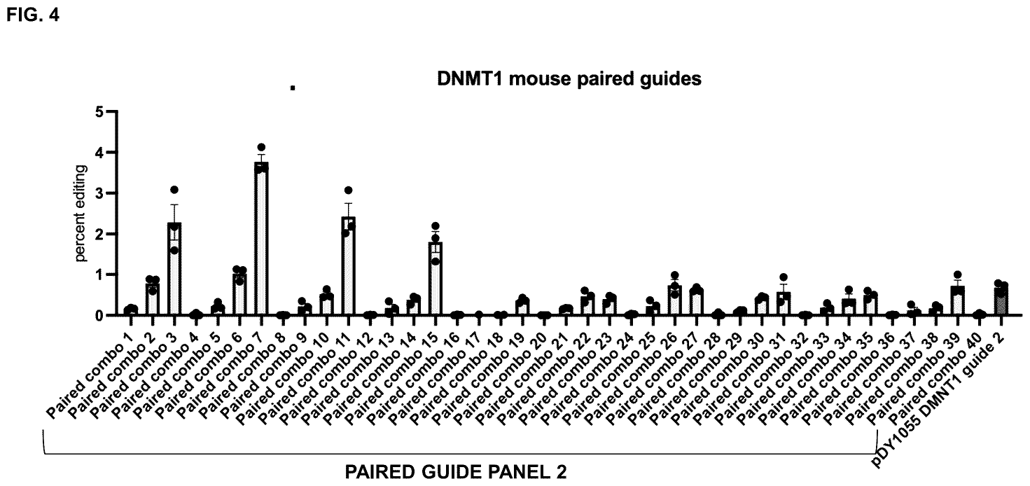

DNMT1 specific paired guides can yield higher levels of editing at mouse targets compared with Prime editing (FIG. 4). As such, paired guides can enable additional use of PASTE.

TABLE 2
Nucleic acid encoding Paired Guide Combinations for AttB insertion at the DNMT1
mouse locus

| | | SEQ | | SEQ |
|---|---|---|---|---|
| Pairing | Nucleic Acid Guide | ID | Nucleic Acid Guide | ID |
| Combo | Sequence 1 | NO | Sequence 2 | NO |
| 1 | gCGGGCTGGAGCTGTTCG | 81 | gCCGCGCGCGCGAAAAA | 82 |
| | CGCgttttagagctagaaatagcaagtt | | GCCGgttttagagctagaaatagcaa | |
| | aaaataaggctagtccgttatcaacttga | | gttaaaataaggctagtccgttatcaact | |
| | aaaagtggcaccGAGTCGGTGC | | tgaaaaagtggcaccGAGTCGG | |
| | GGCCGGCTTGTCGACGA | | TGCccggatgatcctgacgacggag | |
| | CGGCGGTCTCCGTCGTCA | | accgccgtcgtcgacaagccggccC | |
| | GGATCATCCGGCGAACA | | TTTTTCGCGCGC | |
| | GCTCCAG | | | |
| 2 | gCGGGCTGGAGCTGTTCG | 83 | gTTCCGCGCGCGCGAAA | 84 |
| | CGCgttttagagctagaaatagcaagtt | | AAGCgttttagagctagaaatagca | |
| | aaaataaggctagtccgttatcaacttga | | agttaaaataaggctagtccgttatcaac | |
| | aaaagtggcaccGAGTCGGTGC | | ttgaaaaagtggcaccGAGTCGG | |
| | GGCCGGCTTGTCGACGA | | TGCccggatgatcctgacgacggag | |
| | CGGCGGTCTCCGTCGTCA | | accgccgtcgtcgacaagccggccT | |
| | GGATCATCCGGCGAACA | | TTTCGCGCGCGC | |
| | GCTCCAG | | | |
| 3 | gCGGGCTGGAGCTGTTCG | 85 | gTTGCGCCGCCCCCTCCC | 86 |
| | CGCgttttagagctagaaatagcaagtt | | AATgttttagagctagaaatagcaag | |
| | aaaataaggctagtccgttatcaacttga | | ttaaaataaggctagtccgttatcaactt | |
| | aaaagtggcaccGAGTCGGTGC | | gaaaaagtggcaccGAGTCGGT | |
| | GGCCGGCTTGTCGACGA | | GCccggatgatcctgacgacggaga | |
| | CGGCGGTCTCCGTCGTCA | | ccgccgtcgtcgacaagccggccGG | |
| | GGATCATCCGGCGAACA | | GAGGGGGCGGC | |
| | GCTCCAG | | | |
| 4 | gCGGGCTGGAGCTGTTCG | 87 | gCCCCACTCTCTTGCCCT | 88 |
| | CGCgttttagagctagaaatagcaagtt | | GTGgttttagagctagaaatagcaag | |
| | aaaataaggctagtccgttatcaacttga | | ttaaaataaggctagtccgttatcaactt | |
| | aaaagtggcaccGAGTCGGTGC | | gaaaaagtggcaccGAGTCGGT | |
| | GGCCGGCTTGTCGACGA | | GCccggatgatcctgacgacggaga | |
| | CGGCGGTCTCCGTCGTCA | | ccgccgtcgtcgacaagccggccAG | |
| | GGATCATCCGGCGAACA | | GGCAAGAGAGT | |
| | GCTCCAG | | | |
| 5 | GGGAGGCAAGCGCAGGC | 89 | gCCGCGCGCGCGAAAAA | 90 |
| | ACTgttttagagctagaaatagcaagtt | | GCCGgttttagagctagaaatagcaa | |
| | aaaataaggctagtccgttatcaacttga | | gttaaaataaggctagtccgttatcaact | |
| | aaaagtggcaccGAGTCGGTGC | | tgaaaaagtggcaccGAGTCGG | |
| | GGCCGGCTTGTCGACGA | | TGCccggatgatcctgacgacggag | |
| | CGGCGGTCTCCGTCGTCA | | accgccgtcgtcgacaagccggccC | |
| | GGATCATCCGGGCCTGC | | TTTTTCGCGCGC | |
| | GCTTGCC | | | |
| 6 | GGGAGGCAAGCGCAGGC | 91 | gTTCCGCGCGCGCGAAA | 92 |
| | ACTgttttagagctagaaatagcaagtt | | AAGCgttttagagctagaaatagca | |
| | aaaataaggctagtccgttatcaacttga | | agttaaaataaggctagtccgttatcaac | |
| | aaaagtggcaccGAGTCGGTGC | | ttgaaaaagtggcaccGAGTCGG | |
| | GGCCGGCTTGTCGACGA | | TGCccggatgatcctgacgacggag | |
| | CGGCGGTCTCCGTCGTCA | | accgccgtcgtcgacaagccggccT | |
| | GGATCATCCGGGCCTGC | | TTTCGCGCGCGC | |
| | GCTTGCC | | | |
| 7 | GGGAGGCAAGCGCAGGC | 93 | gTTGCGCCGCCCCCTCCC | 94 |
| | ACTgttttagagctagaaatagcaagtt | | AATgttttagagctagaaatagcaag | |
| | aaaataaggctagtccgttatcaacttga | | ttaaaataaggctagtccgttatcaactt | |
| | aaaagtggcaccGAGTCGGTGC | | gaaaaagtggcaccGAGTCGGT | |
| | GGCCGGCTTGTCGACGA | | GCccggatgatcctgacgacggaga | |
| | CGGCGGTCTCCGTCGTCA | | ccgccgtcgtcgacaagccggccGG | |
| | GGATCATCCGGGCCTGC | | GAGGGGGCGGC | |
| | GCTTGCC | | | |
| 8 | GGGAGGCAAGCGCAGGC | 95 | gCCCCACTCTCTTGCCCT | 96 |
| | ACTgttttagagctagaaatagcaagtt | | GTGgttttagagctagaaatagcaag | |
| | aaaataaggctagtccgttatcaacttga | | ttaaaataaggctagtccgttatcaactt | |
| | aaaagtggcaccGAGTCGGTGC | | gaaaaagtggcaccGAGTCGGT | |
| | GGCCGGCTTGTCGACGA | | GCccggatgatcctgacgacggaga | |
| | CGGCGGTCTCCGTCGTCA | | ccgccgtcgtcgacaagccggccAG | |
| | GGATCATCCGGGCCTGC | | GGCAAGAGAGT | |
| | GCTTGCC | | | |
| 9 | GTCCGGGAGCGAGCCTG | 97 | gCCGCGCGCGCGAAAAA | 98 |
| | CCGgttttagagctagaaatagcaagtt | | GCCGgttttagagctagaaatagcaa | |
| | aaaataaggctagtccgttatcaacttga | | gttaaaataaggctagtccgttatcaact | |
| | aaaagtggcaccGAGTCGGTGC | | tgaaaaagtggcaccGAGTCGG | |
| | GGCCGGCTTGTCGACGA | | TGCccggatgatcctgacgacggag | |
| | CGGCGGTCTCCGTCGTCA | | accgccgtcgtcgacaagccggccC | |
| | GGATCATCCGGCAGGCT | | TTTTTCGCGCGC | |
| | CGCTCCC | | | |
| 10 | GTCCGGGAGCGAGCCTG | 99 | gTTCCGCGCGCGCGAAA | 100 |
| | CCGgttttagagctagaaatagcaagtt | | AAGCgttttagagctagaaatagca | |
| | aaaataaggctagtccgttatcaacttga | | agttaaaataaggctagtccgttatcaac | |
| | aaaagtggcaccGAGTCGGTGC | | ttgaaaaagtggcaccGAGTCGG | |
| | GGCCGGCTTGTCGACGA | | TGCccggatgatcctgacgacggag | |
| | CGGCGGTCTCCGTCGTCA | | accgccgtcgtcgacaagccggccT | |
| | GGATCATCCGGCAGGCT | | TTTCGCGCGCGC | |
| | CGCTCCC | | | |
| 11 | GTCCGGGAGCGAGCCTG | 101 | gTTGCGCCGCCCCCTCCC | 102 |
| | CCGgttttagagctagaaatagcaagtt | | AATgttttagagctagaaatagcaag | |
| | aaaataaggctagtccgttatcaacttga | | ttaaaataaggctagtccgttatcaactt | |
| | aaaagtggcaccGAGTCGGTGC | | gaaaaagtggcaccGAGTCGGT | |
| | GGCCGGCTTGTCGACGA | | GCccggatgatcctgacgacggaga | |
| | CGGCGGTCTCCGTCGTCA | | ccgccgtcgtcgacaagccggccGG | |
| | GGATCATCCGGCAGGCT | | GAGGGGGCGGC | |
| | CGCTCCC | | | |
| 12 | GTCCGGGAGCGAGCCTG | 103 | gCCCCACTCTCTTGCCCT | 104 |
| | CCGgttttagagctagaaatagcaagtt | | GTGgttttagagctagaaatagcaag | |
| | aaaataaggctagtccgttatcaacttga | | ttaaaataaggctagtccgttatcaactt | |
| | aaaagtggcaccGAGTCGGTGC | | gaaaaagtggcaccGAGTCGGT | |
| | GGCCGGCTTGTCGACGA | | GCccggatgatcctgacgacggaga | |
| | CGGCGGTCTCCGTCGTCA | | ccgccgtcgtcgacaagccggccAG | |
| | GGATCATCCGGCAGGCT | | GGCAAGAGAGT | |
| | CGCTCCC | | | |
| 13 | gTGTTCGCGCTGGCATCT | 105 | gCCGCGCGCGCGAAAAA | 106 |
| | TGCgttttagagctagaaatagcaagtt | | GCCGgttttagagctagaaatagcaa | |
| | aaaataaggctagtccgttatcaacttga | | gttaaaataaggctagtccgttatcaact | |
| | aaaagtggcaccGAGTCGGTGC | | tgaaaaagtggcaccGAGTCGG | |
| | GGCCGGCTTGTCGACGA | | TGCccggatgatcctgacgacggag | |
| | CGGCGGTCTCCGTCGTCA | | accgccgtcgtcgacaagccggccC | |
| | GGATCATCCGGAGATGC | | TTTTTCGCGCGC | |
| | CAGCGCG | | | |
| 14 | gTGTTCGCGCTGGCATCT | 107 | gTTCCGCGCGCGCGAAA | 108 |
| | TGCgttttagagctagaaatagcaagtt | | AAGCgttttagagctagaaatagca | |
| | aaaataaggctagtccgttatcaacttga | | agttaaaataaggctagtccgttatcaac | |
| | aaaagtggcaccGAGTCGGTGC | | ttgaaaaagtggcaccGAGTCGG | |
| | GGCCGGCTTGTCGACGA | | TGCccggatgatcctgacgacggag | |
| | CGGCGGTCTCCGTCGTCA | | accgccgtcgtcgacaagccggccT | |
| | GGATCATCCGGAGATGC | | TTTCGCGCGCGC | |
| | CAGCGCG | | | |
| 15 | gTGTTCGCGCTGGCATCT | 109 | gTTGCGCCGCCCCCTCCC | 110 |
| | TGCgttttagagctagaaatagcaagtt | | AATgttttagagctagaaatagcaag | |
| | aaaataaggctagtccgttatcaacttga | | ttaaaataaggctagtccgttatcaactt | |
| | aaaagtggcaccGAGTCGGTGC | | gaaaaagtggcaccGAGTCGGT | |
| | GGCCGGCTTGTCGACGA | | GCccggatgatcctgacgacggaga | |
| | CGGCGGTCTCCGTCGTCA | | ccgccgtcgtcgacaagccggccGG | |
| | GGATCATCCGGAGATGC | | GAGGGGGCGGC | |
| | CAGCGCG | | | |
| 16 | gTGTTCGCGCTGGCATCT | 111 | gCCCCACTCTCTTGCCCT | 112 |
| | TGCgttttagagctagaaatagcaagtt | | GTGgttttagagctagaaatagcaag | |
| | aaaataaggctagtccgttatcaacttga | | ttaaaataaggctagtccgttatcaactt | |
| | aaaagtggcaccGAGTCGGTGC | | gaaaaagtggcaccGAGTCGGT | |
| | GGCCGGCTTGTCGACGA | | GCccggatgatcctgacgacggaga | |
| | CGGCGGTCTCCGTCGTCA | | ccgccgtcgtcgacaagccggccAG | |
| | GGATCATCCGGAGATGC | | GGCAAGAGAGT | |
| | CAGCGCG | | | |
| 17 | gAACAGCTCTGAACGAG | 113 | gCCGCGCGCGCGAAAAA | 114 |
| | ACCCgttttagagctagaaatagcaa | | GCCGgttttagagctagaaatagcaa | |
| | gttaaaataaggctagtccgttatcaactt | | gttaaaataaggctagtccgttatcaact | |
| | gaaaaagtggcaccGAGTCGGT | | tgaaaaagtggcaccGAGTCGG | |
| | GCGGCCGGCTTGTCGAC | | TGCccggatgatcctgacgacggag | |
| | GACGGCGGTCTCCGTCGT | | accgccgtcgtcgacaagccggccC | |
| | CAGGATCATCCGGTCTCG | | TTTTTCGCGCGC | |
| | TTCAGAGC | | | |
| 18 | gAACAGCTCTGAACGAG | 115 | gTTCCGCGCGCGCGAAA | 116 |
| | ACCCgttttagagctagaaatagcaa | | AAGCgttttagagctagaaatagca | |
| | gttaaaataaggctagtccgttatcaactt | | agttaaaataaggctagtccgttatcaac | |
| | gaaaaagtggcaccGAGTCGGT | | ttgaaaaagtggcaccGAGTCGG | |
| | GCGGCCGGCTTGTCGAC | | TGCccggatgatcctgacgacggag | |
| | GACGGCGGTCTCCGTCGT | | accgccgtcgtcgacaagccggccT | |
| | CAGGATCATCCGGTCTCG | | TTTCGCGCGCGC | |
| | TTCAGAGC | | | |
| 19 | gAACAGCTCTGAACGAG | 117 | gTTGCGCCGCCCCCTCCC | 118 |
| | ACCCgttttagagctagaaatagcaa | | AATgttttagagctagaaatagcaag | |
| | gttaaaataaggctagtccgttatcaactt | | ttaaaataaggctagtccgttatcaactt | |
| | gaaaaagtggcaccGAGTCGGT | | gaaaaagtggcaccGAGTCGGT | |
| | GCGGCCGGCTTGTCGAC | | GCccggatgatcctgacgacggaga | |
| | GACGGCGGTCTCCGTCGT | | ccgccgtcgtcgacaagccggccGG | |
| | CAGGATCATCCGGTCTCG | | GAGGGGGCGGC | |
| | TTCAGAGC | | | |
| 20 | gAACAGCTCTGAACGAG | 119 | gCCCCACTCTCTTGCCCT | 120 |
| | ACCCgttttagagctagaaatagcaa | | GTGgttttagagctagaaatagcaag | |
| | gttaaaataaggctagtccgttatcaactt | | ttaaaataaggctagtccgttatcaactt | |
| | gaaaaagtggcaccGAGTCGGT | | gaaaaagtggcaccGAGTCGGT | |
| | GCGGCCGGCTTGTCGAC | | GCccggatgatcctgacgacggaga | |
| | GACGGCGGTCTCCGTCGT | | ccgccgtcgtcgacaagccggccAG | |
| | CAGGATCATCCGGTCTCG | | GGCAAGAGAGT | |
| | TTCAGAGC | | | |
| 21 | gCGGGCTGGAGCTGTTCG | 121 | gCCGCGCGCGCGAAAAA | 122 |
| | CGCgttttagagctagaaatagcaagtt | | GCCGgttttagagctagaaatagcaa | |
| | aaaataaggctagtccgttatcaacttga | | gttaaaataaggctagtccgttatcaact | |
| | aaaagtggcaccGAGTCGGTGC | | tgaaaaagtggcaccGAGTCGG | |
| | ACGGCGGTCTCCGTCGTC | | TGCacggagaccgccgtcgtcgaca | |
| | AGGATCATCCGGCGAAC | | agccggccCTTTTTCGCGCG | |
| | AGCTCCAG | | C | |
| 22 | gCGGGCTGGAGCTGTTCG | 123 | gTTCCGCGCGCGCGAAA | 124 |
| | CGCgttttagagctagaaatagcaagtt | | AAGCgttttagagctagaaatagca | |
| | aaaataaggctagtccgttatcaacttga | | agttaaaataaggctagtccgttatcaac | |
| | aaaagtggcaccGAGTCGGTGC | | ttgaaaaagtggcaccGAGTCGG | |
| | ACGGCGGTCTCCGTCGTC | | TGCacggagaccgccgtcgtcgaca | |
| | AGGATCATCCGGCGAAC | | agccggccTTTTCGCGCGCG | |
| | AGCTCCAG | | C | |
| 23 | gCGGGCTGGAGCTGTTCG | 125 | gTTGCGCCGCCCCCTCCC | 126 |
| | CGCgttttagagctagaaatagcaagtt | | AATgttttagagctagaaatagcaag | |
| | aaaataaggctagtccgttatcaacttga | | ttaaaataaggctagtccgttatcaactt | |
| | aaaagtggcaccGAGTCGGTGC | | gaaaaagtggcaccGAGTCGGT | |
| | ACGGCGGTCTCCGTCGTC | | GCacggagaccgccgtcgtcgacaa | |
| | AGGATCATCCGGCGAAC | | gccggccGGGAGGGGGCG | |
| | AGCTCCAG | | GC | |
| 24 | gCGGGCTGGAGCTGTTCG | 127 | gCCCCACTCTCTTGCCCT | 128 |
| | CGCgttttagagctagaaatagcaagtt | | GTGgttttagagctagaaatagcaag | |
| | aaaataaggctagtccgttatcaacttga | | ttaaaataaggctagtccgttatcaactt | |
| | aaaagtggcaccGAGTCGGTGC | | gaaaaagtggcaccGAGTCGGT | |
| | ACGGCGGTCTCCGTCGTC | | GCacggagaccgccgtcgtcgacaa | |
| | AGGATCATCCGGCGAAC | | gccggccAGGGCAAGAGA | |
| | AGCTCCAG | | GT | |
| 25 | GGGAGGCAAGCGCAGGC | 129 | gCCGCGCGCGCGAAAAA | 130 |
| | ACTgttttagagctagaaatagcaagtt | | GCCGgttttagagctagaaatagcaa | |
| | aaaataaggctagtccgttatcaacttga | | gttaaaataaggctagtccgttatcaact | |
| | aaaagtggcaccGAGTCGGTGC | | tgaaaaagtggcaccGAGTCGG | |
| | ACGGCGGTCTCCGTCGTC | | TGCacggagaccgccgtcgtcgaca | |
| | AGGATCATCCGGGCCTG | | agccggccCTTTTTCGCGCG | |
| | CGCTTGCC | | C | |
| 26 | GGGAGGCAAGCGCAGGC | 131 | gTTCCGCGCGCGCGAAA | 132 |
| | ACTgttttagagctagaaatagcaagtt | | AAGCgttttagagctagaaatagca | |
| | aaaataaggctagtccgttatcaacttga | | agttaaaataaggctagtccgttatcaac | |
| | aaaagtggcaccGAGTCGGTGC | | ttgaaaaagtggcaccGAGTCGG | |
| | ACGGCGGTCTCCGTCGTC | | TGCacggagaccgccgtcgtcgaca | |
| | AGGATCATCCGGGCCTG | | agccggccTTTTCGCGCGCG | |
| | CGCTTGCC | | C | |
| 27 | GGGAGGCAAGCGCAGGC | 133 | gTTGCGCCGCCCCCTCCC | 134 |
| | ACTgttttagagctagaaatagcaagtt | | AATgttttagagctagaaatagcaag | |
| | aaaataaggctagtccgttatcaacttga | | ttaaaataaggctagtccgttatcaactt | |
| | aaaagtggcaccGAGTCGGTGC | | gaaaaagtggcaccGAGTCGGT | |
| | ACGGCGGTCTCCGTCGTC | | GCacggagaccgccgtcgtcgacaa | |
| | AGGATCATCCGGGCCTG | | gccggccGGGAGGGGGCG | |
| | CGCTTGCC | | GC | |
| 28 | GGGAGGCAAGCGCAGGC | 135 | gCCCCACTCTCTTGCCCT | 136 |
| | ACTgttttagagctagaaatagcaagtt | | GTGgttttagagctagaaatagcaag | |
| | aaaataaggctagtccgttatcaacttga | | ttaaaataaggctagtccgttatcaactt | |
| | aaaagtggcaccGAGTCGGTGC | | gaaaaagtggcaccGAGTCGGT | |
| | ACGGCGGTCTCCGTCGTC | | GCacggagaccgccgtcgtcgacaa | |
| | AGGATCATCCGGGCCTG | | gccggccAGGGCAAGAGA | |
| | CGCTTGCC | | GT | |
| 29 | GTCCGGGAGCGAGCCTG | 137 | gCCGCGCGCGCGAAAAA | 138 |
| | CCGgttttagagctagaaatagcaagtt | | GCCGgttttagagctagaaatagcaa | |
| | aaaataaggctagtccgttatcaacttga | | gttaaaataaggctagtccgttatcaact | |
| | aaaagtggcaccGAGTCGGTGC | | tgaaaaagtggcaccGAGTCGG | |
| | ACGGCGGTCTCCGTCGTC | | TGCacggagaccgccgtcgtcgaca | |
| | AGGATCATCCGGCAGGC | | agccggccCTTTTTCGCGCG | |
| | TCGCTCCC | | C | |
| 30 | GTCCGGGAGCGAGCCTG | 139 | gTTCCGCGCGCGCGAAA | 140 |
| | CCGgttttagagctagaaatagcaagtt | | AAGCgttttagagctagaaatagca | |
| | aaaataaggctagtccgttatcaacttga | | agttaaaataaggctagtccgttatcaac | |
| | aaaagtggcaccGAGTCGGTGC | | ttgaaaaagtggcaccGAGTCGG | |
| | ACGGCGGTCTCCGTCGTC | | TGCacggagaccgccgtcgtcgaca | |
| | AGGATCATCCGGCAGGC | | agccggccTTTTCGCGCGCG | |
| | TCGCTCCC | | C | |
| 31 | GTCCGGGAGCGAGCCTG | 141 | gTTGCGCCGCCCCCTCCC | 142 |
| | CCGgttttagagctagaaatagcaagtt | | AATgttttagagctagaaatagcaag | |
| | aaaataaggctagtccgttatcaacttga | | ttaaaataaggctagtccgttatcaactt | |
| | aaaagtggcaccGAGTCGGTGC | | gaaaaagtggcaccGAGTCGGT | |
| | ACGGCGGTCTCCGTCGTC | | GCacggagaccgccgtcgtcgacaa | |
| | AGGATCATCCGGCAGGC | | gccggccGGGAGGGGGCG | |
| | TCGCTCCC | | GC | |
| 32 | GTCCGGGAGCGAGCCTG | 143 | gCCCCACTCTCTTGCCCT | 144 |
| | CCGgttttagagctagaaatagcaagtt | | GTGgttttagagctagaaatagcaag | |
| | aaaataaggctagtccgttatcaacttga | | ttaaaataaggctagtccgttatcaactt | |
| | aaaagtggcaccGAGTCGGTGC | | gaaaaagtggcaccGAGTCGGT | |
| | ACGGCGGTCTCCGTCGTC | | GCacggagaccgccgtcgtcgacaa | |
| | AGGATCATCCGGCAGGC | | gccggccAGGGCAAGAGA | |
| | TCGCTCCC | | GT | |
| 33 | gTGTTCGCGCTGGCATCT | 145 | gCCGCGCGCGCGAAAAA | 146 |
| | TGCgttttagagctagaaatagcaagtt | | GCCGgttttagagctagaaatagcaa | |
| | aaaataaggctagtccgttatcaacttga | | gttaaaataaggctagtccgttatcaact | |
| | aaaagtggcaccGAGTCGGTGC | | tgaaaaagtggcaccGAGTCGG | |
| | ACGGCGGTCTCCGTCGTC | | TGCacggagaccgccgtcgtcgaca | |
| | AGGATCATCCGGAGATG | | agccggccCTTTTTCGCGCG | |
| | CCAGCGCG | | C | |
| 34 | gTGTTCGCGCTGGCATCT | 147 | gTTCCGCGCGCGCGAAA | 148 |
| | TGCgttttagagctagaaatagcaagtt | | AAGCgttttagagctagaaatagca | |
| | aaaataaggctagtccgttatcaacttga | | agttaaaataaggctagtccgttatcaac | |
| | aaaagtggcaccGAGTCGGTGC | | ttgaaaaagtggcaccGAGTCGG | |
| | ACGGCGGTCTCCGTCGTC | | TGCacggagaccgccgtcgtcgaca | |
| | AGGATCATCCGGAGATG | | agccggccTTTTCGCGCGCG | |
| | CCAGCGCG | | C | |
| 35 | gTGTTCGCGCTGGCATCT | 149 | gTTGCGCCGCCCCCTCCC | 150 |
| | TGCgttttagagctagaaatagcaagtt | | AATgttttagagctagaaatagcaag | |
| | aaaataaggctagtccgttatcaacttga | | ttaaaataaggctagtccgttatcaactt | |
| | aaaagtggcaccGAGTCGGTGC | | gaaaaagtggcaccGAGTCGGT | |
| | ACGGCGGTCTCCGTCGTC | | GCacggagaccgccgtcgtcgacaa | |
| | AGGATCATCCGGAGATG | | gccggccGGGAGGGGGCG | |
| | CCAGCGCG | | GC | |
| 36 | gTGTTCGCGCTGGCATCT | 151 | gCCCCACTCTCTTGCCCT | 152 |
| | TGCgttttagagctagaaatagcaagtt | | GTGgttttagagctagaaatagcaag | |
| | aaaataaggctagtccgttatcaacttga | | ttaaaataaggctagtccgttatcaactt | |
| | aaaagtggcaccGAGTCGGTGC | | gaaaaagtggcaccGAGTCGGT | |
| | ACGGCGGTCTCCGTCGTC | | GCacggagaccgccgtcgtcgacaa | |
| | AGGATCATCCGGAGATG | | gccggccAGGGCAAGAGA | |
| | CCAGCGCG | | GT | |
| 37 | gAACAGCTCTGAACGAG | 153 | gCCGCGCGCGCGAAAAA | 154 |
| | ACCCgttttagagctagaaatagcaa | | GCCGgttttagagctagaaatagcaa | |
| | gttaaaataaggctagtccgttatcaactt | | gttaaaataaggctagtccgttatcaact | |
| | gaaaaagtggcaccGAGTCGGT | | tgaaaaagtggcaccGAGTCGG | |
| | GCACGGCGGTCTCCGTC | | TGCacggagaccgccgtcgtcgaca | |
| | GTCAGGATCATCCGGTCT | | agccggccCTTTTTCGCGCG | |
| | CGTTCAGAGC | | C | |
| 38 | gAACAGCTCTGAACGAG | 155 | gTTCCGCGCGCGCGAAA | 156 |
| | ACCCgttttagagctagaaatagcaa | | AAGCgttttagagctagaaatagca | |
| | gttaaaataaggctagtccgttatcaactt | | agttaaaataaggctagtccgttatcaac | |
| | gaaaaagtggcaccGAGTCGGT | | ttgaaaaagtggcaccGAGTCGG | |
| | GCACGGCGGTCTCCGTC | | TGCacggagaccgccgtcgtcgaca | |
| | GTCAGGATCATCCGGTCT | | agccggccTTTTCGCGCGCG | |
| | CGTTCAGAGC | | C | |
| 39 | gAACAGCTCTGAACGAG | 157 | gTTGCGCCGCCCCCTCCC | 158 |
| | ACCCgttttagagctagaaatagcaa | | AATgttttagagctagaaatagcaag | |
| | gttaaaataaggctagtccgttatcaactt | | ttaaaataaggctagtccgttatcaactt | |
| | gaaaaagtggcaccGAGTCGGT | | gaaaaagtggcaccGAGTCGGT | |
| | GCACGGCGGTCTCCGTC | | GCacggagaccgccgtcgtcgacaa | |
| | GTCAGGATCATCCGGTCT | | gccggccGGGAGGGGGCG | |
| | CGTTCAGAGC | | GC | |
| 40 | gAACAGCTCTGAACGAG | 159 | gCCCCACTCTCTTGCCCT | 160 |
| | ACCCgttttagagctagaaatagcaa | | GTGgttttagagctagaaatagcaag | |
| | gttaaaataaggctagtccgttatcaactt | | ttaaaataaggctagtccgttatcaactt | |
| | gaaaaagtggcaccGAGTCGGT | | gaaaaagtggcaccGAGTCGGT | |
| | GCACGGCGGTCTCCGTC | | GCacggagaccgccgtcgtcgacaa | |
| | GTCAGGATCATCCGGTCT | | gccggccAGGGCAAGAGA | |
| | CGTTCAGAGC | | GT | |

### Material and Methods—NOLC Mouse Locus

Cell culture. Hepal -6 cells (American Type Culture Collection (ATCC)-CRL32156) were cultured in Dulbecco's Modified Eagle Medium with high glucose, sodium pyruvate, and GlutaMAX (Thermo Fisher Scientific), additionally supplemented with 10% (v/v) fetal bovine serum (FBS) and 1× penicillin-streptomycin (Thermo Fisher Scientific).

Transfection. Cells were plated at 5-15K the day prior to transfection in a 96-well plate coated with poly-D-lysine (BD Biocoat). Hepal-6 cells were transfected with Lipofectamine 3000 (Thermo Fisher Scientific), according to manufacturer's specifications. For AttB insertion, 35.5ng of each dual guide plasmid, and 100 ng SpCas9-RT plasmid were delivered to each well.

Genomic DNA extraction and purification and quantitation. DNA was harvested from transfected cells by removal of media, resuspension in 50 μL of QuickExtract (Lucigen), and incubation at 65° C. for 15 min, 68° C. for 15 min, and 98° C. for 10 min. Target regions were PCR amplified with NEBNext High-Fidelity 2× PCR Master Mix (NEB) based on the manufacturer's protocol. Barcodes and adapters for Illumina sequencing were added in a subsequent PCR amplification. Amplicons were pooled and prepared for sequencing on a MiSeq (Illumina). Reads were demultiplexed and analyzed with appropriate pipelines.

### Results—NOLC1 Mouse Locus

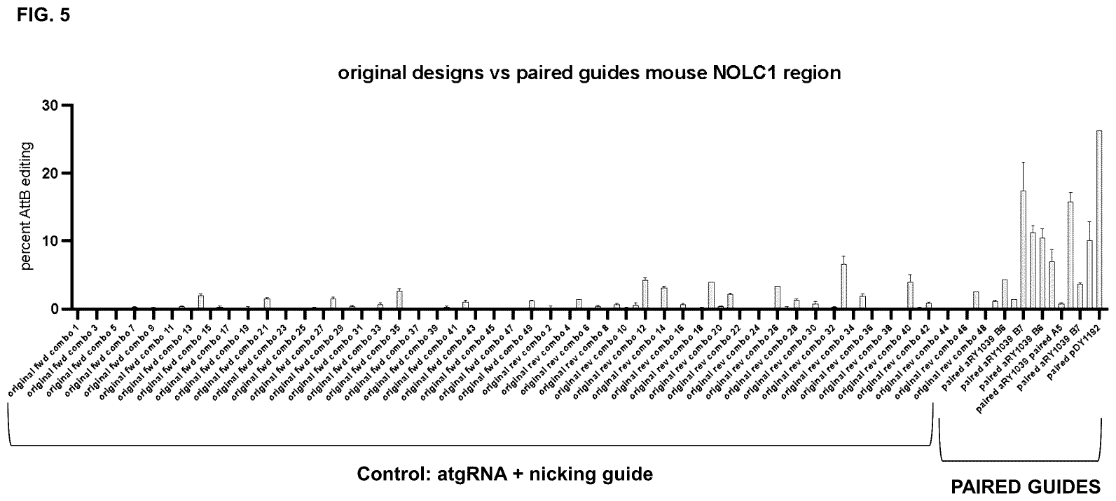

The amount of attb integration using paired guides outperforms the attb integration efficiency of most combinations of distinct single atgRNA plus nicking guide (FIG. 5).

TABLE 3
Nucleic acid encoding Paired Guide Combinations for AttB insertion at the NOLC
mouse locus

| | | SEQ | | SEQ |
|---|---|---|---|---|
| Pairing | Nucleic Acid Guide | ID | Nucleic Acid Guide | ID |
| Combo | Sequence 1 | NO | Sequence 2 | NO |
| 1 | gCTTGTCGGCTTTAGAAG | 161 | gCAGAGAAGCTGGGCAG | 162 |
| | TTAgttttagagctagaaatagcaagtt | | ACAAgttttagagctagaaatagca | |
| | aaaataaggctagtccgttatcaacttga | | agttaaaataaggctagtccgttatcaac | |
| | aaaagtggcaccGAGTCGGTGC | | ttgaaaaagtggcaccGAGTCGG | |
| | CCCTCGCCTCCTTAAATG | | TGC | |
| | ATCCTGACGACGGAGAC | | | |
| | CGCCGTCGTCGACAAGC | | | |
| | C | | | |
| 2 | GTCGGCTTTAGAAGTTAA | 163 | gCAGAGAAGCTGGGCAG | 164 |
| | GGgttttagagctagaaatagcaagtta | | ACAAgttttagagctagaaatagca | |
| | aaataaggctagtccgttatcaacttgaa | | agttaaaataaggctagtccgttatcaac | |
| | aaagtggcaccGAGTCGGTGC | | ttgaaaaagtggcaccGAGTCGG | |
| | CAGCCCTCGCCTCCTATG | | TGC | |
| | ATCCTGACGACGGAGAC | | | |
| | CGCCGTCGTCGACAAGC | | | |
| | C | | | |
| 3 | gCTTTAGAAGTTAAGGAG | 165 | gCAGAGAAGCTGGGCAG | 166 |
| | GCGgttttagagctagaaatagcaagtt | | ACAAgttttagagctagaaatagca | |
| | aaaataaggctagtccgttatcaacttga | | agttaaaataaggctagtccgttatcaac | |
| | aaaagtggcaccGAGTCGGTGC | | ttgaaaaagtggcaccGAGTCGG | |
| | GACCCCAGCCCTCGCAT | | TGC | |
| | GATCCTGACGACGGAGA | | | |
| | CCGCCGTCGTCGACAAG | | | |
| | CC | | | |
| 4 | gTTTAGAAGTTAAGGAGG | 167 | gCAGAGAAGCTGGGCAG | 168 |
| | CGAgttttagagctagaaatagcaagtt | | ACAAgttttagagctagaaatagca | |
| | aaaataaggctagtccgttatcaacttga | | agttaaaataaggctagtccgttatcaac | |
| | aaaagtggcaccGAGTCGGTGC | | ttgaaaaagtggcaccGAGTCGG | |
| | AGACCCCAGCCCTCGAT | | TGC | |
| | GATCCTGACGACGGAGA | | | |
| | CCGCCGTCGTCGACAAG | | | |
| | CC | | | |
| 5 | GAAGTTAAGGAGGCGAG | 169 | gCAGAGAAGCTGGGCAG | 170 |
| | GGCgttttagagctagaaatagcaagtt | | ACAAgttttagagctagaaatagca | |
| | aaaataaggctagtccgttatcaacttga | | agttaaaataaggctagtccgttatcaac | |
| | aaaagtggcaccGAGTCGGTGC | | ttgaaaaagtggcaccGAGTCGG | |
| | TGACAGACCCCAGCCAT | | TGC | |
| | GATCCTGACGACGGAGA | | | |
| | CCGCCGTCGTCGACAAG | | | |
| | CC | | | |
| 6 | gAAGTTAAGGAGGCGAG | 171 | gCAGAGAAGCTGGGCAG | 172 |
| | GGCTgttttagagctagaaatagcaa | | ACAAgttttagagctagaaatagca | |
| | gttaaaataaggctagtccgttatcaactt | | agttaaaataaggctagtccgttatcaac | |
| | gaaaaagtggcaccGAGTCGGT | | ttgaaaaagtggcaccGAGTCGG | |
| | GCCTGACAGACCCCAGC | | TGC | |
| | ATGATCCTGACGACGGA | | | |
| | GACCGCCGTCGTCGACA | | | |
| | AGCC | | | |
| 7 | gAGTTAAGGAGGCGAGG | 173 | gCAGAGAAGCTGGGCAG | 174 |
| | GCTGgttttagagctagaaatagcaa | | ACAAgttttagagctagaaatagca | |
| | gttaaaataaggctagtccgttatcaactt | | agttaaaataaggctagtccgttatcaac | |
| | gaaaaagtggcaccGAGTCGGT | | ttgaaaaagtggcaccGAGTCGG | |
| | GCACTGACAGACCCCAG | | TGC | |
| | ATGATCCTGACGACGGA | | | |
| | GACCGCCGTCGTCGACA | | | |
| | AGCC | | | |
| 8 | gCTTGTCGGCTTTAGAAG | 175 | GGAAGGTCCGCAGAGA | 176 |
| | TTAgttttagagctagaaatagcaagtt | | AGCTgttttagagctagaaatagcaa | |
| | aaaataaggctagtccgttatcaacttga | | gttaaaataaggctagtccgttatcaact | |
| | aaaagtggcaccGAGTCGGTGC | | tgaaaaagtggcaccGAGTCGG | |
| | CCCTCGCCTCCTTAAATG | | TGC | |
| | ATCCTGACGACGGAGAC | | | |
| | CGCCGTCGTCGACAAGC | | | |
| | C | | | |
| 9 | GTCGGCTTTAGAAGTTAA | 177 | GGAAGGTCCGCAGAGA | 178 |
| | GGgttttagagctagaaatagcaagtta | | AGCTgttttagagctagaaatagcaa | |
| | aaataaggctagtccgttatcaacttgaa | | gttaaaataaggctagtccgttatcaact | |
| | aaagtggcaccGAGTCGGTGC | | tgaaaaagtggcaccGAGTCGG | |
| | CAGCCCTCGCCTCCTATG | | TGC | |
| | ATCCTGACGACGGAGAC | | | |
| | CGCCGTCGTCGACAAGC | | | |
| | C | | | |
| 10 | gCTTTAGAAGTTAAGGAG | 179 | GGAAGGTCCGCAGAGA | 180 |
| | GCGgttttagagctagaaatagcaagtt | | AGCTgttttagagctagaaatagcaa | |
| | aaaataaggctagtccgttatcaacttga | | gttaaaataaggctagtccgttatcaact | |
| | aaaagtggcaccGAGTCGGTGC | | tgaaaaagtggcaccGAGTCGG | |
| | GACCCCAGCCCTCGCAT | | TGC | |
| | GATCCTGACGACGGAGA | | | |
| | CCGCCGTCGTCGACAAG | | | |
| | CC | | | |
| 11 | gTTTAGAAGTTAAGGAGG | 181 | GGAAGGTCCGCAGAGA | 182 |
| | CGAgttttagagctagaaatagcaagtt | | AGCTgttttagagctagaaatagcaa | |
| | aaaataaggctagtccgttatcaacttga | | gttaaaataaggctagtccgttatcaact | |
| | aaaagtggcaccGAGTCGGTGC | | tgaaaaagtggcaccGAGTCGG | |
| | AGACCCCAGCCCTCGAT | | TGC | |
| | GATCCTGACGACGGAGA | | | |
| | CCGCCGTCGTCGACAAG | | | |
| | CC | | | |
| 12 | GAAGTTAAGGAGGCGAG | 183 | GGAAGGTCCGCAGAGA | 184 |
| | GGCgttttagagctagaaatagcaagtt | | AGCTgttttagagctagaaatagcaa | |
| | aaaataaggctagtccgttatcaacttga | | gttaaaataaggctagtccgttatcaact | |
| | aaaagtggcaccGAGTCGGTGC | | tgaaaaagtggcaccGAGTCGG | |
| | TGACAGACCCCAGCCAT | | TGC | |
| | GATCCTGACGACGGAGA | | | |
| | CCGCCGTCGTCGACAAG | | | |
| | CC | | | |
| 13 | gAAGTTAAGGAGGCGAG | 185 | GGAAGGTCCGCAGAGA | 186 |
| | GGCTgttttagagctagaaatagcaa | | AGCTgttttagagctagaaatagcaa | |
| | gttaaaataaggctagtccgttatcaactt | | gttaaaataaggctagtccgttatcaact | |
| | gaaaaagtggcaccGAGTCGGT | | tgaaaaagtggcaccGAGTCGG | |
| | GCCTGACAGACCCCAGC | | TGC | |
| | ATGATCCTGACGACGGA | | | |
| | GACCGCCGTCGTCGACA | | | |
| | AGCC | | | |
| 14 | gAGTTAAGGAGGCGAGG | 187 | GGAAGGTCCGCAGAGA | 188 |
| | GCTGgttttagagctagaaatagcaa | | AGCTgttttagagctagaaatagcaa | |
| | gttaaaataaggctagtccgttatcaactt | | gttaaaataaggctagtccgttatcaact | |
| | gaaaaagtggcaccGAGTCGGT | | tgaaaaagtggcaccGAGTCGG | |
| | GCACTGACAGACCCCAG | | TGC | |
| | ATGATCCTGACGACGGA | | | |
| | GACCGCCGTCGTCGACA | | | |
| | AGCC | | | |
| 15 | gCTTGTCGGCTTTAGAAG | 189 | gAGGAAGGTCCGCAGAG | 190 |
| | TTAgttttagagctagaaatagcaagtt | | AAGCgttttagagctagaaatagca | |
| | aaaataaggctagtccgttatcaacttga | | agttaaaataaggctagtccgttatcaac | |
| | aaaagtggcaccGAGTCGGTGC | | ttgaaaaagtggcaccGAGTCGG | |
| | CCCTCGCCTCCTTAAATG | | TGC | |
| | ATCCTGACGACGGAGAC | | | |
| | CGCCGTCGTCGACAAGC | | | |
| | C | | | |
| 16 | GTCGGCTTTAGAAGTTAA | 191 | gAGGAAGGTCCGCAGAG | 192 |
| | GGgttttagagctagaaatagcaagtta | | AAGCgttttagagctagaaatagca | |
| | aaataaggctagtccgttatcaacttgaa | | agttaaaataaggctagtccgttatcaac | |
| | aaagtggcaccGAGTCGGTGC | | ttgaaaaagtggcaccGAGTCGG | |
| | CAGCCCTCGCCTCCTATG | | TGC | |
| | ATCCTGACGACGGAGAC | | | |
| | CGCCGTCGTCGACAAGC | | | |
| | C | | | |
| 17 | gCTTTAGAAGTTAAGGAG | 193 | gAGGAAGGTCCGCAGAG | 194 |
| | GCGgttttagagctagaaatagcaagtt | | AAGCgttttagagctagaaatagca | |
| | aaaataaggctagtccgttatcaacttga | | agttaaaataaggctagtccgttatcaac | |
| | aaaagtggcaccGAGTCGGTGC | | ttgaaaaagtggcaccGAGTCGG | |
| | GACCCCAGCCCTCGCAT | | TGC | |
| | GATCCTGACGACGGAGA | | | |
| | CCGCCGTCGTCGACAAG | | | |
| | CC | | | |
| 18 | gTTTAGAAGTTAAGGAGG | 195 | gAGGAAGGTCCGCAGAG | 196 |
| | CGAgttttagagctagaaatagcaagtt | | AAGCgttttagagctagaaatagca | |
| | aaaataaggctagtccgttatcaacttga | | agttaaaataaggctagtccgttatcaac | |
| | aaaagtggcaccGAGTCGGTGC | | ttgaaaaagtggcaccGAGTCGG | |
| | AGACCCCAGCCCTCGAT | | TGC | |
| | GATCCTGACGACGGAGA | | | |
| | CCGCCGTCGTCGACAAG | | | |
| | CC | | | |
| 19 | GAAGTTAAGGAGGCGAG | 197 | gAGGAAGGTCCGCAGAG | 198 |
| | GGCgttttagagctagaaatagcaagtt | | AAGCgttttagagctagaaatagca | |
| | aaaataaggctagtccgttatcaacttga | | agttaaaataaggctagtccgttatcaac | |
| | aaaagtggcaccGAGTCGGTGC | | ttgaaaaagtggcaccGAGTCGG | |
| | TGACAGACCCCAGCCAT | | TGC | |
| | GATCCTGACGACGGAGA | | | |
| | CCGCCGTCGTCGACAAG | | | |
| | CC | | | |
| 20 | gAAGTTAAGGAGGCGAG | 199 | gAGGAAGGTCCGCAGAG | 200 |
| | GGCTgttttagagctagaaatagcaa | | AAGCgttttagagctagaaatagca | |
| | gttaaaataaggctagtccgttatcaactt | | agttaaaataaggctagtccgttatcaac | |
| | gaaaaagtggcaccGAGTCGGT | | ttgaaaaagtggcaccGAGTCGG | |
| | GCCTGACAGACCCCAGC | | TGC | |
| | ATGATCCTGACGACGGA | | | |
| | GACCGCCGTCGTCGACA | | | |
| | AGCC | | | |
| 21 | gAGTTAAGGAGGCGAGG | 201 | gAGGAAGGTCCGCAGAG | 202 |
| | GCTGgttttagagctagaaatagcaa | | AAGCgttttagagctagaaatagca | |
| | gttaaaataaggctagtccgttatcaactt | | agttaaaataaggctagtccgttatcaac | |
| | gaaaaagtggcaccGAGTCGGT | | ttgaaaaagtggcaccGAGTCGG | |
| | GCACTGACAGACCCCAG | | TGC | |
| | ATGATCCTGACGACGGA | | | |
| | GACCGCCGTCGTCGACA | | | |
| | AGCC | | | |
| 22 | gCTTGTCGGCTTTAGAAG | 203 | gCGAGACCTCCAGCCTG | 204 |
| | TTAgttttagagctagaaatagcaagtt | | AGGAgttttagagctagaaatagca | |
| | aaaataaggctagtccgttatcaacttga | | agttaaaataaggctagtccgttatcaac | |
| | aaaagtggcaccGAGTCGGTGC | | ttgaaaaagtggcaccGAGTCGG | |
| | CCCTCGCCTCCTTAAATG | | TGC | |
| | ATCCTGACGACGGAGAC | | | |
| | CGCCGTCGTCGACAAGC | | | |
| | C | | | |
| 23 | GTCGGCTTTAGAAGTTAA | 205 | gCGAGACCTCCAGCCTG | 206 |
| | GGgttttagagctagaaatagcaagtta | | AGGAgttttagagctagaaatagca | |
| | aaataaggctagtccgttatcaacttgaa | | agttaaaataaggctagtccgttatcaac | |
| | aaagtggcaccGAGTCGGTGC | | ttgaaaaagtggcaccGAGTCGG | |
| | CAGCCCTCGCCTCCTATG | | TGC | |
| | ATCCTGACGACGGAGAC | | | |
| | CGCCGTCGTCGACAAGC | | | |
| | C | | | |
| 24 | gCTTTAGAAGTTAAGGAG | 207 | gCGAGACCTCCAGCCTG | 208 |
| | GCGgttttagagctagaaatagcaagtt | | AGGAgttttagagctagaaatagca | |
| | aaaataaggctagtccgttatcaacttga | | agttaaaataaggctagtccgttatcaac | |
| | aaaagtggcaccGAGTCGGTGC | | ttgaaaaagtggcaccGAGTCGG | |
| | GACCCCAGCCCTCGCAT | | TGC | |
| | GATCCTGACGACGGAGA | | | |
| | CCGCCGTCGTCGACAAG | | | |
| | CC | | | |
| 25 | gTTTAGAAGTTAAGGAGG | 209 | gCGAGACCTCCAGCCTG | 210 |
| | CGAgttttagagctagaaatagcaagtt | | AGGAgttttagagctagaaatagca | |
| | aaaataaggctagtccgttatcaacttga | | agttaaaataaggctagtccgttatcaac | |
| | aaaagtggcaccGAGTCGGTGC | | ttgaaaaagtggcaccGAGTCGG | |
| | AGACCCCAGCCCTCGAT | | TGC | |
| | GATCCTGACGACGGAGA | | | |
| | CCGCCGTCGTCGACAAG | | | |
| | CC | | | |
| 26 | GAAGTTAAGGAGGCGAG | 211 | gCGAGACCTCCAGCCTG | 212 |
| | GGCgttttagagctagaaatagcaagtt | | AGGAgttttagagctagaaatagca | |
| | aaaataaggctagtccgttatcaacttga | | agttaaaataaggctagtccgttatcaac | |
| | aaaagtggcaccGAGTCGGTGC | | ttgaaaaagtggcaccGAGTCGG | |
| | TGACAGACCCCAGCCAT | | TGC | |
| | GATCCTGACGACGGAGA | | | |
| | CCGCCGTCGTCGACAAG | | | |
| | CC | | | |
| 27 | gAAGTTAAGGAGGCGAG | 213 | gCGAGACCTCCAGCCTG | 214 |
| | GGCTgttttagagctagaaatagcaa | | AGGAgttttagagctagaaatagca | |
| | gttaaaataaggctagtccgttatcaactt | | agttaaaataaggctagtccgttatcaac | |
| | gaaaaagtggcaccGAGTCGGT | | ttgaaaaagtggcaccGAGTCGG | |
| | GCCTGACAGACCCCAGC | | TGC | |
| | ATGATCCTGACGACGGA | | | |
| | GACCGCCGTCGTCGACA | | | |
| | AGCC | | | |
| 28 | gAGTTAAGGAGGCGAGG | 215 | gCGAGACCTCCAGCCTG | 216 |
| | GCTGgttttagagctagaaatagcaa | | AGGAgttttagagctagaaatagca | |
| | gttaaaataaggctagtccgttatcaactt | | agttaaaataaggctagtccgttatcaac | |
| | gaaaaagtggcaccGAGTCGGT | | ttgaaaaagtggcaccGAGTCGG | |
| | GCACTGACAGACCCCAG | | TGC | |
| | ATGATCCTGACGACGGA | | | |
| | GACCGCCGTCGTCGACA | | | |
| | AGCC | | | |
| 29 | gCTTGTCGGCTTTAGAAG | 217 | gACACCGAGACCTCCAG | 218 |
| | TTAgttttagagctagaaatagcaagtt | | CCTGgttttagagctagaaatagcaa | |
| | aaaataaggctagtccgttatcaacttga | | gttaaaataaggctagtccgttatcaact | |
| | aaaagtggcaccGAGTCGGTGC | | tgaaaaagtggcaccGAGTCGG | |
| | CCCTCGCCTCCTTAAATG | | TGC | |
| | ATCCTGACGACGGAGAC | | | |
| | CGCCGTCGTCGACAAGC | | | |
| | C | | | |
| 30 | GTCGGCTTTAGAAGTTAA | 219 | gACACCGAGACCTCCAG | 220 |
| | GGgttttagagctagaaatagcaagtta | | CCTGgttttagagctagaaatagcaa | |
| | aaataaggctagtccgttatcaacttgaa | | gttaaaataaggctagtccgttatcaact | |
| | aaagtggcaccGAGTCGGTGC | | tgaaaaagtggcaccGAGTCGG | |
| | CAGCCCTCGCCTCCTATG | | TGC | |
| | ATCCTGACGACGGAGAC | | | |
| | CGCCGTCGTCGACAAGC | | | |
| | C | | | |
| 31 | gCTTTAGAAGTTAAGGAG | 221 | gACACCGAGACCTCCAG | 222 |
| | GCGgttttagagctagaaatagcaagtt | | CCTGgttttagagctagaaatagcaa | |
| | aaaataaggctagtccgttatcaacttga | | gttaaaataaggctagtccgttatcaact | |
| | aaaagtggcaccGAGTCGGTGC | | tgaaaaagtggcaccGAGTCGG | |
| | GACCCCAGCCCTCGCAT | | TGC | |
| | GATCCTGACGACGGAGA | | | |
| | CCGCCGTCGTCGACAAG | | | |
| | CC | | | |
| 32 | gTTTAGAAGTTAAGGAGG | 223 | gACACCGAGACCTCCAG | 224 |
| | CGAgttttagagctagaaatagcaagtt | | CCTGgttttagagctagaaatagcaa | |
| | aaaataaggctagtccgttatcaacttga | | gttaaaataaggctagtccgttatcaact | |
| | aaaagtggcaccGAGTCGGTGC | | tgaaaaagtggcaccGAGTCGG | |
| | AGACCCCAGCCCTCGAT | | TGC | |
| | GATCCTGACGACGGAGA | | | |
| | CCGCCGTCGTCGACAAG | | | |
| | CC | | | |
| 33 | GAAGTTAAGGAGGCGAG | 225 | gACACCGAGACCTCCAG | 226 |
| | GGCgttttagagctagaaatagcaagtt | | CCTGgttttagagctagaaatagcaa | |
| | aaaataaggctagtccgttatcaacttga | | gttaaaataaggctagtccgttatcaact | |
| | aaaagtggcaccGAGTCGGTGC | | tgaaaaagtggcaccGAGTCGG | |
| | TGACAGACCCCAGCCAT | | TGC | |
| | GATCCTGACGACGGAGA | | | |
| | CCGCCGTCGTCGACAAG | | | |
| | CC | | | |
| 34 | gAAGTTAAGGAGGCGAG | 227 | gACACCGAGACCTCCAG | 228 |
| | GGCTgttttagagctagaaatagcaa | | CCTGgttttagagctagaaatagcaa | |
| | gttaaaataaggctagtccgttatcaactt | | gttaaaataaggctagtccgttatcaact | |
| | gaaaaagtggcaccGAGTCGGT | | tgaaaaagtggcaccGAGTCGG | |
| | GCCTGACAGACCCCAGC | | TGC | |
| | ATGATCCTGACGACGGA | | | |
| | GACCGCCGTCGTCGACA | | | |
| | AGCC | | | |
| 35 | gAGTTAAGGAGGCGAGG | 229 | gACACCGAGACCTCCAG | 230 |
| | GCTGgttttagagctagaaatagcaa | | CCTGgttttagagctagaaatagcaa | |
| | gttaaaataaggctagtccgttatcaactt | | gttaaaataaggctagtccgttatcaact | |
| | gaaaaagtggcaccGAGTCGGT | | tgaaaaagtggcaccGAGTCGG | |
| | GCACTGACAGACCCCAG | | TGC | |
| | ATGATCCTGACGACGGA | | | |
| | GACCGCCGTCGTCGACA | | | |
| | AGCC | | | |
| 36 | gCTTGTCGGCTTTAGAAG | 231 | gAGCTAGTCAGACATGG | 232 |
| | TTAgttttagagctagaaatagcaagtt | | TGGAgttttagagctagaaatagcaa | |
| | aaaataaggctagtccgttatcaacttga | | gttaaaataaggctagtccgttatcaact | |
| | aaaagtggcaccGAGTCGGTGC | | tgaaaaagtggcaccGAGTCGG | |
| | CCCTCGCCTCCTTAAATG | | TGC | |
| | ATCCTGACGACGGAGAC | | | |
| | CGCCGTCGTCGACAAGC | | | |
| | C | | | |
| 37 | GTCGGCTTTAGAAGTTAA | 233 | gAGCTAGTCAGACATGG | 234 |
| | GGgttttagagctagaaatagcaagtta | | TGGAgttttagagctagaaatagcaa | |
| | aaataaggctagtccgttatcaacttgaa | | gttaaaataaggctagtccgttatcaact | |
| | aaagtggcaccGAGTCGGTGC | | tgaaaaagtggcaccGAGTCGG | |
| | CAGCCCTCGCCTCCTATG | | TGC | |
| | ATCCTGACGACGGAGAC | | | |
| | CGCCGTCGTCGACAAGC | | | |
| | C | | | |
| 38 | gCTTTAGAAGTTAAGGAG | 235 | gAGCTAGTCAGACATGG | 236 |
| | GCGgttttagagctagaaatagcaagtt | | TGGAgttttagagctagaaatagcaa | |
| | aaaataaggctagtccgttatcaacttga | | gttaaaataaggctagtccgttatcaact | |
| | aaaagtggcaccGAGTCGGTGC | | tgaaaaagtggcaccGAGTCGG | |
| | GACCCCAGCCCTCGCAT | | TGC | |
| | GATCCTGACGACGGAGA | | | |
| | CCGCCGTCGTCGACAAG | | | |
| | CC | | | |
| 39 | gTTTAGAAGTTAAGGAGG | 237 | gAGCTAGTCAGACATGG | 238 |
| | CGAgttttagagctagaaatagcaagtt | | TGGAgttttagagctagaaatagcaa | |
| | aaaataaggctagtccgttatcaacttga | | gttaaaataaggctagtccgttatcaact | |
| | aaaagtggcaccGAGTCGGTGC | | tgaaaaagtggcaccGAGTCGG | |
| | AGACCCCAGCCCTCGAT | | TGC | |
| | GATCCTGACGACGGAGA | | | |
| | CCGCCGTCGTCGACAAG | | | |
| | CC | | | |
| 40 | GAAGTTAAGGAGGCGAG | 239 | gAGCTAGTCAGACATGG | 240 |
| | GGCgttttagagctagaaatagcaagtt | | TGGAgttttagagctagaaatagcaa | |
| | aaaataaggctagtccgttatcaacttga | | gttaaaataaggctagtccgttatcaact | |
| | aaaagtggcaccGAGTCGGTGC | | tgaaaaagtggcaccGAGTCGG | |
| | TGACAGACCCCAGCCAT | | TGC | |
| | GATCCTGACGACGGAGA | | | |
| | CCGCCGTCGTCGACAAG | | | |
| | CC | | | |
| 41 | gAAGTTAAGGAGGCGAG | 241 | gAGCTAGTCAGACATGG | 242 |
| | GGCTgttttagagctagaaatagcaa | | TGGAgttttagagctagaaatagcaa | |
| | gttaaaataaggctagtccgttatcaactt | | gttaaaataaggctagtccgttatcaact | |
| | gaaaaagtggcaccGAGTCGGT | | tgaaaaagtggcaccGAGTCGG | |
| | GCCTGACAGACCCCAGC | | TGC | |
| | ATGATCCTGACGACGGA | | | |
| | GACCGCCGTCGTCGACA | | | |
| | AGCC | | | |
| 42 | gAGTTAAGGAGGCGAGG | 243 | gAGCTAGTCAGACATGG | 244 |
| | GCTGgttttagagctagaaatagcaa | | TGGAgttttagagctagaaatagcaa | |
| | gttaaaataaggctagtccgttatcaactt | | gttaaaataaggctagtccgttatcaact | |
| | gaaaaagtggcaccGAGTCGGT | | tgaaaaagtggcaccGAGTCGG | |
| | GCACTGACAGACCCCAG | | TGC | |
| | ATGATCCTGACGACGGA | | | |
| | GACCGCCGTCGTCGACA | | | |
| | AGCC | | | |
| 43 | gCTTGTCGGCTTTAGAAG | 245 | gAGCTAGCTAGTCAGAC | 246 |
| | TTAgttttagagctagaaatagcaagtt | | ATGGgttttagagctagaaatagcaa | |
| | aaaataaggctagtccgttatcaacttga | | gttaaaataaggctagtccgttatcaact | |
| | aaaagtggcaccGAGTCGGTGC | | tgaaaaagtggcaccGAGTCGG | |
| | CCCTCGCCTCCTTAAATG | | TGC | |
| | ATCCTGACGACGGAGAC | | | |
| | CGCCGTCGTCGACAAGC | | | |
| | C | | | |
| 44 | GTCGGCTTTAGAAGTTAA | 247 | gAGCTAGCTAGTCAGAC | 248 |
| | GGgttttagagctagaaatagcaagtta | | ATGGgttttagagctagaaatagcaa | |
| | aaataaggctagtccgttatcaacttgaa | | gttaaaataaggctagtccgttatcaact | |
| | aaagtggcaccGAGTCGGTGC | | tgaaaaagtggcaccGAGTCGG | |
| | CAGCCCTCGCCTCCTATG | | TGC | |
| | ATCCTGACGACGGAGAC | | | |
| | CGCCGTCGTCGACAAGC | | | |
| | C | | | |
| 45 | gCTTTAGAAGTTAAGGAG | 249 | gAGCTAGCTAGTCAGAC | 250 |
| | GCGgttttagagctagaaatagcaagtt | | ATGGgttttagagctagaaatagcaa | |
| | aaaataaggctagtccgttatcaacttga | | gttaaaataaggctagtccgttatcaact | |
| | aaaagtggcaccGAGTCGGTGC | | tgaaaaagtggcaccGAGTCGG | |
| | GACCCCAGCCCTCGCAT | | TGC | |
| | GATCCTGACGACGGAGA | | | |
| | CCGCCGTCGTCGACAAG | | | |
| | CC | | | |
| 46 | gTTTAGAAGTTAAGGAGG | 251 | gAGCTAGCTAGTCAGAC | 252 |
| | CGAgttttagagctagaaatagcaagtt | | ATGGgttttagagctagaaatagcaa | |
| | aaaataaggctagtccgttatcaacttga | | gttaaaataaggctagtccgttatcaact | |
| | aaaagtggcaccGAGTCGGTGC | | tgaaaaagtggcaccGAGTCGG | |
| | AGACCCCAGCCCTCGAT | | TGC | |
| | GATCCTGACGACGGAGA | | | |
| | CCGCCGTCGTCGACAAG | | | |
| | CC | | | |
| 47 | GAAGTTAAGGAGGCGAG | 253 | gAGCTAGCTAGTCAGAC | 254 |
| | GGCgttttagagctagaaatagcaagtt | | ATGGgttttagagctagaaatagcaa | |
| | aaaataaggctagtccgttatcaacttga | | gttaaaataaggctagtccgttatcaact | |
| | aaaagtggcaccGAGTCGGTGC | | tgaaaaagtggcaccGAGTCGG | |
| | TGACAGACCCCAGCCAT | | TGC | |
| | GATCCTGACGACGGAGA | | | |
| | CCGCCGTCGTCGACAAG | | | |
| | CC | | | |
| 48 | gAAGTTAAGGAGGCGAG | 255 | gAGCTAGCTAGTCAGAC | 256 |
| | GGCTgttttagagctagaaatagcaa | | ATGGgttttagagctagaaatagcaa | |
| | gttaaaataaggctagtccgttatcaactt | | gttaaaataaggctagtccgttatcaact | |
| | gaaaaagtggcaccGAGTCGGT | | tgaaaaagtggcaccGAGTCGG | |
| | GCCTGACAGACCCCAGC | | TGC | |
| | ATGATCCTGACGACGGA | | | |
| | GACCGCCGTCGTCGACA | | | |
| | AGCC | | | |
| 49 | gAGTTAAGGAGGCGAGG | 257 | gAGCTAGCTAGTCAGAC | 258 |
| | GCTGgttttagagctagaaatagcaa | | ATGGgttttagagctagaaatagcaa | |
| | gttaaaataaggctagtccgttatcaactt | | gttaaaataaggctagtccgttatcaact | |
| | gaaaaagtggcaccGAGTCGGT | | tgaaaaagtggcaccGAGTCGG | |
| | GCACTGACAGACCCCAG | | TGC | |
| | ATGATCCTGACGACGGA | | | |
| | GACCGCCGTCGTCGACA | | | |
| | AGCC | | | |
| 50 | gCTTGTCGGCTTTAGAAG | 259 | gCAGAGAAGCTGGGCAG | 260 |
| | TTAgttttagagctagaaatagcaagtt | | ACAAgttttagagctagaaatagca | |
| | aaaataaggctagtccgttatcaacttga | | agttaaaataaggctagtccgttatcaac | |
| | aaaagtggcaccGAGTCGGTGC | | ttgaaaaagtggcaccGAGTCGG | |
| | CCCTCGCCTCCTTAAGGC | | TGC | |
| | TTGTCGACGACGGCGGT | | | |
| | CTCCGTCGTCAGGATCAT | | | |
| 51 | GTCGGCTTTAGAAGTTAA | 261 | gCAGAGAAGCTGGGCAG | 262 |
| | GGgttttagagctagaaatagcaagtta | | ACAAgttttagagctagaaatagca | |
| | aaataaggctagtccgttatcaacttgaa | | agttaaaataaggctagtccgttatcaac | |
| | aaagtggcaccGAGTCGGTGC | | ttgaaaaagtggcaccGAGTCGG | |
| | CAGCCCTCGCCTCCTGGC | | TGC | |
| | TTGTCGACGACGGCGGT | | | |
| | CTCCGTCGTCAGGATCAT | | | |
| 52 | gCTTTAGAAGTTAAGGAG | 263 | gCAGAGAAGCTGGGCAG | 264 |
| | GCGgttttagagctagaaatagcaagtt | | ACAAgttttagagctagaaatagca | |
| | aaaataaggctagtccgttatcaacttga | | agttaaaataaggctagtccgttatcaac | |
| | aaaagtggcaccGAGTCGGTGC | | | |
| | GACCCCAGCCCTCGCGG | | ttgaaaaagtggcaccGAGTCGG | |
| | CTTGTCGACGACGGCGG | | TGC | |
| | TCTCCGTCGTCAGGATCA | | | |
| | T | | | |
| 53 | gTTTAGAAGTTAAGGAGG | 265 | gCAGAGAAGCTGGGCAG | 266 |
| | CGAgttttagagctagaaatagcaagtt | | ACAAgttttagagctagaaatagca | |
| | aaaataaggctagtccgttatcaacttga | | agttaaaataaggctagtccgttatcaac | |
| | aaaagtggcaccGAGTCGGTGC | | ttgaaaaagtggcaccGAGTCGG | |
| | AGACCCCAGCCCTCGGG | | TGC | |
| | CTTGTCGACGACGGCGG | | | |
| | TCTCCGTCGTCAGGATCA | | | |
| | T | | | |
| 54 | GAAGTTAAGGAGGCGAG | 267 | gCAGAGAAGCTGGGCAG | 268 |
| | GGCgttttagagctagaaatagcaagtt | | ACAAgttttagagctagaaatagca | |
| | aaaataaggctagtccgttatcaacttga | | agttaaaataaggctagtccgttatcaac | |
| | aaaagtggcaccGAGTCGGTGC | | ttgaaaaagtggcaccGAGTCGG | |
| | TGACAGACCCCAGCCGG | | TGC | |
| | CTTGTCGACGACGGCGG | | | |
| | TCTCCGTCGTCAGGATCA | | | |
| | T | | | |
| 55 | gAAGTTAAGGAGGCGAG | 269 | gCAGAGAAGCTGGGCAG | 270 |
| | GGCTgttttagagctagaaatagcaa | | ACAAgttttagagctagaaatagca | |
| | gttaaaataaggctagtccgttatcaactt | | agttaaaataaggctagtccgttatcaac | |
| | gaaaaagtggcaccGAGTCGGT | | ttgaaaaagtggcaccGAGTCGG | |
| | GCCTGACAGACCCCAGC | | TGC | |
| | GGCTTGTCGACGACGGC | | | |
| | GGTCTCCGTCGTCAGGAT | | | |
| | CAT | | | |
| 56 | gAGTTAAGGAGGCGAGG | 271 | gCAGAGAAGCTGGGCAG | 272 |
| | GCTGgttttagagctagaaatagcaa | | ACAAgttttagagctagaaatagca | |
| | gttaaaataaggctagtccgttatcaactt | | agttaaaataaggctagtccgttatcaac | |
| | gaaaaagtggcaccGAGTCGGT | | ttgaaaaagtggcaccGAGTCGG | |
| | GCACTGACAGACCCCAG | | TGC | |
| | GGCTTGTCGACGACGGC | | | |
| | GGTCTCCGTCGTCAGGAT | | | |
| | CAT | | | |
| 57 | gCTTGTCGGCTTTAGAAG | 273 | GGAAGGTCCGCAGAGA | 274 |
| | TTAgttttagagctagaaatagcaagtt | | AGCTgttttagagctagaaatagcaa | |
| | aaaataaggctagtccgttatcaacttga | | gttaaaataaggctagtccgttatcaact | |
| | aaaagtggcaccGAGTCGGTGC | | tgaaaaagtggcaccGAGTCGG | |
| | CCCTCGCCTCCTTAAGGC | | TGC | |
| | TTGTCGACGACGGCGGT | | | |
| | CTCCGTCGTCAGGATCAT | | | |
| 58 | GTCGGCTTTAGAAGTTAA | 275 | GGAAGGTCCGCAGAGA | 276 |
| | GGgttttagagctagaaatagcaagtta | | AGCTgttttagagctagaaatagcaa | |
| | aaataaggctagtccgttatcaacttgaa | | gttaaaataaggctagtccgttatcaact | |
| | aaagtggcaccGAGTCGGTGC | | tgaaaaagtggcaccGAGTCGG | |
| | CAGCCCTCGCCTCCTGGC | | TGC | |
| | TTGTCGACGACGGCGGT | | | |
| | CTCCGTCGTCAGGATCAT | | | |
| 59 | gCTTTAGAAGTTAAGGAG | 277 | GGAAGGTCCGCAGAGA | 278 |
| | GCGgttttagagctagaaatagcaagtt | | AGCTgttttagagctagaaatagcaa | |
| | aaaataaggctagtccgttatcaacttga | | gttaaaataaggctagtccgttatcaact | |
| | aaaagtggcaccGAGTCGGTGC | | tgaaaaagtggcaccGAGTCGG | |
| | GACCCCAGCCCTCGCGG | | TGC | |
| | CTTGTCGACGACGGCGG | | | |
| | TCTCCGTCGTCAGGATCA | | | |
| | T | | | |
| 60 | gTTTAGAAGTTAAGGAGG | 279 | GGAAGGTCCGCAGAGA | 280 |
| | CGAgttttagagctagaaatagcaagtt | | AGCTgttttagagctagaaatagcaa | |
| | aaaataaggctagtccgttatcaacttga | | gttaaaataaggctagtccgttatcaact | |
| | aaaagtggcaccGAGTCGGTGC | | tgaaaaagtggcaccGAGTCGG | |
| | AGACCCCAGCCCTCGGG | | TGC | |
| | CTTGTCGACGACGGCGG | | | |
| | TCTCCGTCGTCAGGATCA | | | |
| | T | | | |
| 61 | GAAGTTAAGGAGGCGAG | 281 | GGAAGGTCCGCAGAGA | 282 |
| | GGCgttttagagctagaaatagcaagtt | | AGCTgttttagagctagaaatagcaa | |
| | aaaataaggctagtccgttatcaacttga | | gttaaaataaggctagtccgttatcaact | |
| | aaaagtggcaccGAGTCGGTGC | | tgaaaaagtggcaccGAGTCGG | |
| | TGACAGACCCCAGCCGG | | TGC | |
| | CTTGTCGACGACGGCGG | | | |
| | TCTCCGTCGTCAGGATCA | | | |
| | T | | | |
| 62 | gAAGTTAAGGAGGCGAG | 283 | GGAAGGTCCGCAGAGA | 284 |
| | GGCTgttttagagctagaaatagcaa | | AGCTgttttagagctagaaatagcaa | |
| | gttaaaataaggctagtccgttatcaactt | | gttaaaataaggctagtccgttatcaact | |
| | gaaaaagtggcaccGAGTCGGT | | tgaaaaagtggcaccGAGTCGG | |
| | GCCTGACAGACCCCAGC | | TGC | |
| | GGCTTGTCGACGACGGC | | | |
| | GGTCTCCGTCGTCAGGAT | | | |
| | CAT | | | |
| 63 | gAGTTAAGGAGGCGAGG | 285 | GGAAGGTCCGCAGAGA | 286 |
| | GCTGgttttagagctagaaatagcaa | | AGCTgttttagagctagaaatagcaa | |
| | gttaaaataaggctagtccgttatcaactt | | gttaaaataaggctagtccgttatcaact | |
| | gaaaaagtggcaccGAGTCGGT | | tgaaaaagtggcaccGAGTCGG | |
| | GCACTGACAGACCCCAG | | TGC | |
| | GGCTTGTCGACGACGGC | | | |
| | GGTCTCCGTCGTCAGGAT | | | |
| | CAT | | | |
| 64 | gCTTGTCGGCTTTAGAAG | 287 | gAGGAAGGTCCGCAGAG | 288 |
| | TTAgttttagagctagaaatagcaagtt | | AAGCgttttagagctagaaatagca | |
| | aaaataaggctagtccgttatcaacttga | | agttaaaataaggctagtccgttatcaac | |
| | aaaagtggcaccGAGTCGGTGC | | ttgaaaaagtggcaccGAGTCGG | |
| | CCCTCGCCTCCTTAAGGC | | TGC | |
| | TTGTCGACGACGGCGGT | | | |
| | CTCCGTCGTCAGGATCAT | | | |
| 65 | GTCGGCTTTAGAAGTTAA | 289 | gAGGAAGGTCCGCAGAG | 290 |
| | GGgttttagagctagaaatagcaagtta | | AAGCgttttagagctagaaatagca | |
| | aaataaggctagtccgttatcaacttgaa | | agttaaaataaggctagtccgttatcaac | |
| | aaagtggcaccGAGTCGGTGC | | ttgaaaaagtggcaccGAGTCGG | |
| | CAGCCCTCGCCTCCTGGC | | TGC | |
| | TTGTCGACGACGGCGGT | | | |
| | CTCCGTCGTCAGGATCAT | | | |
| 66 | gCTTTAGAAGTTAAGGAG | 291 | gAGGAAGGTCCGCAGAG | 292 |
| | GCGgttttagagctagaaatagcaagtt | | AAGCgttttagagctagaaatagca | |
| | aaaataaggctagtccgttatcaacttga | | agttaaaataaggctagtccgttatcaac | |
| | aaaagtggcaccGAGTCGGTGC | | ttgaaaaagtggcaccGAGTCGG | |
| | GACCCCAGCCCTCGCGG | | TGC | |
| | CTTGTCGACGACGGCGG | | | |
| | TCTCCGTCGTCAGGATCA | | | |
| | T | | | |
| 67 | gTTTAGAAGTTAAGGAGG | 293 | gAGGAAGGTCCGCAGAG | 294 |
| | CGAgttttagagctagaaatagcaagtt | | AAGCgttttagagctagaaatagca | |
| | aaaataaggctagtccgttatcaacttga | | agttaaaataaggctagtccgttatcaac | |
| | aaaagtggcaccGAGTCGGTGC | | ttgaaaaagtggcaccGAGTCGG | |
| | AGACCCCAGCCCTCGGG | | TGC | |
| | CTTGTCGACGACGGCGG | | | |
| | TCTCCGTCGTCAGGATCA | | | |
| | T | | | |
| 68 | GAAGTTAAGGAGGCGAG | 295 | gAGGAAGGTCCGCAGAG | 296 |
| | GGCgttttagagctagaaatagcaagtt | | AAGCgttttagagctagaaatagca | |
| | aaaataaggctagtccgttatcaacttga | | agttaaaataaggctagtccgttatcaac | |
| | aaaagtggcaccGAGTCGGTGC | | ttgaaaaagtggcaccGAGTCGG | |
| | TGACAGACCCCAGCCGG | | TGC | |
| | CTTGTCGACGACGGCGG | | | |
| | TCTCCGTCGTCAGGATCA | | | |
| | T | | | |
| 69 | gAAGTTAAGGAGGCGAG | 297 | gAGGAAGGTCCGCAGAG | 298 |
| | GGCTgttttagagctagaaatagcaa | | AAGCgttttagagctagaaatagca | |
| | gttaaaataaggctagtccgttatcaactt | | agttaaaataaggctagtccgttatcaac | |
| | gaaaaagtggcaccGAGTCGGT | | ttgaaaaagtggcaccGAGTCGG | |
| | GCCTGACAGACCCCAGC | | TGC | |
| | GGCTTGTCGACGACGGC | | | |
| | GGTCTCCGTCGTCAGGAT | | | |
| | CAT | | | |
| 70 | gAGTTAAGGAGGCGAGG | 299 | gAGGAAGGTCCGCAGAG | 300 |
| | GCTGgttttagagctagaaatagcaa | | AAGCgttttagagctagaaatagca | |
| | gttaaaataaggctagtccgttatcaactt | | agttaaaataaggctagtccgttatcaac | |
| | gaaaaagtggcaccGAGTCGGT | | ttgaaaaagtggcaccGAGTCGG | |
| | GCACTGACAGACCCCAG | | TGC | |
| | GGCTTGTCGACGACGGC | | | |
| | GGTCTCCGTCGTCAGGAT | | | |
| | CAT | | | |
| 71 | gCTTGTCGGCTTTAGAAG | 301 | gCGAGACCTCCAGCCTG | 302 |
| | TTAgttttagagctagaaatagcaagtt | | AGGAgttttagagctagaaatagca | |
| | aaaataaggctagtccgttatcaacttga | | agttaaaataaggctagtccgttatcaac | |
| | aaaagtggcaccGAGTCGGTGC | | ttgaaaaagtggcaccGAGTCGG | |
| | CCCTCGCCTCCTTAAGGC | | TGC | |
| | TTGTCGACGACGGCGGT | | | |
| | CTCCGTCGTCAGGATCAT | | | |
| 72 | GTCGGCTTTAGAAGTTAA | 303 | gCGAGACCTCCAGCCTG | 304 |
| | GGgttttagagctagaaatagcaagtta | | AGGAgttttagagctagaaatagca | |
| | aaataaggctagtccgttatcaacttgaa | | agttaaaataaggctagtccgttatcaac | |
| | aaagtggcaccGAGTCGGTGC | | ttgaaaaagtggcaccGAGTCGG | |
| | CAGCCCTCGCCTCCTGGC | | TGC | |
| | TTGTCGACGACGGCGGT | | | |
| | CTCCGTCGTCAGGATCAT | | | |
| 73 | gCTTTAGAAGTTAAGGAG | 305 | gCGAGACCTCCAGCCTG | 306 |
| | GCGgttttagagctagaaatagcaagtt | | AGGAgttttagagctagaaatagca | |
| | aaaataaggctagtccgttatcaacttga | | agttaaaataaggctagtccgttatcaac | |
| | aaaagtggcaccGAGTCGGTGC | | ttgaaaaagtggcaccGAGTCGG | |
| | GACCCCAGCCCTCGCGG | | TGC | |
| | CTTGTCGACGACGGCGG | | | |
| | TCTCCGTCGTCAGGATCA | | | |
| | T | | | |
| 74 | gTTTAGAAGTTAAGGAGG | 307 | gCGAGACCTCCAGCCTG | 308 |
| | CGAgttttagagctagaaatagcaagtt | | AGGAgttttagagctagaaatagca | |
| | aaaataaggctagtccgttatcaacttga | | agttaaaataaggctagtccgttatcaac | |
| | aaaagtggcaccGAGTCGGTGC | | ttgaaaaagtggcaccGAGTCGG | |
| | AGACCCCAGCCCTCGGG | | TGC | |
| | CTTGTCGACGACGGCGG | | | |
| | TCTCCGTCGTCAGGATCA | | | |
| | T | | | |
| 75 | GAAGTTAAGGAGGCGAG | 309 | gCGAGACCTCCAGCCTG | 310 |
| | GGCgttttagagctagaaatagcaagtt | | AGGAgttttagagctagaaatagca | |
| | aaaataaggctagtccgttatcaacttga | | agttaaaataaggctagtccgttatcaac | |
| | aaaagtggcaccGAGTCGGTGC | | ttgaaaaagtggcaccGAGTCGG | |
| | TGACAGACCCCAGCCGG | | TGC | |
| | CTTGTCGACGACGGCGG | | | |
| | TCTCCGTCGTCAGGATCA | | | |
| | T | | | |
| 76 | gAAGTTAAGGAGGCGAG | 311 | gCGAGACCTCCAGCCTG | 312 |
| | GGCTgttttagagctagaaatagcaa | | AGGAgttttagagctagaaatagca | |
| | gttaaaataaggctagtccgttatcaactt | | agttaaaataaggctagtccgttatcaac | |
| | gaaaaagtggcaccGAGTCGGT | | ttgaaaaagtggcaccGAGTCGG | |
| | GCCTGACAGACCCCAGC | | TGC | |
| | GGCTTGTCGACGACGGC | | | |
| | GGTCTCCGTCGTCAGGAT | | | |
| | CAT | | | |
| 77 | gAGTTAAGGAGGCGAGG | 313 | gCGAGACCTCCAGCCTG | 314 |
| | GCTGgttttagagctagaaatagcaa | | AGGAgttttagagctagaaatagca | |
| | gttaaaataaggctagtccgttatcaactt | | agttaaaataaggctagtccgttatcaac | |
| | gaaaaagtggcaccGAGTCGGT | | ttgaaaaagtggcaccGAGTCGG | |
| | GCACTGACAGACCCCAG | | TGC | |
| | GGCTTGTCGACGACGGC | | | |
| | GGTCTCCGTCGTCAGGAT | | | |
| | CAT | | | |
| 78 | gCTTGTCGGCTTTAGAAG | 315 | gACACCGAGACCTCCAG | 316 |
| | TTAgttttagagctagaaatagcaagtt | | CCTGgttttagagctagaaatagcaa | |
| | aaaataaggctagtccgttatcaacttga | | gttaaaataaggctagtccgttatcaact | |
| | aaaagtggcaccGAGTCGGTGC | | tgaaaaagtggcaccGAGTCGG | |
| | CCCTCGCCTCCTTAAGGC | | TGC | |
| | TTGTCGACGACGGCGGT | | | |
| | CTCCGTCGTCAGGATCAT | | | |
| 79 | GTCGGCTTTAGAAGTTAA | 317 | gACACCGAGACCTCCAG | 318 |
| | GGgttttagagctagaaatagcaagtta | | CCTGgttttagagctagaaatagcaa | |
| | aaataaggctagtccgttatcaacttgaa | | gttaaaataaggctagtccgttatcaact | |
| | aaagtggcaccGAGTCGGTGC | | tgaaaaagtggcaccGAGTCGG | |
| | CAGCCCTCGCCTCCTGGC | | TGC | |
| | TTGTCGACGACGGCGGT | | | |
| | CTCCGTCGTCAGGATCAT | | | |
| 80 | gCTTTAGAAGTTAAGGAG | 319 | gACACCGAGACCTCCAG | 320 |
| | GCGgttttagagctagaaatagcaagtt | | CCTGgttttagagctagaaatagcaa | |
| | aaaataaggctagtccgttatcaacttga | | gttaaaataaggctagtccgttatcaact | |
| | aaaagtggcaccGAGTCGGTGC | | tgaaaaagtggcaccGAGTCGG | |
| | GACCCCAGCCCTCGCGG | | TGC | |
| | CTTGTCGACGACGGCGG | | | |
| | TCTCCGTCGTCAGGATCA | | | |
| | T | | | |
| 81 | gTTTAGAAGTTAAGGAGG | 321 | gACACCGAGACCTCCAG | 322 |
| | CGAgttttagagctagaaatagcaagtt | | CCTGgttttagagctagaaatagcaa | |
| | aaaataaggctagtccgttatcaacttga | | gttaaaataaggctagtccgttatcaact | |
| | aaaagtggcaccGAGTCGGTGC | | tgaaaaagtggcaccGAGTCGG | |
| | AGACCCCAGCCCTCGGG | | TGC | |
| | CTTGTCGACGACGGCGG | | | |
| | TCTCCGTCGTCAGGATCA | | | |
| | T | | | |
| 82 | GAAGTTAAGGAGGCGAG | 323 | gACACCGAGACCTCCAG | 324 |
| | GGCgttttagagctagaaatagcaagtt | | CCTGgttttagagctagaaatagcaa | |
| | aaaataaggctagtccgttatcaacttga | | gttaaaataaggctagtccgttatcaact | |
| | aaaagtggcaccGAGTCGGTGC | | tgaaaaagtggcaccGAGTCGG | |
| | TGACAGACCCCAGCCGG | | TGC | |
| | CTTGTCGACGACGGCGG | | | |
| | TCTCCGTCGTCAGGATCA | | | |
| | T | | | |
| 83 | gAAGTTAAGGAGGCGAG | 325 | gACACCGAGACCTCCAG | 326 |
| | GGCTgttttagagctagaaatagcaa | | CCTGgttttagagctagaaatagcaa | |
| | gttaaaataaggctagtccgttatcaactt | | gttaaaataaggctagtccgttatcaact | |
| | gaaaaagtggcaccGAGTCGGT | | tgaaaaagtggcaccGAGTCGG | |
| | GCCTGACAGACCCCAGC | | TGC | |
| | GGCTTGTCGACGACGGC | | | |
| | GGTCTCCGTCGTCAGGAT | | | |
| | CAT | | | |
| 84 | gAGTTAAGGAGGCGAGG | 327 | gACACCGAGACCTCCAG | 328 |
| | GCTGgttttagagctagaaatagcaa | | CCTGgttttagagctagaaatagcaa | |
| | gttaaaataaggctagtccgttatcaactt | | gttaaaataaggctagtccgttatcaact | |
| | gaaaaagtggcaccGAGTCGGT | | tgaaaaagtggcaccGAGTCGG | |
| | GCACTGACAGACCCCAG | | TGC | |
| | GGCTTGTCGACGACGGC | | | |
| | GGTCTCCGTCGTCAGGAT | | | |
| | CAT | | | |
| 85 | gCTTGTCGGCTTTAGAAG | 329 | gAGCTAGTCAGACATGG | 330 |
| | TTAgttttagagctagaaatagcaagtt | | TGGAgttttagagctagaaatagcaa | |
| | aaaataaggctagtccgttatcaacttga | | gttaaaataaggctagtccgttatcaact | |
| | aaaagtggcaccGAGTCGGTGC | | tgaaaaagtggcaccGAGTCGG | |
| | CCCTCGCCTCCTTAAGGC | | TGC | |
| | TTGTCGACGACGGCGGT | | | |
| | CTCCGTCGTCAGGATCAT | | | |
| 86 | GTCGGCTTTAGAAGTTAA | 331 | gAGCTAGTCAGACATGG | 332 |
| | GGgttttagagctagaaatagcaagtta | | TGGAgttttagagctagaaatagcaa | |
| | aaataaggctagtccgttatcaacttgaa | | gttaaaataaggctagtccgttatcaact | |
| | aaagtggcaccGAGTCGGTGC | | tgaaaaagtggcaccGAGTCGG | |
| | CAGCCCTCGCCTCCTGGC | | TGC | |
| | TTGTCGACGACGGCGGT | | | |
| | CTCCGTCGTCAGGATCAT | | | |
| 87 | gCTTTAGAAGTTAAGGAG | 333 | gAGCTAGTCAGACATGG | 334 |
| | GCGgttttagagctagaaatagcaagtt | | TGGAgttttagagctagaaatagcaa | |
| | aaaataaggctagtccgttatcaacttga | | gttaaaataaggctagtccgttatcaact | |
| | aaaagtggcaccGAGTCGGTGC | | tgaaaaagtggcaccGAGTCGG | |
| | GACCCCAGCCCTCGCGG | | TGC | |
| | CTTGTCGACGACGGCGG | | | |
| | TCTCCGTCGTCAGGATCA | | | |
| | T | | | |
| 88 | gTTTAGAAGTTAAGGAGG | 335 | gAGCTAGTCAGACATGG | 336 |
| | CGAgttttagagctagaaatagcaagtt | | TGGAgttttagagctagaaatagcaa | |
| | aaaataaggctagtccgttatcaacttga | | gttaaaataaggctagtccgttatcaact | |
| | aaaagtggcaccGAGTCGGTGC | | tgaaaaagtggcaccGAGTCGG | |
| | AGACCCCAGCCCTCGGG | | TGC | |
| | CTTGTCGACGACGGCGG | | | |
| | TCTCCGTCGTCAGGATCA | | | |
| | T | | | |
| 89 | GAAGTTAAGGAGGCGAG | 337 | gAGCTAGTCAGACATGG | 338 |
| | GGCgttttagagctagaaatagcaagtt | | TGGAgttttagagctagaaatagcaa | |
| | aaaataaggctagtccgttatcaacttga | | gttaaaataaggctagtccgttatcaact | |
| | aaaagtggcaccGAGTCGGTGC | | | |
| | TGACAGACCCCAGCCGG | | tgaaaaagtggcaccGAGTCGG | |
| | CTTGTCGACGACGGCGG | | TGC | |
| | TCTCCGTCGTCAGGATCA | | | |
| | T | | | |
| 90 | gAAGTTAAGGAGGCGAG | 339 | gAGCTAGTCAGACATGG | 340 |
| | GGCTgttttagagctagaaatagcaa | | TGGAgttttagagctagaaatagcaa | |
| | gttaaaataaggctagtccgttatcaactt | | gttaaaataaggctagtccgttatcaact | |
| | gaaaaagtggcaccGAGTCGGT | | tgaaaaagtggcaccGAGTCGG | |
| | GCCTGACAGACCCCAGC | | TGC | |
| | GGCTTGTCGACGACGGC | | | |
| | GGTCTCCGTCGTCAGGAT | | | |
| | CAT | | | |
| 100 | gAGTTAAGGAGGCGAGG | 341 | gAGCTAGTCAGACATGG | 342 |
| | GCTGgttttagagctagaaatagcaa | | TGGAgttttagagctagaaatagcaa | |
| | gttaaaataaggctagtccgttatcaactt | | gttaaaataaggctagtccgttatcaact | |
| | gaaaaagtggcaccGAGTCGGT | | tgaaaaagtggcaccGAGTCGG | |
| | GCACTGACAGACCCCAG | | TGC | |
| | GGCTTGTCGACGACGGC | | | |
| | GGTCTCCGTCGTCAGGAT | | | |
| | CAT | | | |
| 101 | gCTTGTCGGCTTTAGAAG | 343 | gAGCTAGCTAGTCAGAC | 344 |
| | TTAgttttagagctagaaatagcaagtt | | ATGGgttttagagctagaaatagcaa | |
| | aaaataaggctagtccgttatcaacttga | | gttaaaataaggctagtccgttatcaact | |
| | aaaagtggcaccGAGTCGGTGC | | tgaaaaagtggcaccGAGTCGG | |
| | CCCTCGCCTCCTTAAGGC | | TGC | |
| | TTGTCGACGACGGCGGT | | | |
| | CTCCGTCGTCAGGATCAT | | | |
| 102 | GTCGGCTTTAGAAGTTAA | 345 | gAGCTAGCTAGTCAGAC | 346 |
| | GGgttttagagctagaaatagcaagtta | | ATGGgttttagagctagaaatagcaa | |
| | aaataaggctagtccgttatcaacttgaa | | gttaaaataaggctagtccgttatcaact | |
| | aaagtggcaccGAGTCGGTGC | | tgaaaaagtggcaccGAGTCGG | |
| | CAGCCCTCGCCTCCTGGC | | TGC | |
| | TTGTCGACGACGGCGGT | | | |
| | CTCCGTCGTCAGGATCAT | | | |
| 103 | gCTTTAGAAGTTAAGGAG | 347 | gAGCTAGCTAGTCAGAC | 348 |
| | GCGgttttagagctagaaatagcaagtt | | ATGGgttttagagctagaaatagcaa | |
| | aaaataaggctagtccgttatcaacttga | | gttaaaataaggctagtccgttatcaact | |
| | aaaagtggcaccGAGTCGGTGC | | tgaaaaagtggcaccGAGTCGG | |
| | GACCCCAGCCCTCGCGG | | TGC | |
| | CTTGTCGACGACGGCGG | | | |
| | TCTCCGTCGTCAGGATCA | | | |
| | T | | | |
| 104 | gTTTAGAAGTTAAGGAGG | 349 | gAGCTAGCTAGTCAGAC | 350 |
| | CGAgttttagagctagaaatagcaagtt | | ATGGgttttagagctagaaatagcaa | |
| | aaaataaggctagtccgttatcaacttga | | gttaaaataaggctagtccgttatcaact | |
| | aaaagtggcaccGAGTCGGTGC | | tgaaaaagtggcaccGAGTCGG | |
| | AGACCCCAGCCCTCGGG | | TGC | |
| | CTTGTCGACGACGGCGG | | | |
| | TCTCCGTCGTCAGGATCA | | | |
| | T | | | |
| 105 | GAAGTTAAGGAGGCGAG | 351 | gAGCTAGCTAGTCAGAC | 352 |
| | GGCgttttagagctagaaatagcaagtt | | ATGGgttttagagctagaaatagcaa | |
| | aaaataaggctagtccgttatcaacttga | | gttaaaataaggctagtccgttatcaact | |
| | aaaagtggcaccGAGTCGGTGC | | tgaaaaagtggcaccGAGTCGG | |
| | TGACAGACCCCAGCCGG | | TGC | |
| | CTTGTCGACGACGGCGG | | | |
| | TCTCCGTCGTCAGGATCA | | | |
| | T | | | |
| 106 | gAAGTTAAGGAGGCGAG | 353 | gAGCTAGCTAGTCAGAC | 354 |
| | GGCTgttttagagctagaaatagcaa | | ATGGgttttagagctagaaatagcaa | |
| | gttaaaataaggctagtccgttatcaactt | | gttaaaataaggctagtccgttatcaact | |
| | gaaaaagtggcaccGAGTCGGT | | tgaaaaagtggcaccGAGTCGG | |
| | GCCTGACAGACCCCAGC | | TGC | |
| | GGCTTGTCGACGACGGC | | | |
| | GGTCTCCGTCGTCAGGAT | | | |
| | CAT | | | |
| 107 | gAGTTAAGGAGGCGAGG | 355 | gAGCTAGCTAGTCAGAC | 356 |
| | GCTGgttttagagctagaaatagcaa | | ATGGgttttagagctagaaatagcaa | |
| | gttaaaataaggctagtccgttatcaactt | | gttaaaataaggctagtccgttatcaact | |
| | gaaaaagtggcaccGAGTCGGT | | tgaaaaagtggcaccGAGTCGG | |
| | GCACTGACAGACCCCAG | | TGC | |
| | GGCTTGTCGACGACGGC | | | |
| | GGTCTCCGTCGTCAGGAT | | | |
| | CAT | | | |
| 108 | AGTTAAGGAGGCGAGGG | 357 | GGAAGGTCCGCAGAGA | 358 |
| | CTGgttttagagctagaaatagcaagtt | | AGCTgttttagagctagaaatagcaa | |
| | aaaataaggctagtccgttatcaacttga | | gttaaaataaggctagtccgttatcaact | |
| | aaaagtggcaccGAGTCGGTGC | | tgaaaaagtggcaccGAGTCGG | |
| | ccggatgatcctgacgacggagaccgc | | TGCGGCCGGCTTGTCGA | |
| | cgtcgtcgacaagccggccccctcgcct | | CGACGGCGGTCTCCGTC | |
| | c | | GTCAGGATCATCCGGttct | |
| | | | ctgcgg | |
| 109 | AGTTAAGGAGGCGAGGG | 359 | AGGAAGGTCCGCAGAG | 360 |
| | CTGgttttagagctagaaatagcaagtt | | AAGCgttttagagctagaaatagca | |
| | aaaataaggctagtccgttatcaacttga | | agttaaaataaggctagtccgttatcaac | |
| | aaaagtggcaccGAGTCGGTGC | | ttgaaaaagtggcaccGAGTCGG | |
| | ATGATCCTGACGACGGA | | TGCGGCTTGTCGACGAC | |
| | GACCGCCGTCGTCGACA | | GGCGGTCTCCGTCGTCA | |
| | AGCCccctcgcctc | | GGATCATtctctgcgga | |
| 110 | AGTTAAGGAGGCGAGGG | 361 | ACACCGAGACCTCCAGC | 362 |
| | CTGgttttagagctagaaatagcaagtt | | CTGgttttagagctagaaatagcaagt | |
| | aaaataaggctagtccgttatcaacttga | | taaaataaggctagtccgttatcaacttg | |
| | aaaagtggcaccGAGTCGGTGC | | aaaaagtggcaccGAGTCGGT | |
| | ATGATCCTGACGACGGA | | GCGGCTTGTCGACGACG | |
| | GACCGCCGTCGTCGACA | | GCGGTCTCCGTCGTCAG | |
| | AGCCccctcgcctc | | GATCATgctggaggtc | |

### 8.3. Example 3

### Paired Guides Compared to Original Guides in PASTE System

The integration of cargo genes with PASTE system using paired guides instead of atgRNA and nicking guides was assessed. Paired guides, encoded in sequences presented in Table 4 and 5, were designed to target either the human or mouse NOLC1 locus.

### Material and Methods—NOLC Human Locus

Cell culture. HEK293FT cells (American Type Culture Collection (ATCC)-CRL32156) were cultured in Dulbecco's Modified Eagle Medium with high glucose, sodium pyruvate, and GlutaMAX (Thermo Fisher Scientific), additionally supplemented with 10% (v/v) fetal bovine serum (FBS) and 1× penicillin-streptomycin (Thermo Fisher Scientific).

Transfection. Cells were plated at 5-15K the day prior to transfection in a 96-well plate coated with poly-D-lysine (BD Biocoat). HEK293FT were transfected with Lipofectamine 3000 (Thermo Fisher Scientific), according to manufacturer's specifications. For PASTE insertions, 18ng of each dual guide plasmid, 64 ng cargo plasmid, and 100 ng SpCas9-RT-BXB1 encoding plasmid were delivered to each well.

Genomic DNA extraction and purification. DNA was harvested from transfected cells by removal of media, resuspension in 50 μL of QuickExtract (Lucigen), and incubation at 65° C. for 15 min, 68° C. for 15 min, and 98° C. for 10 min. After thermocycling, lysates were purified via addition of 45 μL of AMPure magnetic beads (Beckman Coulter), mixing, and two 75% ethanol wash steps. After purification, genomic DNA was eluted in 25 μL water.

Genome editing quantification by digital droplet polymerase chain reaction (ddPCR). To quantify PASTE editing efficiency by digital droplet PCR, 24 μL solutions were prepared in a 96-well plate containing: 1) 12 μL 2× ddPCR Supermix for Probes (Bio-Rad); 2) primers for amplification of the integration junction at 250 nM-900 nM; 3) FAM probe for detection of the integration junction amplicon at 250 nM; 4) 1.44 μL RPP30 HEX reference mix (Bio-Rad); 5) 0.12 μL FastDigest restriction enzyme for degradation of primer off-targets (Thermo Fisher); and 6) Sample DNA at 1-10 ng/pt. 20 μL of reaction mix was transferred to a Dg8 Cartridge (Bio-Rad) and loaded into a QX2000 droplet generator (Bio-Rad). 40 μL droplets suspended in ddPCR droplet reader oil were transferred to a new 96-well plate and thermocycled according to manufacturer's specifications. Lastly, the 96-well plate was transferred to a QX200 droplet reader (Bio-Rad) and the generated data were analyzed using Quantasoft Analysis Pro to quantify DNA editing.

### Results— NOLC Human Locus

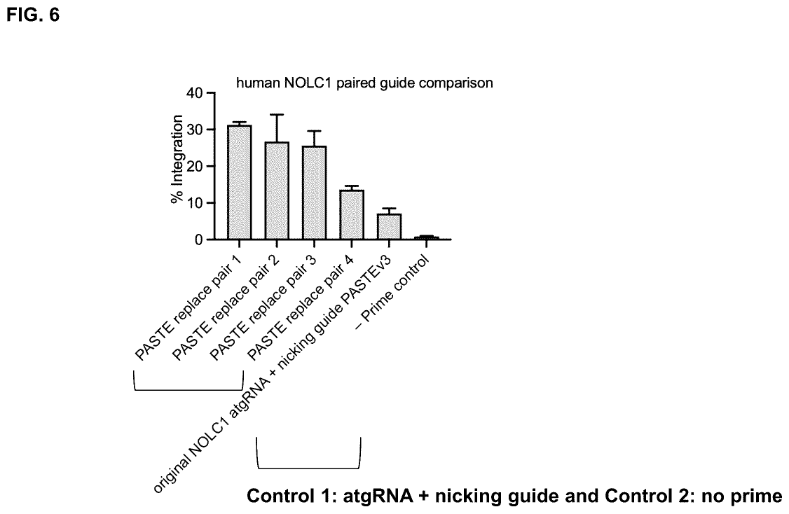

Paired guides used in conjunction with the PASTE system at the mouseNOLC1 locus demonstrated higher integration efficiency of a cargo polypeptide (i.e., eGFP) relative to a single atgRNA guide plus nicking guide (FIG. 6).

TABLE 4
Nucleic acid encoding Paired Guide Combinations for AttB insertion and subsequent
eGFP at the human NOLC1

| Pairing | Nucleic Acid Guide | SEQ | Nucleic Acid Guide | SEQ |
|---|---|---|---|---|
| Combo | Sequence 1 | ID NO | Sequence 2 | ID NO |
| 1 | GCGTATTGCCTGGAGGA | 363 | GTATTGGCCACCTCTGA | 364 |
| | TGGGTTTTAGAGCTAGA | | GAGTGTTTTAGAGCTA | |
| | AATAGCAAGTTAAAATA | | GAAATAGCAAGTTAAA | |
| | AGGCTAGTCCGTTATCA | | ATAAGGCTAGTCCGTT | |
| | ACTTGAAAAAGTGGCAC | | ATCAACTTGAAAAAGT | |
| | CGAGTCGGTGCCCGGCT | | GGCACCGAGTCGGTGC | |
| | TGTCGACGACGGCGGTC | | GGATGATCCTGACGAC | |
| | TCCGTCGTCAGGATCAT | | GGAGACCGCCGTCGTC | |
| | CCTCCTCCAGGCAAT | | GACAAGCCGGCTCAGA | |
| | | | GGTGGCC | |
| 2 | GCGTATTGCCTGGAGGA | 365 | GTATTGGCCACCTCTGA | 366 |
| | TGGGTTTTAGAGCTAGA | | GAGTGTTTTAGAGCTA | |
| | AATAGCAAGTTAAAATA | | GAAATAGCAAGTTAAA | |
| | AGGCTAGTCCGTTATCA | | ATAAGGCTAGTCCGTT | |
| | ACTTGAAAAAGTGGCAC | | ATCAACTTGAAAAAGT | |
| | CGAGTCGGTGCATGATC | | GGCACCGAGTCGGTGC | |
| | CTGACGACGGAGACCGC | | GGCTTGTCGACGACGG | |
| | CGTCGTCGACAAGCCTC | | CGGTCTCCGTCGTCAG | |
| | CTCCAGGCAAT | | GATCATCTCAGAGGTG | |
| | | | GCC | |
| 3 | GCGTATTGCCTGGAGGA | 367 | GTATTGGCCACCTCTGA | 368 |
| | TGGGTTTTAGAGCTAGA | | GAGTGTTTTAGAGCTA | |
| | AATAGCAAGTTAAAATA | | GAAATAGCAAGTTAAA | |
| | AGGCTAGTCCGTTATCA | | ATAAGGCTAGTCCGTT | |
| | ACTTGAAAAAGTGGCAC | | ATCAACTTGAAAAAGT | |
| | CGAGTCGGTGCGGCCGG | | GGCACCGAGTCGGTGC | |
| | CTTGTCGACGACGGCGG | | GGCCGGCTTGTCGACG | |
| | TCTCCGTCGTCAGGATC | | ACGGCGGTCTCCGTCG | |
| | ATCCGGTCCTCCAGG | | TCAGGATCATCCGGCT | |
| | | | CAGAGGT | |
| 4 | GCGTATTGCCTGGAGGA | 369 | GTATTGGCCACCTCTGA | 370 |
| | TGGGTTTTAGAGCTAGA | | GAGTGTTTTAGAGCTA | |
| | AATAGCAAGTTAAAATA | | GAAATAGCAAGTTAAA | |
| | AGGCTAGTCCGTTATCA | | ATAAGGCTAGTCCGTT | |
| | ACTTGAAAAAGTGGCAC | | ATCAACTTGAAAAAGT | |
| | CGAGTCGGTGCGGCTTG | | GGCACCGAGTCGGTGC | |
| | TCGACGACGGCGGTCTC | | ATGATCCTGACGACGG | |
| | CGTCGTCAGGATCATTC | | AGACCGCCGTCGTCGA | |
| | CTCCAGGCAAT | | CAAGCCCTCAGAGGTG | |
| | | | GCC | |
| 5 | GCGTATTGCCTGGAGGA | 371 | GAGCCGAGCACGAGGG | 372 |
| | TGGGTTTTAGAGCTAGA | | GATACGTTTTAGAGCT | |
| | AATAGCAAGTTAAAATA | | AGAAATAGCAAGTTAA | |
| | AGGCTAGTCCGTTATCA | | AATAAGGCTAGTCCGT | |
| | ACTTGAAAAAGTGGCAC | | TATCAACTTGAAAAAG | |
| | CGAGTCGGTGCGAACCA | | TGGCACCGAGTCGGTG | |
| | CGCGGCGAATGCCGGCG | | C | |
| | TCCGCCCCGGATGATCC | | | |
| | TGACGACGGAGACCGCC | | | |
| | GTCGTCGACAAGCCGGC | | | |
| | CTCCTCCAGGCAATACG | | | |
| | CG | | | |

Material and Methods—NOLC Mouse Locus

Cell culture. Hepal-6 cells (American Type Culture Collection (ATCC)-CRL32156) were cultured in Dulbecco's Modified Eagle Medium with high glucose, sodium pyruvate, and GlutaMAX (Thermo Fisher Scientific), additionally supplemented with 10% (v/v) fetal bovine serum (FBS) and 1× penicillin-streptomycin (Thermo Fisher Scientific).

Transfection. Cells were plated at 5-15K the day prior to transfection in a 96-well plate coated with poly-D-lysine (BD Biocoat). Hepal-6 cells were transfected with Lipofectamine 3000 (Thermo Fisher Scientific), according to manufacturer's specifications. For AttB insertion, 35.5 ng of each dual guide plasmid, and 100 ng SpCas9-RT plasmid were delivered to each well. For PASTE insertion, 19 ng of each dual guide plasmid is used, 97 ng of the PASTE plasmid (PASTEvl or PASTEv3), and 65 ng of the template plasmid was used.

Genomic DNA extraction and purification and quantitation. DNA was harvested from transfected cells by removal of media, resuspension in 50 μL of QuickExtract (Lucigen), and incubation at 65° C. for 15 min, 68° C. for 15 min, and 98° C. for 10 min. Target regions were PCR amplified with NEBNext High-Fidelity 2× PCR Master Mix (NEB) based on the manufacturer's protocol. Barcodes and adapters for Illumina sequencing were added in a subsequent PCR amplification. Amplicons were pooled and prepared for sequencing on a MiSeq (Illumina). Reads were demultiplexed and analyzed with appropriate pipelines.

Genome editing quantification by digital droplet polymerase chain reaction (ddPCR). To quantify PASTE editing efficiency by digital droplet PCR, 24 μL solutions were prepared in a 96-well plate containing: 1) 12 μL 2× ddPCR Supermix for Probes (Bio-Rad); 2) primers for amplification of the integration junction at 250 nM-900 nM; 3) FAM probe for detection of the integration junction amplicon at 250 nM; 4) 1.44 μL RPP30 HEX reference mix (Bio-Rad); 5) 0.12 μL FastDigest restriction enzyme for degradation of primer off-targets (Thermo Fisher); and 6) Sample DNA at 1-10 ng/μL. 20 μL of reaction mix was transferred to a Dg8 Cartridge (Bio-Rad) and loaded into a QX2000 droplet generator (Bio-Rad). 40 μL droplets suspended in ddPCR droplet reader oil were transferred to a new 96-well plate and thermocycled according to manufacturer's specifications. Lastly, the 96-well plate was transferred to a QX200 droplet reader (Bio-Rad) and the generated data were analyzed using Quantasoft Analysis Pro to quantify DNA editing.

### Results—NOLC Mouse Locus

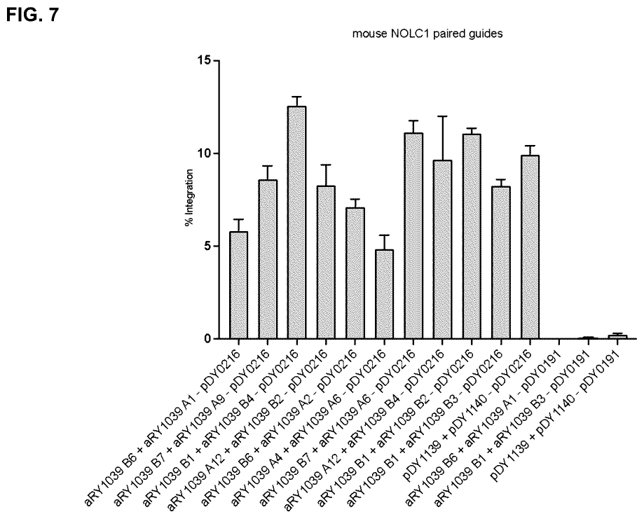

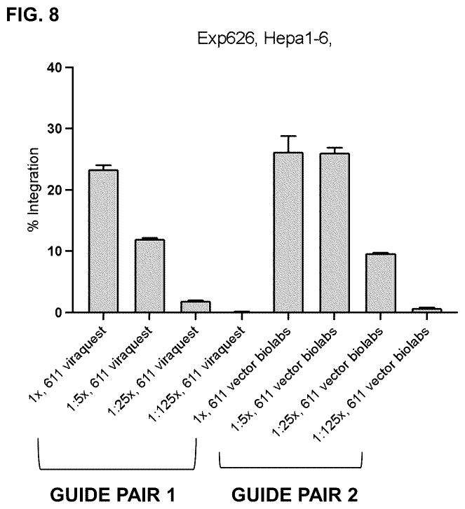

Paired guides used in conjunction with the PASTE system at the human NOLC1 locus demonstrated higher integration efficiency of a cargo polypeptide (i.e., eGFP) relative to a single atgRNA guide plus nicking guide (FIG. 7).

TABLE 5
Nucleic acid encoding Paired Guide Combinations for AttB insertion and subsequent
eGFP integration at the mouse NOLC1 locus

| Pairing | Nucleic Acid Guide | SEQ | Nucleic Acid Guide | SEQ |
|---|---|---|---|---|
| Combo | Sequence 1 | ID NO | Sequence 2 | ID NO |
| 1 | AGTTAAGGAGGCGAG | 373 | GGAAGGTCCGCAGAGAA | 374 |
| | GGCTGGTTTTAGAGC | | GCTGTTTTAGAGCTAGAA | |
| | TAGAAATAGCAAGTT | | ATAGCAAGTTAAAATAAG | |
| | AAAATAAGGCTAGTC | | GCTAGTCCGTTATCAACT | |
| | CGTTATCAACTTGAA | | TGAAAAAGTGGCACCGA | |
| | AAAGTGGCACCGAGT | | GTCGGTGCGGCCGGCTTG | |
| | CGGTGCCCGGATGAT | | TCGACGACGGCGGTCTCC | |
| | CCTGACGACGGAGAC | | GTCGTCAGGATCATCCGG | |
| | CGCCGTCGTCGACAA | | TTCTCTGCGG | |
| | GCCGGCCCCCTCGCC | | | |
| | TC | | | |
| 2 | AGTTAAGGAGGCGAG | 375 | ACACCGAGACCTCCAGCC | 376 |
| | GGCTGGTTTTAGAGC | | TGGTTTTAGAGCTAGAAA | |
| | TAGAAATAGCAAGTT | | TAGCAAGTTAAAATAAGG | |
| | AAAATAAGGCTAGTC | | CTAGTCCGTTATCAACTT | |
| | CGTTATCAACTTGAA | | GAAAAAGTGGCACCGAG | |
| | AAAGTGGCACCGAGT | | TCGGTGCGGCTTGTCGAC | |
| | CGGTGCATGATCCTG | | GACGGCGGTCTCCGTCGT | |
| | ACGACGGAGACCGCC | | CAGGATCATGCTGGAGGT | |
| | GTCGTCGACAAGCCC | | C | |
| | CCTCGCCTC | | | |
| 3 | AGTTAAGGAGGCGAG | 377 | ACACCGAGACCTCCAGCC | 378 |
| | GGCTGGTTTTAGAGC | | TGGTTTTAGAGCTAGAAA | |
| | TAGAAATAGCAAGTT | | TAGCAAGTTAAAATAAGG | |
| | AAAATAAGGCTAGTC | | CTAGTCCGTTATCAACTT | |
| | CGTTATCAACTTGAA | | GAAAAAGTGGCACCGAG | |
| | AAAGTGGCACCGAGT | | TCGGTGCATGATCCTGAC | |
| | CGGTGCGGCTTGTCG | | GACGGAGACCGCCGTCGT | |
| | ACGACGGCGGTCTCC | | CGACAAGCCGCTGGAGGT | |
| | GTCGTCAGGATCATC | | C | |
| | CCTCGCCTC | | | |
| 4 | AAGTTAAGGAGGCGA | 379 | GGAAGGTCCGCAGAGAA | 380 |
| | GGGCTGTTTTAGAGC | | GCTGTTTTAGAGCTAGAA | |
| | TAGAAATAGCAAGTT | | ATAGCAAGTTAAAATAAG | |
| | AAAATAAGGCTAGTC | | GCTAGTCCGTTATCAACT | |
| | CGTTATCAACTTGAA | | TGAAAAAGTGGCACCGA | |
| | AAAGTGGCACCGAGT | | GTCGGTGCATGATCCTGA | |
| | CGGTGCGGCTTGTCG | | CGACGGAGACCGCCGTCG | |
| | ACGACGGCGGTCTCC | | TCGACAAGCCTTCTCTGC | |
| | GTCGTCAGGATCATC | | GG | |
| | CTCGCCTCC | | | |
| 5 | AGTTAAGGAGGCGAG | 381 | AGCTAGTCAGACATGGTG | 382 |
| | GGCTGGTTTTAGAGC | | GAGTTTTAGAGCTAGAAA | |
| | TAGAAATAGCAAGTT | | TAGCAAGTTAAAATAAGG | |
| | AAAATAAGGCTAGTC | | CTAGTCCGTTATCAACTT | |
| | CGTTATCAACTTGAA | | GAAAAAGTGGCACCGAG | |
| | AAAGTGGCACCGAGT | | TCGGTGCGGCCGGCTTGT | |
| | CGGTGCCCGGATGAT | | CGACGACGGCGGTCTCCG | |
| | CCTGACGACGGAGAC | | TCGTCAGGATCATCCGGA | |
| | CGCCGTCGTCGACAA | | CCATGTCTG | |
| | GCCGGCCCCCTCGCC | | | |
| | TC | | | |
| 6 | GTCGGCTTTAGAAGT | 383 | GGAAGGTCCGCAGAGAA | 384 |
| | TAAGGGTTTTAGAGC | | GCTGTTTTAGAGCTAGAA | |
| | TAGAAATAGCAAGTT | | ATAGCAAGTTAAAATAAG | |
| | AAAATAAGGCTAGTC | | GCTAGTCCGTTATCAACT | |
| | CGTTATCAACTTGAA | | TGAAAAAGTGGCACCGA | |
| | AAAGTGGCACCGAGT | | GTCGGTGCGGCTTGTCGA | |
| | CGGTGCATGATCCTG | | CGACGGCGGTCTCCGTCG | |
| | ACGACGGAGACCGCC | | TCAGGATCATTTCTCTGC | |
| | GTCGTCGACAAGCCT | | GG | |
| | AACTTCTAA | | | |
| 7 | AGTTAAGGAGGCGAG | 385 | GGAAGGTCCGCAGAGAA | 386 |
| | GGCTGGTTTTAGAGC | | GCTGTTTTAGAGCTAGAA | |
| | TAGAAATAGCAAGTT | | ATAGCAAGTTAAAATAAG | |
| | AAAATAAGGCTAGTC | | GCTAGTCCGTTATCAACT | |
| | CGTTATCAACTTGAA | | TGAAAAAGTGGCACCGA | |
| | AAAGTGGCACCGAGT | | GTCGGTGCGGCTTGTCGA | |
| | CGGTGCATGATCCTG | | CGACGGCGGTCTCCGTCG | |
| | ACGACGGAGACCGCC | | TCAGGATCATTTCTCTGC | |
| | GTCGTCGACAAGCCC | | GG | |
| | CCTCGCCTC | | | |
| 8 | AAGTTAAGGAGGCGA | 387 | ACACCGAGACCTCCAGCC | 388 |
| | GGGCTGTTTTAGAGC | | TGGTTTTAGAGCTAGAAA | |
| | TAGAAATAGCAAGTT | | TAGCAAGTTAAAATAAGG | |
| | AAAATAAGGCTAGTC | | CTAGTCCGTTATCAACTT | |
| | CGTTATCAACTTGAA | | GAAAAAGTGGCACCGAG | |
| | AAAGTGGCACCGAGT | | TCGGTGCATGATCCTGAC | |
| | CGGTGCGGCTTGTCG | | GACGGAGACCGCCGTCGT | |
| | ACGACGGCGGTCTCC | | CGACAAGCCGCTGGAGGT | |
| | GTCGTCAGGATCATC | | C | |
| | CTCGCCTCC | | | |
| 9 | AGTTAAGGAGGCGAG | 389 | GGAAGGTCCGCAGAGAA | 390 |
| | GGCTGGTTTTAGAGC | | GCTGTTTTAGAGCTAGAA | |
| | TAGAAATAGCAAGTT | | ATAGCAAGTTAAAATAAG | |
| | AAAATAAGGCTAGTC | | GCTAGTCCGTTATCAACT | |
| | CGTTATCAACTTGAA | | TGAAAAAGTGGCACCGA | |
| | AAAGTGGCACCGAGT | | GTCGGTGCATGATCCTGA | |
| | CGGTGCGGCTTGTCG | | CGACGGAGACCGCCGTCG | |
| | ACGACGGCGGTCTCC | | TCGACAAGCCTTCTCTGC | |
| | GTCGTCAGGATCATC | | GG | |
| | CCTCGCCTC | | | |
| 10 | AGTTAAGGAGGCGAG | 391 | AGGAAGGTCCGCAGAGA | 392 |
| | GGCTGGTTTTAGAGC | | AGCGTTTTAGAGCTAGAA | |
| | TAGAAATAGCAAGTT | | ATAGCAAGTTAAAATAAG | |
| | AAAATAAGGCTAGTC | | GCTAGTCCGTTATCAACT | |
| | CGTTATCAACTTGAA | | TGAAAAAGTGGCACCGA | |
| | AAAGTGGCACCGAGT | | GTCGGTGCATGATCCTGA | |
| | CGGTGCGGCTTGTCG | | CGACGGAGACCGCCGTCG | |
| | ACGACGGCGGTCTCC | | TCGACAAGCCTCTCTGCG | |
| | GTCGTCAGGATCATC | | GA | |
| | CCTCGCCTC | | | |
| 11 | GCGTTTTACCCGGAG | 393 | GTACTGGCCACCTCCGAG | 394 |
| | CATGGGTTTTAGAGC | | AGTGTTTTAGAGCTAGAA | |
| | TAGAAATAGCAAGTT | | ATAGCAAGTTAAAATAAG | |
| | AAAATAAGGCTAGTC | | GCTAGTCCGTTATCAACT | |
| | CGTTATCAACTTGAA | | TGAAAAAGTGGCACCGA | |
| | AAAGTGGCACCGAGT | | GTCGGTGCGGCCGGCTTG | |
| | CGGTGCCCGGATGAT | | TCGACGACGGCGGTCTCC | |
| | CCTGACGACGGAGAC | | GTCGTCAGGATCATCCGG | |
| | CGCCGTCGTCGACAA | | CTCGGAGGTGGCC | |
| | GCCGGCCTGCTCCGG | | | |
| | GTAAA | | | |

### 8.4. Example 4

### Adenoviral Delivery of Paired Guides

An AdV vector cocktail to package the complete PASTE-paired guide system (i.e., Cas9-reverse transcriptase-integrase, paired guides, and genetic cargo) in viral vectors was assessed. Upon packaging and delivering the PASTE-paired guide system components across 3 AdV vectors, percent integration of eGFP at the mouse NOLC1 locus in Hepa 1-6 locus was measured by digital droplet PCR.

Material and Methods—Adenoviral delivery of PASTE and Paired Guides

Cell culture. Hepa 1-5 cellswere cultured in Dulbecco's Modified Eagle Medium with high glucose, sodium pyruvate, and GlutaMAX (Thermo Fisher Scientific), additionally supplemented with 10% (v/v) fetal bovine serum (FBS) and 1× penicillin-streptomycin (Thermo Fisher Scientific).

Transfection. Cells were plated at 5-15K the day prior to transfection in a 96-well plate coated with poly-D-lysine (BD Biocoat). HEK293FT were transfected with Lipofectamine 3000 (Thermo Fisher Scientific), according to manufacturer's specifications. For PASTE insertions, 18ng of each dual guide plasmid, 64ng cargo plasmid, and 100 ng SpCas9-RT-BXB1 encoding plasmid were delivered to each well.

Genomic DNA extraction and purification. DNA was harvested from transfected cells by removal of media, resuspension in 50 μL of QuickExtract (Lucigen), and incubation at 65° C. for 15 min, 68° C. for 15 min, and 98° C. for 10 min. After thermocycling, lysates were purified via addition of 45 μL of AMPure magnetic beads (Beckman Coulter), mixing, and two 75% ethanol wash steps. After purification, genomic DNA was eluted in 25 μL water.

Genome editing quantification by digital droplet polymerase chain reaction (ddPCR). To quantify PASTE editing efficiency by digital droplet PCR, 24 μL solutions were prepared in a 96-well plate containing: 1) 12 μL 2× ddPCR Supermix for Probes (Bio-Rad); 2) primers for amplification of the integration junction at 250 nM-900 nM; 3) FAM probe for detection of the integration junction amplicon at 250 nM; 4) 1.44 μL RPP30 HEX reference mix (Bio-Rad); 5) 0.12 μL FastDigest restriction enzyme for degradation of primer off-targets (Thermo Fisher); and 6) Sample DNA at 1-10 ng/pt. 20 μL of reaction mix was transferred to a Dg8 Cartridge (Bio-Rad) and loaded into a QX2000 droplet generator (Bio-Rad). 40 μL droplets suspended in ddPCR droplet reader oil were transferred to a new 96-well plate and thermocycled according to manufacturer's specifications. Lastly, the 96-well plate was transferred to a QX200 droplet reader (Bio-Rad) and the generated data were analyzed using Quantasoft Analysis Pro to quantify DNA editing.

AdV production and transduction. Adenoviral vectors were cloned using the AdEasy-1 system obtained from Addgene. Briefly, SpCas9-RT-P2A-Blast, Bxb1 and guide RNAs, and an EGFP cargo gene were cloned into separate adenoviral template backbones and recombined to add the full Adenoviral genome with the AdEasy-1 plasmid in BJ5183 E. coli cells. These recombined plasmids were sent to Vector BioLabs for commercial production. Additional adenoviral vectors were produced for in vivo experiments by the University of Massachusetts Medical School Viral Vector Core, as previously described (PMID: 31043560).

### Results—Adenoviral Delivery of PASTE and Paired Guides

eGFP integration into the attB site using SpCas9-RT-P2A-Blast Bxb1 and paired guides at the mouse NOLC locus in a Hepa 1-6 cell line using either a paired guide labeled, “mouse NOLC1 region forward pair with rev 38bp AttB guide 7+2” or “mouse NOLC1 region forward pair with rev 38bp AttB guide 5,” were observed.

### LIST OF SEQUENCES

TABLE 6
The amino acid sequence of exemplary DNA binding nickase.

| | | SEQ ID |
|---|---|---|
| Description | Amino Acid Sequence | NO: |
| Cas9 | MDKKYSIGLDIGTNSVGWAVITDEYKVPSKKFKVLG | 398 |
| Reference | NTDRHSIKKNLIGALLFDSGETAEATRLKRTARRRYT | |
| (Wild-Type) | RRKNRICYLQEIFSNEMAKVDDSFFHRLEESFLVEED | |
| | KKHERHPIFGNIVDEVAYHEKYPTIYHLRKKLVDST | |
| | DKADLRLIYLALAHMIKFRGHFLIEGDLNPDNSDVD | |
| | KLFIQLVQTYNQLFEENPINASGVDAKAILSARLSKS | |
| | RRLENLIAQLPGEKKNGLFGNLIALSLGLTPNFKSNF | |
| | DLAEDAKLQLSKDTYDDDLDNLLAQIGDQYADLFL | |
| | AAKNLSDAILLSDILRVNTEITKAPLSASMIKRYDEH | |
| | HQDLTLLKALVRQQLPEKYKEIFFDQSKNGYAGYID | |
| | GGASQEEFYKFIKPILEKMDGTEELLVKLNREDLLRK | |
| | QRTFDNGSIPHQIHLGELHAILRRQEDFYPFLKDNRE | |
| | KIEKILTFRIPYYVGPLARGNSRFAWMTRKSEETITP | |
| | WNFEEVVDKGASAQSFIERMTNFDKNLPNEKVLPK | |
| | HSLLYEYFTVYNELTKVKYVTEGMRKPAFLSGEQK | |
| | KAIVDLLFKTNRKVTVKQLKEDYFKKIECFDSVEISG | |
| | VEDRFNASLGTYHDLLKIIKDKDFLDNEENEDILEDI | |
| | VLTLTLFEDREMIEERLKTYAHLFDDKVMKQLKRRR | |
| | YTGWGRLSRKLINGIRDKQSGKTILDFLKSDGFANR | |
| | NFMQLIHDDSLTFKEDIQKAQVSGQGDSLHEHIANL | |
| | AGSPAIKKGILQTVKVVDELVKVMGRHKPENIVIEM | |
| | ARENQTTQKGQKNSRERMKRIEEGIKELGSQILKEHP | |
| | VENTQLQNEKLYLYYLQNGRDMYVDQELDINRLSD | |
| | YDVDHIVPQSFLKDDSIDNKVLTRSDKNRGKSDNVP | |
| | SEEVVKKMKNYWRQLLNAKLITQRKFDNLTKAERG | |
| | GLSELDKAGFIKRQLVETRQITKHVAQILDSRMNTK | |
| | YDENDKLIREVKVITLKSKLVSDFRKDFQFYKVREIN | |
| | NYHHAHDAYLNAVVGTALIKKYPKLESEFVYGDYK | |
| | VYDVRKMIAKSEQEIGKATAKYFFYSNIMNFFKTEIT | |
| | LANGEIRKRPLIETNGETGEIVWDKGRDFATVRKVL | |
| | SMPQVNIVKKTEVQTGGFSKESILPKRNSDKLIARKK | |
| | DWDPKKYGGFDSPTVAYSVLVVAKVEKGKSKKLKS | |
| | VKELLGITIMERSSFEKNPIDFLEAKGYKEVKKDLIIK | |
| | LPKYSLFELENGRKRMLASAGELQKGNELALPSKYV | |
| | NFLYLASHYEKLKGSPEDNEQKQLFVEQHKHYLDEI | |
| | IEQISEFSKRVILADANLDKVLSAYNKHRDKPIREQA | |
| | ENIIHLFTLTNLGAPAAFKYFDTTIDRKRYTSTKEVL | |
| | DATLIHQSITGLYETRIDLSQLGGD | |
| Cas9-D10A | MDKKYSIGLAIGTNSVGWAVITDEYKVPSKKFKVLG | 399 |
| | NTDRHSIKKNLIGALLFDSGETAEATRLKRTARRRYT | |
| | RRKNRICYLQEIFSNEMAKVDDSFFHRLEESFLVEED | |
| | KKHERHPIFGNIVDEVAYHEKYPTIYHLRKKLVDST | |
| | DKADLRLIYLALAHMIKFRGHFLIEGDLNPDNSDVD | |
| | KLFIQLVQTYNQLFEENPINASGVDAKAILSARLSKS | |
| | RRLENLIAQLPGEKKNGLFGNLIALSLGLTPNFKSNF | |
| | DLAEDAKLQLSKDTYDDDLDNLLAQIGDQYADLFL | |
| | AAKNLSDAILLSDILRVNTEITKAPLSASMIKRYDEH | |
| | HQDLTLLKALVRQQLPEKYKEIFFDQSKNGYAGYID | |
| | GGASQEEFYKFIKPILEKMDGTEELLVKLNREDLLRK | |
| | QRTFDNGSIPHQIHLGELHAILRRQEDFYPFLKDNRE | |
| | KIEKILTFRIPYYVGPLARGNSRFAWMTRKSEETITP | |
| | WNFEEVVDKGASAQSFIERMTNFDKNLPNEKVLPK | |
| | HSLLYEYFTVYNELTKVKYVTEGMRKPAFLSGEQK | |
| | KAIVDLLFKTNRKVTVKQLKEDYFKKIECFDSVEISG | |
| | VEDRFNASLGTYHDLLKIIKDKDFLDNEENEDILEDI | |
| | VLTLTLFEDREMIEERLKTYAHLFDDKVMKQLKRRR | |
| | YTGWGRLSRKLINGIRDKQSGKTILDFLKSDGFANR | |
| | NFMQLIHDDSLTFKEDIQKAQVSGQGDSLHEHIANL | |
| | AGSPAIKKGILQTVKVVDELVKVMGRHKPENIVIEM | |
| | ARENQTTQKGQKNSRERMKRIEEGIKELGSQILKEHP | |
| | VENTQLQNEKLYLYYLQNGRDMYVDQELDINRLSD | |
| | YDVDHIVPQSFLKDDSIDNKVLTRSDKNRGKSDNVP | |
| | SEEVVKKMKNYWRQLLNAKLITQRKFDNLTKAERG | |
| | GLSELDKAGFIKRQLVETRQITKHVAQILDSRMNTK | |
| | YDENDKLIREVKVITLKSKLVSDFRKDFQFYKVREIN | |
| | NYHHAHDAYLNAVVGTALIKKYPKLESEFVYGDYK | |
| | VYDVRKMIAKSEQEIGKATAKYFFYSNIMNFFKTEIT | |
| | LANGEIRKRPLIETNGETGEIVWDKGRDFATVRKVL | |
| | SMPQVNIVKKTEVQTGGFSKESILPKRNSDKLIARKK | |
| | DWDPKKYGGFDSPTVAYSVLVVAKVEKGKSKKLKS | |
| | VKELLGITIMERSSFEKNPIDFLEAKGYKEVKKDLIIK | |
| | LPKYSLFELENGRKRMLASAGELQKGNELALPSKYV | |
| | NFLYLASHYEKLKGSPEDNEQKQLFVEQHKHYLDEI | |
| | IEQISEFSKRVILADANLDKVLSAYNKHRDKPIREQA | |
| | ENIIHLFTLTNLGAPAAFKYFDTTIDRKRYTSTKEVL | |
| | DATLIHQSITGLYETRIDLSQLGGD | |
| Cas9- | MDKKYSIGLDIGTNSVGWAVITDEYKVPSKKFKVLG | 400 |
| H840A | NTDRHSIKKNLIGALLFDSGETAEATRLKRTARRRYT | |
| | RRKNRICYLQEIFSNEMAKVDDSFFHRLEESFLVEED | |
| | KKHERHPIFGNIVDEVAYHEKYPTIYHLRKKLVDST | |
| | DKADLRLIYLALAHMIKFRGHFLIEGDLNPDNSDVD | |
| | KLFIQLVQTYNQLFEENPINASGVDAKAILSARLSKS | |
| | RRLENLIAQLPGEKKNGLFGNLIALSLGLTPNFKSNF | |
| | DLAEDAKLQLSKDTYDDDLDNLLAQIGDQYADLFL | |
| | AAKNLSDAILLSDILRVNTEITKAPLSASMIKRYDEH | |
| | HQDLTLLKALVRQQLPEKYKEIFFDQSKNGYAGYID | |
| | GGASQEEFYKFIKPILEKMDGTEELLVKLNREDLLRK | |
| | QRTFDNGSIPHQIHLGELHAILRRQEDFYPFLKDNRE | |
| | KIEKILTFRIPYYVGPLARGNSRFAWMTRKSEETITP | |
| | WNFEEVVDKGASAQSFIERMTNFDKNLPNEKVLPK | |
| | HSLLYEYFTVYNELTKVKYVTEGMRKPAFLSGEQK | |
| | KAIVDLLFKTNRKVTVKQLKEDYFKKIECFDSVEISG | |
| | VEDRFNASLGTYHDLLKIIKDKDFLDNEENEDILEDI | |
| | VLTLTLFEDREMIEERLKTYAHLFDDKVMKQLKRRR | |
| | YTGWGRLSRKLINGIRDKQSGKTILDFLKSDGFANR | |
| | NFMQLIHDDSLTFKEDIQKAQVSGQGDSLHEHIANL | |
| | AGSPAIKKGILQTVKVVDELVKVMGRHKPENIVIEM | |
| | ARENQTTQKGQKNSRERMKRIEEGIKELGSQILKEHP | |
| | VENTQLQNEKLYLYYLQNGRDMYVDQELDINRLSD | |
| | YDVDAIVPQSFLKDDSIDNKVLTRSDKNRGKSDNVP | |
| | SEEVVKKMKNYWRQLLNAKLITQRKFDNLTKAERG | |
| | GLSELDKAGFIKRQLVETRQITKHVAQILDSRMNTK | |
| | YDENDKLIREVKVITLKSKLVSDFRKDFQFYKVREIN | |
| | NYHHAHDAYLNAVVGTALIKKYPKLESEFVYGDYK | |
| | VYDVRKMIAKSEQEIGKATAKYFFYSNIMNFFKTEIT | |
| | LANGEIRKRPLIETNGETGEIVWDKGRDFATVRKVL | |
| | SMPQVNIVKKTEVQTGGFSKESILPKRNSDKLIARKK | |
| | DWDPKKYGGFDSPTVAYSVLVVAKVEKGKSKKLKS | |
| | VKELLGITIMERSSFEKNPIDFLEAKGYKEVKKDLIIK | |
| | LPKYSLFELENGRKRMLASAGELQKGNELALPSKYV | |
| | NFLYLASHYEKLKGSPEDNEQKQLFVEQHKHYLDEI | |
| | IEQISEFSKRVILADANLDKVLSAYNKHRDKPIREQA | |
| | ENIIHLFTLTNLGAPAAFKYFDTTIDRKRYTSTKEVL | |
| | DATLIHQSITGLYETRIDLSQLGGD | |

TABLE 7
The amino acid sequence of exemplary reverse transcriptases.

| | | SEQ ID |
|---|---|---|
| Description | Amino Acid Sequence | NO: |
| M-MLV | TLNIEDEYRLHETSKEPDVSLGSTWLSDFPQAWAETG | 401 |
| Reverse | GMGLAVRQAPLIIPLKATSTPVSIKQYPMSQEARLGIK | |
| Transcript | PHIQRLLDQGILVPCQSPWNTPLLPVKKPGTNDYRPV | |
| ase | QDLREVNKRVEDIHPTVPNPYNLLSGLPPSHQWYTV | |
| Reference | LDLKDAFFCLRLHPTSQPLFAFEWRDPEMGISGQLTW | |
| (Wild- | TRLPQGFKNSPTLFDEALHRDLADFRIQHPDLILLQYV | |
| Type) | DDLLLAATSELDCQQGTRALLQTLGNLGYRASAKKA | |
| | QICQKQVKYLGYLLKEGQRWLTEARKETVMGQPTP | |
| | KTPRQLREFLGTAGFCRLWIPGFAEMAAPLYPLTKTG | |
| | TLFNWGPDQQKAYQEIKQALLTAPALGLPDLTKPFEL | |
| | FVDEKQGYAKGVLTQKLGPWRRPVAYLSKKLDPVA | |
| | AGWPPCLRMVAAIAVLTKDAGKLTMGQPLVILAPHA | |
| | VEALVKQPPDRWLSNARMTHYQALLLDTDRVQFGP | |
| | VVALNPATLLPLPEEGLQHNCLDILAEAHGTRPDLTD | |
| | QPLPDADHTWYTDGSSLLQEGQRKAGAAVTTETEVI | |
| | WAKALPAGTSAQRAELIALTQALKMAEGKKLNVYT | |
| | DSRYAFATAHIHGEIYRRRGLLTSEGKEIKNKDEILAL | |
| | LKALFLPKRLSIIHCPGHQKGHSAEARGNRMADQAA | |
| | RKAAITETPDTSTLLIENSSP | |
| M-MLV | TLNIEDEYRLHETSKEPDVSLGSTWLSDFPQAWAETG | 402 |
| Reverse | GMGLAVRQAPLIIPLKATSTPVSIKQYPMSQEARLGIK | |
| Transcript | PHIQRLLDQGILVPCQSPWNTPLLPVKKPGTNDYRPV | |
| ase | QDLREVNKRVEDIHPTVPNPYNLLSGLPPSHQWYTV | |
| Reference | LDLKDAFFCLRLHPTSQPLFAFEWRDPEMGISGQLTW | |
| (Wild-Type- | TRLPQGFKNSPTLFDEALHRDLADFRIQHPDLILLQYV | |
| C- | DDLLLAATSELDCQQGTRALLQTLGNLGYRASAKKA | |
| terminal | QICQKQVKYLGYLLKEGQRWLTEARKETVMGQPTP | |
| truncated) | KTPRQLREFLGTAGFCRLWIPGFAEMAAPLYPLTKTG | |
| | TLFNWGPDQQKAYQEIKQALLTAPALGLPDLTKPFEL | |
| | FVDEKQGYAKGVLTQKLGPWRRPVAYLSKKLDPVA | |
| | AGWPPCLRMVAAIAVLTKDAGKLTMGQPLVILAPHA | |
| | VEALVKQPPDRWLSNARMTHYQALLLDTDRVQFGP | |
| | VVALNPATLLPLPEEGLQHNCLD | |
| M-MLV | TLNIEDEYRLHETSKEPDVSLGSTWLSDFPQAWAETG | 403 |
| Reverse | GMGLAVRQAPLIIPLKATSTPVSIKQYPMSQEARLGIK | |
| Transcript | PHIQRLLDQGILVPCQSPWNTPLLPVKKPGTNDYRPV | |
| ase | QDLREVNKRVEDIHPTVPNPYNLLSGLPPSHQWYTV | |
| D200N/ | LDLKDAFFCLRLHPTSQPLFAFEWRDPEMGISGQLTW | |
| T306K/T330P/ | TRLPQGFKNSPTLFNEALHRDLADFRIQHPDLILLQYV | |
| L603W/ | DDLLLAATSELDCQQGTRALLQTLGNLGYRASAKKA | |
| W313F | QICQKQVKYLGYLLKEGQRWLTEARKETVMGQPTP | |
| | KTPRQLREFLGKAGFCRLFIPGFAEMAAPLYPLTKPG | |
| | TLFNWGPDQQKAYQEIKQALLTAPALGLPDLTKPFEL | |
| | FVDEKQGYAKGVLTQKLGPWRRPVAYLSKKLDPVA | |
| | AGWPPCLRMVAAIAVLTKDAGKLTMGQPLVILAPHA | |
| | VEALVKQPPDRWLSNARMTHYQALLLDTDRVQFGP | |
| | VVALNPATLLPLPEEGLQHNCLDILAEAHGTRPDLTD | |
| | QPLPDADHTWYTDGSSLLQEGQRKAGAAVTTETEVI | |
| | WAKALPAGTSAQRAELIALTQALKMAEGKKLNVYT | |
| | DSRYAFATAHIHGEIYRRRGWLTSEGKEIKNKDEILA | |
| | LLKALFLPKRLSIIHCPGHQKGHSAEARGNRMADQA | |
| | ARKAAITETPDTSTLLIENSSP | |
| M-MLV | TLNIEDEYRLHETSKEPDVSLGSTWLSDFPQAWAETG | 404 |
| Reverse | GMGLAVRQAPLIIPLKATSTPVSIKQYPMSQEARLGIK | |
| Transcript | PHIQRLLDQGILVPCQSPWNTPLLPVKKPGTNDYRPV | |
| ase | QDLREVNKRVEDIHPTVPNPYNLLSGLPPSHQWYTV | |
| D200N/ | LDLKDAFFCLRLHPTSQPLFAFEWRDPEMGISGQLTW | |
| T306K/T330P/ | TRLPQGFKNSPTLFNEALHRDLADFRIQHPDLILLQYV | |
| L603W/ | DDLLLAATSELDCQQGTRALLQTLGNLGYRASAKKA | |
| W313F | QICQKQVKYLGYLLKEGQRWLTEARKETVMGQPTP | |
| (Truncated | KTPRQLREFLGKAGFCRLFIPGFAEMAAPLYPLTKPG | |
| | TLFNWGPDQQKAYQEIKQALLTAPALGLPDLTKPFEL | |
| | FVDEKQGYAKGVLTQKLGPWRRPVAYLSKKLDPVA | |
| | AGWPPCLRMVAAIAVLTKDAGKLTMGQPLVILAPHA | |
| | VEALVKQPPDRWLSNARMTHYQALLLDTDRVQFGP | |
| | VVALNPATLLPLPEEGLQHNCLD | |

TABLE 8
The amino acid sequence of exemplary integrases.

| | | SEQ ID |
|---|---|---|
| Description | Amino Acid Sequence | NO: |
| Bxb1 Integrase | SRALVVIRLSRVTDATTSPERQLESCQQLCAQRG | 405 |
| | WDVVGVAEDLDVSGAVDPFDRKRRPNLARWLA | |
| | FEEQPFDVIVAYRVDRLTRSIRHLQQLVHWAEDH | |
| | KKLVVSATEAHFDTTTPFAAVVIALMGTVAQMEL | |
| | EAIKERNRSAAHFNIRAGKYRGSLPPWGYLPTRV | |
| | DGEWRLVPDPVQRERILEVYHRVVDNHEPLHLV | |
| | AHDLNRRGVLSPKDYFAQLQGREPQGREWSATA | |
| | LKRSMISEAMLGYATLNGKTVRDDDGAPLVRAE | |
| | PILTREQLEALRAELVKTSRAKPAVSTPSLLLRVLF | |
| | CAVCGEPAYKFAGGGRKHPRYRCRSMGFPKHCG | |
| | NGTVAMAEWDAFCEEQVLDLLGDAERLEKVWV | |
| | AGSDSAVELAEVNAELVDLTSLIGSPAYRAGSPQR | |
| | EALDARIAALAARQEELEGLEARPSGWEWRETGQ | |
| | RFGDWWREQDTAAKNTWLRSMNVRLTFDVRGG | |
| | LTRTIDFGDLQEYEQHLRLGSVVERLHTGMS | |

TABLE 9
The amino acid sequence of exemplary editing polypeptides.

| | | SEQ ID |
|---|---|---|
| Description | Amino Acid Sequence | NO: |
| MCP-Cas9-RT | MASNFTQFVLVDNGGTGDVTVAPSNFANGVAEW | 406 |
| | ISSNSRSQAYKVTCSVRQSSAQKRKYTIKVEVPKV | |
| | ATQTVGGVELPVAAWRSYLNMELTIPIFATNSDC | |
| | ELIVKAMQGLLKDGNPIPSAIAANSGIYSAGGGGS | |
| | GGGGSGGGGSGMKRTADGSEFESPKKKRKVDKK | |
| | YSIGLDIGTNSVGWAVITDEYKVPSKKFKVLGNT | |
| | DRHSIKKNLIGALLFDSGETAEATRLKRTARRRYT | |
| | RRKNRICYLQEIFSNEMAKVDDSFFHRLEESFLVE | |
| | EDKKHERHPIFGNIVDEVAYHEKYPTIYHLRKKLV | |
| | DSTDKADLRLIYLALAHMIKFRGHFLIEGDLNPDN | |
| | SDVDKLFIQLVQTYNQLFEENPINASGVDAKAILS | |
| | ARLSKSRRLENLIAQLPGEKKNGLFGNLIALSLGL | |
| | TPNFKSNFDLAEDAKLQLSKDTYDDDLDNLLAQI | |
| | GDQYADLFLAAKNLSDAILLSDILRVNTEITKAPL | |
| | SASMIKRYDEHHQDLTLLKALVRQQLPEKYKEIFF | |
| | DQSKNGYAGYIDGGASQEEFYKFIKPILEKMDGT | |
| | EELLVKLNREDLLRKQRTFDNGSIPHQIHLGELHA | |
| | ILRRQEDFYPFLKDNREKIEKILTFRIPYYVGPLAR | |
| | GNSRFAWMTRKSEETITPWNFEEVVDKGASAQSF | |
| | IERMTNFDKNLPNEKVLPKHSLLYEYFTVYNELT | |
| | KVKYVTEGMRKPAFLSGEQKKAIVDLLFKTNRK | |
| | VTVKQLKEDYFKKIECFDSVEISGVEDRFNASLGT | |
| | YHDLLKIIKDKDFLDNEENEDILEDIVLTLTLFEDR | |
| | EMIEERLKTYAHLFDDKVMKQLKRRRYTGWGRL | |
| | SRKLINGIRDKQSGKTILDFLKSDGFANRNFMQLI | |
| | HDDSLTFKEDIQKAQVSGQGDSLHEHIANLAGSP | |
| | AIKKGILQTVKVVDELVKVMGRHKPENIVIEMAR | |
| | ENQTTQKGQKNSRERMKRIEEGIKELGSQILKEHP | |
| | VENTQLQNEKLYLYYLQNGRDMYVDQELDINRL | |
| | SDYDVDAIVPQSFLKDDSIDNKVLTRSDKNRGKS | |
| | DNVPSEEVVKKMKNYWRQLLNAKLITQRKFDNL | |
| | TKAERGGLSELDKAGFIKRQLVETRQITKHVAQIL | |
| | DSRMNTKYDENDKLIREVKVITLKSKLVSDFRKD | |
| | FQFYKVREINNYHHAHDAYLNAVVGTALIKKYP | |
| | KLESEFVYGDYKVYDVRKMIAKSEQEIGKATAKY | |
| | FFYSNIMNFFKTEITLANGEIRKRPLIETNGETGEIV | |
| | WDKGRDFATVRKVLSMPQVNIVKKTEVQTGGFS | |
| | KESILPKRNSDKLIARKKDWDPKKYGGFDSPTVA | |
| | YSVLVVAKVEKGKSKKLKSVKELLGITIMERSSFE | |
| | KNPIDFLEAKGYKEVKKDLIIKLPKYSLFELENGR | |
| | KRMLASAGELQKGNELALPSKYVNFLYLASHYE | |
| | KLKGSPEDNEQKQLFVEQHKHYLDEIIEQISEFSK | |
| | RVILADANLDKVLSAYNKHRDKPIREQAENIIHLF | |
| | TLTNLGAPAAFKYFDTTIDRKRYTSTKEVLDATLI | |
| | HQSITGLYETRIDLSQLGGDSGGSSGGSSGSETPGT | |
| | SESATPESSGGSSGGSSTLNIEDEYRLHETSKEPDV | |
| | SLGSTWLSDFPQAWAETGGMGLAVRQAPLIIPLK | |
| | ATSTPVSIKQYPMSQEARLGIKPHIQRLLDQGILVP | |
| | CQSPWNTPLLPVKKPGTNDYRPVQDLREVNKRV | |
| | EDIHPTVPNPYNLLSGLPPSHQWYTVLDLKDAFFC | |
| | LRLHPTSQPLFAFEWRDPEMGISGQLTWTRLPQGF | |
| | KNSPTLFNEALHRDLADFRIQHPDLILLQYVDDLL | |
| | LAATSELDCQQGTRALLQTLGNLGYRASAKKAQI | |
| | CQKQVKYLGYLLKEGQRWLTEARKETVMGQPTP | |
| | KTPRQLREFLGKAGFCRLFIPGFAEMAAPLYPLTK | |
| | PGTLFNWGPDQQKAYQEIKQALLTAPALGLPDLT | |
| | KPFELFVDEKQGYAKGVLTQKLGPWRRPVAYLS | |
| | KKLDPVAAGWPPCLRMVAAIAVLTKDAGKLTMG | |
| | QPLVILAPHAVEALVKQPPDRWLSNARMTHYQA | |
| | LLLDTDRVQFGPVVALNPATLLPLPEEGLQHNCL | |
| | DILAEAHGTRPDLTDQPLPDADHTWYTDGSSLLQ | |
| | EGQRKAGAAVTTETEVIWAKALPAGTSAQRAELI | |
| | ALTQALKMAEGKKLNVYTDSRYAFATAHIHGEIY | |
| | RRRGWLTSEGKEIKNKDEILALLKALFLPKRLSIIH | |
| | CPGHQKGHSAEARGNRMADQAARKAAITETPDT | |
| | STLLIENSSPSGGSKRTADGSEFEPKKKRKV | |

TABLE 10
Nucleotide sequence of exemplary integration sites.

| | | SEQ ID |
|---|---|---|
| Description | Nucleotide Sequence | NO: |
| Lox71 | ATAACTTCGTATAATGTATGCTATACGAACGGTA | 407 |
| Lox66 | TACCGTTCGTATAATGTATGCTATACGAAGTTAT | 408 |
| attB | GGCCGGCTTGTCGACGACGGCGGTCTCCGTCGTCA | 409 |
| | GGATCATCCGG | |
| attP | CCGGATGATCCTGACGACGGAGACCGCCGTCGTC | 410 |
| | GACAAGCCGGCC | |
| attB-TT | GGCTTGTCGACGACGGCGTTCTCCGTCGTCAGGAT | 411 |
| | CAT | |
| attP-TT | GTGGTTTGTCTGGTCAACCACCGCGTTCTCAGTGG | 412 |
| | TGTACGGTACAAACCCA | |
| attB-AA | GGCTTGTCGACGACGGCGAACTCCGTCGTCAGGA | 413 |
| | TCAT | |
| attP-AA | GTGGTTTGTCTGGTCAACCACCGCGAACTCAGTGG | 414 |
| | TGTACGGTACAAACCCA | |
| attB-CC | GGCTTGTCGACGACGGCGCCCTCCGTCGTCAGGAT | 415 |
| | CAT | |
| attP-CC | GTGGTTTGTCTGGTCAACCACCGCGCCCTCAGTGG | 416 |
| | TGTACGGTACAAACCCA | |
| attB-GG | GGCTTGTCGACGACGGCGGGCTCCGTCGTCAGGA | 417 |
| | TCAT | |
| attP-GG | GTGGTTTGTCTGGTCAACCACCGCGGGCTCAGTGG | 418 |
| | TGTACGGTACAAACCCA | |
| attB-TG | GGCTTGTCGACGACGGCGTGCTCCGTCGTCAGGAT | 419 |
| | CAT | |
| attP-TG | GTGGTTTGTCTGGTCAACCACCGCGTGCTCAGTGG | 420 |
| | TGTACGGTACAAACCCA | |
| attB-GT | GGCTTGTCGACGACGGCGGTCTCCGTCGTCAGGAT | 421 |
| | CAT | |
| attP-GT | GTGGTTTGTCTGGTCAACCACCGCGGTCTCAGTGG | 395 |
| | TGTACGGTACAAACCCA | |
| attB-CT | GGCTTGTCGACGACGGCGCTCTCCGTCGTCAGGAT | 422 |
| | CAT | |
| attP-CT | GTGGTTTGTCTGGTCAACCACCGCGCTCTCAGTGG | 423 |
| | TGTACGGTACAAACCCA | |
| attB-CA | GGCTTGTCGACGACGGCGCACTCCGTCGTCAGGA | 424 |
| | TCAT | |
| attP-CA | GTGGTTTGTCTGGTCAACCACCGCGCACTCAGTGG | 425 |
| | TGTACGGTACAAACCCA | |
| attB-TC | GGCTTGTCGACGACGGCGTCCTCCGTCGTCAGGAT | 426 |
| | CAT | |
| attP-TC | GTGGTTTGTCTGGTCAACCACCGCGTCCTCAGTGG | 427 |
| | TGTACGGTACAAACCCA | |
| attB-GA | GGCTTGTCGACGACGGCGGACTCCGTCGTCAGGA | 428 |
| | TCAT | |
| attP-GA | GTGGTTTGTCTGGTCAACCACCGCGGACTCAGTGG | 429 |
| | TGTACGGTACAAACCCA | |
| attB-AG | GGCTTGTCGACGACGGCGAGCTCCGTCGTCAGGA | 430 |
| | TCAT | |
| attP-AG | GTGGTTTGTCTGGTCAACCACCGCGAGCTCAGTGG | 431 |
| | TGTACGGTACAAACCCA | |
| attB-AC | GGCTTGTCGACGACGGCGACCTCCGTCGTCAGGA | 432 |
| | TCAT | |
| attP-AC | GTGGTTTGTCTGGTCAACCACCGCGACCTCAGTGG | 433 |
| | TGTACGGTACAAACCCA | |
| attB-AT | GGCTTGTCGACGACGGCGATCTCCGTCGTCAGGAT | 434 |
| | CAT | |
| attP-AT | GTGGTTTGTCTGGTCAACCACCGCGATCTCAGTGG | 435 |
| | TGTACGGTACAAACCCA | |
| attB-GC | GGCTTGTCGACGACGGCGGCCTCCGTCGTCAGGA | 436 |
| | TCAT | |
| attP-GC | GTGGTTTGTCTGGTCAACCACCGCGGCCTCAGTGG | 437 |
| | TGTACGGTACAAACCCA | |
| attB-CG | GGCTTGTCGACGACGGCGCGCTCCGTCGTCAGGA | 438 |
| | TCAT | |
| attP-CG | GTGGTTTGTCTGGTCAACCACCGCGCGCTCAGTGG | 439 |
| | TGTACGGTACAAACCCA | |
| attB-TA | GGCTTGTCGACGACGGCGTACTCCGTCGTCAGGAT | 440 |
| | CAT | |
| attP-TA | GTGGTTTGTCTGGTCAACCACCGCGTACTCAGTGG | 441 |
| | TGTACGGTACAAACCCA | |
| C31-attB | TGCGGGTGCCAGGGCGTGCCCTTGGGCTCCCCGG | 442 |
| | GCGCGTACTCC | |
| C31-attP | GTGCCCCAACTGGGGTAACCTTTGAGTTCTCTCAG | 443 |
| | TTGGGGG | |
| R4-attB | GCGCCCAAGTTGCCCATGACCATGCCGAAGCAGT | 444 |
| | GGTAGAAGGGCACCGGCAGACAC | |
| R4-attP | AGGCATGTTCCCCAAAGCGATACCACTTGAAGCA | 445 |
| | GTGGTACTGCTTGTGGGTACACTCTGCGGGTGATG | |
| | A | |
| BT1-attB | GTCCTTGACCAGGTTTTTGACGAAAGTGATCCAGA | 446 |
| | TGATCCAGCTCCACACCCCGAACGC | |
| BT1-attP | GGTGCTGGGTTGTTGTCTCTGGACAGTGATCCATG | 447 |
| | GGAAACTACTCAGCACCACCAATGTTCC | |
| Bxb-attB | TCGGCCGGCTTGTCGACGACGGCGGTCTCCGTCGT | 448 |
| | CAGGATCATCCGGGC | |
| Bxb-attP | GTCGTGGTTTGTCTGGTCAACCACCGCGGTCTCAG | 449 |
| | TGGTGTACGGTACAAACCCCGAC | |
| TG1-attB | GATCAGCTCCGCGGGCAAGACCTTCTCCTTCACGG | 450 |
| | GGTGGAAGGTC | |
| TG1-attP | TCAACCCCGTTCCAGCCCAACAGTGTTAGTCTTTG | 451 |
| | CTCTTACCCAGTTGGGCGGGATAGCCTGCCCG | |
| C1-attB | AACGATTTTCAAAGGATCACTGAATCAAAAGTAT | 452 |
| | TGCTCATCCACGCGAAATTTTTC | |
| C1-attP | AATATTTTAGGTATATGATTTTGTTTATTAGTGTA | 453 |
| | AATAACACTATGTACCTAAAAT | |
| C370-attB | TGTAAAGGAGACTGATAATGGCATGTACAACTAT | 454 |
| | ACTCGTCGGTAAAAAGGCA | |
| C370-attP | TAAAAAAATACAGCGTTTTTCATGTACAACTATAC | 455 |
| | TAGTTGTAGTGCCTAAA | |
| K38-attB | GAGCGCCGGATCAGGGAGTGGACGGCCTGGGAGC | 456 |
| | GCTACACGCTGTGGCTGCGGTC | |
| K38-attP | CCCTAATACGCAAGTCGATAACTCTCCTGGGAGC | 457 |
| | GTTGACAACTTGCGCACCCTGA | |
| RB-attB | TCTCGTGGTGGTGGAAGGTGTTGGTGCGGGGTTG | 458 |
| | GCCGTGGTCGAGGTGGGGTGGTGGTAGCCATTCG | |
| RV-attP | GCACAGGTGTAGTGTATCTCACAGGTCCACGGTTG | 459 |
| | GCCGTGGACTGCTGAAGAACATTCCACGCCAGGA | |
| SPBC-attB | AGTGCAGCATGTCATTAATATCAGTACAGATAAA | 460 |
| | GCTGTATCTCCTGTGAACACAATGGGTGCCA | |
| SPBC-attP | AAAGTAGTAAGTATCTTAAAAAACAGATAAAGCT | 461 |
| | GTATATTAAGATACTTACTAC | |
| TP901-attB | TGATAATTGCCAACACAATTAACATCTCAATCAAG | 462 |
| | GTAAATGCTTTTTCGTTTT | |
| TP901-attP | AATTGCGAGTTTTTATTTCGTTTATTTCAATTAAGG | 463 |
| | TAACTAAAAAACTCCTTT | |
| WB-attB | AAGGTAGCGTCAACGATAGGTGTAACTGTCGTGT | 464 |
| | TTGTAACGGTACTTCCAACAGCTGGCGTTTCAGT | |
| WB-attP | TAGTTTTAAAGTTGGTTATTAGTTACTGTGATATTT | 465 |
| | ATCACGGTACCCAATAACCAATGAATATTTGA | |
| A118-attB | TGTAACTTTTTCGGATCAAGCTATGAAGGACGCAA | 466 |
| | AGAGGGAACTAAACACTTAATT | |
| A118-attP | TTGTTTAGTTCCTCGTTTTCTCTCGTTGGAAGAAG | 467 |
| | AAGAAACGAGAAACTAAAATTA | |
| BL3-attB | CAACCTGTTGACATGTTTCCACAGACAACTCACGT | 468 |
| | GGAGGTAGTCACGGCTTTTACGTTAGTT | |
| BL3-attP | GAGAATACTGTTGAACAATGAAAAACTAGGCATG | 469 |
| | TAGAAGTTGTTTGTGCACTAACTTTAA | |
| MR11-attB | ACAGGTCAACACATCGCAGTTATCGAACAATCTTC | 470 |
| | GAAAATGTATGGAGGCACTTGTATCAATATAGGA | |
| | TGTATACCTTCGAAGACACTTGTACATGATGGATT | |
| | AGAAGGCAAATCCTTT | |
| MR11-attP | CAAAATAAAAAACATTGATTTTTATTAACTTCTTT | 471 |
| | TGTGCGGAACTACGAACAGTTCATTAATACGAAG | |
| | TGTACAAACTTCCATACAAAAATAACCACGACAA | |
| | TTAAGACGTGGTTTCTA | |
| attL | ATTATTTCTCACCCTGA | 472 |
| attR | ATCATCTCCCACCCGGA | 473 |
| Vox | AATAGGTCTGAGAACGCCCATTCTCAGACGTATT | 474 |
| FRT | GAAGTTCCTATACTTTCTAGAGAATAGGAACTTC | 475 |
| Bxb1_attB_ | GGCCGGCTTGTCGACGACGGCGAACTCCGTCGTC | 476 |
| 46_AA_site | AGGATCATCCGG | |
| Bxb1_attB_ | GGCCGGCTTGTCGACGACGGCGGACTCCGTCGTC | 477 |
| 46_GA_site | AGGATCATCCGG | |
| Bxb1_attB_ | GGCCGGCTTGTCGACGACGGCGCACTCCGTCGTC | 478 |
| 46_CA_site | AGGATCATCCGG | |
| Bxb1_attB_ | GGCCGGCTTGTCGACGACGGCGTACTCCGTCGTCA | 479 |
| 46_TA_site | GGATCATCCGG | |
| Bxb1_attB_ | GGCCGGCTTGTCGACGACGGCGAGCTCCGTCGTC | 480 |
| 46_AG_site | AGGATCATCCGG | |
| Bxb1_attB_ | GGCCGGCTTGTCGACGACGGCGGGCTCCGTCGTC | 481 |
| 46_GG_site | AGGATCATCCGG | |
| Bxb1_attB_ | GGCCGGCTTGTCGACGACGGCGCGCTCCGTCGTC | 482 |
| 46_CG_site | AGGATCATCCGG | |
| Bxb1_attB_ | GGCCGGCTTGTCGACGACGGCGTGCTCCGTCGTCA | 483 |
| 46_TG_site | GGATCATCCGG | |
| Bxb1_attB_ | GGCCGGCTTGTCGACGACGGCGACCTCCGTCGTC | 484 |
| 46_AC_site | AGGATCATCCGG | |
| Bxb1_attB_ | GGCCGGCTTGTCGACGACGGCGGCCTCCGTCGTC | 485 |
| 46_GC_site | AGGATCATCCGG | |
| Bxb1_attB_ | GGCCGGCTTGTCGACGACGGCGCCCTCCGTCGTCA | 486 |
| 46_CC_site | GGATCATCCGG | |
| Bxb1_attB_ | GGCCGGCTTGTCGACGACGGCGTCCTCCGTCGTCA | 487 |
| 46_TC_site | GGATCATCCGG | |
| Bxb1_attB_ | GGCCGGCTTGTCGACGACGGCGATCTCCGTCGTCA | 488 |
| 46_AT_site | GGATCATCCGG | |
| Bxb1_attB_ | GGCCGGCTTGTCGACGACGGCGCTCTCCGTCGTCA | 489 |
| 46_CT_site | GGATCATCCGG | |
| Bxb1_attB_ | GGCCGGCTTGTCGACGACGGCGTTCTCCGTCGTCA | 490 |
| 46_TT_site | GGATCATCCGG | |
| Bxb1_attB_ | GGCTTGTCGACGACGGCGGTCTCCGTCGTCAGGAT | 421 |
| 38_GT_site | CAT | |
| Bxb1_attB_ | GGCTTGTCGACGACGGCGAACTCCGTCGTCAGGA | 413 |
| 38_AA_site | TCAT | |
| Bxb1_attB_ | GGCTTGTCGACGACGGCGGACTCCGTCGTCAGGA | 428 |
| 38_GA_site | TCAT | |
| Bxb1_attB_ | GGCTTGTCGACGACGGCGCACTCCGTCGTCAGGA | 424 |
| 38_CA_site | TCAT | |
| Bxb1_attB_ | GGCTTGTCGACGACGGCGTACTCCGTCGTCAGGAT | 440 |
| 38_TA_site | CAT | |
| Bxb1_attB_ | GGCTTGTCGACGACGGCGAGCTCCGTCGTCAGGA | 430 |
| 38_AG_site | TCAT | |
| Bxb1_attB_ | GGCTTGTCGACGACGGCGGGCTCCGTCGTCAGGA | 417 |
| 38_GG_site | TCAT | |
| Bxb1_attB_ | GGCTTGTCGACGACGGCGCGCTCCGTCGTCAGGA | 438 |
| 38_CG_site | TCAT | |
| Bxb1_attB_ | GGCTTGTCGACGACGGCGTGCTCCGTCGTCAGGAT | 419 |
| 38_TG_site | CAT | |
| Bxb1_attB_ | GGCTTGTCGACGACGGCGACCTCCGTCGTCAGGA | 432 |
| 38_AC_site | TCAT | |
| Bxb1_attB_ | GGCTTGTCGACGACGGCGGCCTCCGTCGTCAGGA | 436 |
| 38_GC_site | TCAT | |
| Bxb1_attB_ | GGCTTGTCGACGACGGCGCCCTCCGTCGTCAGGAT | 415 |
| 38_CC_site | CAT | |
| Bxb1_attB_ | GGCTTGTCGACGACGGCGTCCTCCGTCGTCAGGAT | 426 |
| 38_TC_site | CAT | |
| Bxb1_attB_ | GGCTTGTCGACGACGGCGATCTCCGTCGTCAGGAT | 434 |
| 38_AT_site | CAT | |
| Bxb1_attB_ | GGCTTGTCGACGACGGCGCTCTCCGTCGTCAGGAT | 422 |
| 38_CT_site | CAT | |
| Bxb1_attB_ | GGCTTGTCGACGACGGCGTTCTCCGTCGTCAGGAT | 411 |
| 38_TT_site | CAT | |
| Cre Lox 66 | TACCGTTCGTATAATGTATGCTATACGAAGTTAT | 408 |
| site | | |
| Cre Lox 71 | ATAACTTCGTATAATGTATGCTATACGAACGGTA | 407 |
| site | | |
| TP901-1 | TTTACCTTGATTGAGATGTTAATTGTG | 491 |
| minimal | | |
| attB site | | |
| TP901-1 | GCGAGTTTTTATTTCGTTTATTTCAATTAAGGTAA | 492 |
| minimal | CTAAAAAACTCCTTT | |
| attP site | | |
| PhiBT1 | CTGGATCATCTGGATCACTTTCGTCAAAAACCTG | 493 |
| minimal | | |
| attB site | | |
| PhiBT1 | TTCGGGTGCTGGGTTGTTGTCTCTGGACAGTGATC | 494 |
| minimal | CATGGGAAACTACTCAGCACCA | |
| attP site | | |
| Pseudo attP | CCCCAACTGGGGTAACCTTTGAGTTCTCTCAGTTG | 495 |
| site | GGG | |

Other embodiments of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

All patents and publications cited herein are incorporated by reference herein in their entirety.

1. A composition comprising:
a DNA binding nickase or a functional fragment or variant thereof;
a reverse transcriptase (RT) or a functional fragment or variant thereof;
an integration enzyme or a functional fragment or variant thereof, wherein the integration enzyme is selected from the group consisting of an integrase, a recombinase, and a reverse transcriptase; and
a guide RNA (gRNA) pair comprising:
a first heterologous gRNA or functional fragment or variant thereof, comprising:
a first spacer sequence,
a first scaffold sequence,
a first reverse transcription template sequence that comprises at least a first portion of an at least first integration recognition sequence;
a first primer binding sequence, and
a second heterologous gRNA or functional fragment or variant thereof, comprising:
a second spacer sequence,
a second scaffold sequence,
a second reverse transcription template sequence that comprises at least a second portion of the first integration recognition sequence,
a second primer binding sequence,
wherein the first heterologous RNA and the second heterologous RNA collectively encode all of the first integration recognition sequence.

2. (canceled)

3. The composition of claim 1, wherein the first primer binding sequence, the second primer binding sequence, or both, are about 9-15 nucleotides in length.

4. (canceled)

5. The composition of claim 1, wherein the at least first integration recognition sequence is about 38-46 nucleotides in length.

6. The composition of claim 1, wherein the first reverse transcription template sequence, the second reverse transcription template sequence, or both, are about 1-34 nucleotides in length.

7. The composition of claim 1, wherein the first spacer sequence, the second spacer sequence, or both, are at least about 20 nucleotides in length.

8-9. (canceled)

10. The composition of claim 1, wherein the first scaffold sequence, the second scaffold sequence, or both, are about 60-120 nucleotides in length.

11. The composition of claim 1, wherein the first reverse transcription template sequence encodes a first extended sequence and the second reverse transcription template sequence encodes a second extended sequence.

12. The composition of claim 11, wherein the first and second extended sequences comprise at least about 5 complementary nucleotides with respect to each other, wherein annealing of the complementary nucleotides forms a duplex which results in an insertion of the at least first integration recognition sequence into a target location.

13-18. (canceled)

19. The composition of claim 13, wherein the first and second heterologous gRNAs form a double stranded nucleic acid.

20. (canceled)

21. The composition of claim 1, wherein the first and second heterologous gRNAs comprise from 5′-3′ in order of the spacer sequence, the scaffold sequence, the integration sequence, and the primer binding sequence.

22. The composition of claim 1, wherein the DNA binding nickase is a Cas9-D10A, a Cas9-H840A, a Cas12a nickase, or a Cas12b nickase, or a functional fragment or variant thereof.

23. The composition of claim 1, wherein the reverse transcriptase is derived from Moloney Murine Leukemia Virus (M-MLV) reverse transcriptase, transcription xenopolymerase (RTX), avian myeloblastosis virus reverse transcriptase (AMV-RT), or Eubacterium rectale maturase RT (MarathonRT).

24. The composition of claim 1, wherein the reverse transcriptase comprises a mutation relative to the wild-type sequence.

25-26. (canceled)

27. The composition of claim 25, wherein the M-MLV reverse transcriptase domain comprises one or more of the mutations selected from the group consisting of D200N, T306K, W313F, T330P, and L603W.

28. The composition of claim 1, wherein the first scaffold sequence, the second scaffold sequence, or both, comprises at least 80% sequence identity to any one of the nucleic acid sequences set forth in Table A,

29. The composition of claim 1, wherein the integration recognition sequence comprises at least 80% sequence identity to any one of the nucleic acid sequences set forth in Table B.

30. The composition of claim 1, wherein the integration enzyme is Dre, Vika, Bxb1, φC31, RDF, FLP, φBT1, R1, R2, R3, R4, RS, TP901-1, A118, φFC1, φC1, MR11, TG1, φ370.1, Wβ, BL3, SPBc, K38, Peaches, Veracruz, Rebeuca, Theia, Benedict, KSSJEB, PattyP, Doom, Scowl, Lockley, Switzer, Bob3, Troube, Abrogate, Anglerfish, Sarfire, SkiPole, ConceptII, Museum, Severus, Airmid, Benedict, Hinder, ICleared, Sheen, Mundrea, BxZ2, φRV, retrotransposases encoded by R2, L1, Tol2 Tel, Tc3, Mariner (Himar 1), Mariner (mos 1), or Minos, or any functional fragments or variants thereof.

31. (canceled)

32. The composition of claim 1, wherein the integration sequence is an attB sequence, an attP sequence, an attL sequence, an attR sequence, a Vox sequence, a FRT sequence, or a functional fragment or variant thereof

33-35. (canceled)

36. The composition of claim 1, wherein said DNA binding nickase is a Cas9-D10A, a Cas9-H840A, a Casl2a/b/c/d/e/f/h/i/j, or a functional fragment or variant thereof

37. A method of site-specifically integrating an exogenous nucleic acid into a cell genome, the method comprising:
(a) incorporating an integration sequence at a target location in the cell genome by introducing into a cell:
i. a DNA binding nickase or a functional fragment or variant thereof;
ii. a reverse transcriptase (RT) or a functional fragment or variant thereof; and
iii. a guide RNA (gRNA) pair comprising:
a first heterologous gRNA or functional fragments or variants thereof, comprising:
a first spacer sequence,
a first scaffold sequence,
a first reverse transcription template sequence that comprises at least a first portion of an at least first integration recognition sequence;
a first primer binding sequence
and
a second heterologous gRNA or functional fragments or variants thereof, comprising:
a second spacer sequence,
a second scaffold sequence,
a second reverse transcription template sequence that comprises at least a second portion of the first integration recognition sequence,
a second primer binding sequence
wherein:
the first and second heterologous gRNAs interact with the DNA binding nickase and target the target location in the cell genome,
the DNA binding nickase nicks a strand of the cell genome, and
the reverse transcriptase reverse transcribes (i) the first reverse transcription template sequence into a first extended sequence that encodes the at least first portion of the first integration recognition sequence and (ii) the second reverse transcription template sequence into a second extended sequence that encodes the at least second portion of the first integration recognition sequence,
the first and second extended sequences comprise at least about 5 complementary nucleotides with respect to each other, wherein annealing of the complementary nucleotides forms a duplex which results in the insertion of the at least first integration recognition sequence into the target location; and
(b) integrating the nucleic acid into the cell genome by introducing into the cell:
i. a DNA or RNA strand comprising the nucleic acid linked to a sequence that is complementary or associated to the integration sequence; and
ii. an integration enzyme or a functional fragment or variant thereof, wherein the integration enzyme is selected from the group consisting of an integrase, a recombinase, and a reverse transcriptase,
wherein the integration enzyme incorporates the nucleic acid into the cell genome at the at least first integration recognition sequence by integration, recombination, or reverse transcription of the sequence that is complementary or associated to the integration sequence, thereby introducing the nucleic acid into the target location of the cell genome of the cell.

38-77. (canceled)

78. A gRNA pair that specifically binds to a DNA binding nickase, wherein the gRNA pair comprises a first heterologous gRNA or functional fragments or variants thereof, and a second heterologous gRNA or functional fragments or variants thereof, and wherein the first and second heterologous gRNAs separately comprise a scaffold sequence, a primer binding sequence, an integration sequence, a spacer sequence, and optionally a reverse transcription template sequence.

79. A polypeptide comprising a DNA binding nickase linked to a reverse transcriptase, an integration enzyme, and a gRNA pair.

80. (canceled)
